(19) United States
(12) Patent Application Publication
(10) Pub. No.: US 2026/0151168 A1
(43) Pub. Date: Jun. 4, 2026

(54) METHOD OF REPAIRING BONE FRACTURE IN PATIENT

(71) Applicant: Imam Abdulrahman Bin Faisal University, Dammam (SA)

(72) Inventors: Mohammed Amin BARAJAA, Dammam (SA); Noor Wail Mohamed TAFISH, Dammam (SA); Rahaf Tarig Mohamd ABDALHALEEM, Dammam (SA); Khadijah Raed A. ALBASHA, Dammam (SA); Mashael Wael F. ALHARBI, Dammam (SA); Anwar Taqi A. ALGHAFLI, Dammam (SA); Wala’a ALMURAIKHI, Dammam (SA)

(73) Assignee: Imam Abdulrahman Bin Faisal University, Dammam (SA)

(21) Appl. No.: 19/232,834

(22) Filed: Jun. 9, 2025

Related U.S. Application Data
(60) Provisional application No. 63/726,953, filed on Dec. 2, 2024.

(51) Int. Cl. A61B 17/80; A61B 17/86

(52) U.S. Cl. CPC A61B 17/8052; A61B 17/866

(57) ABSTRACT

A method of repairing a bone fracture in a patient specifically configured for repairing critical-sized segmental bone fractures by utilizing patient-specific three-dimensional printed implants. The method includes imaging the bone fracture in need of repair; generating a fracture model of the bone fracture based on the imaging; forming a model of a bone repair implant based on the fracture model; manufacturing the bone repair implant using an additive manufacturing method based on the model; and surgically implanting the bone repair implant adjacent to the bone fracture in the patient. The bone repair implant includes a bone scaffold, a support plate, and a screw. The bone repair implant is constructed of a biodegradable material such as polylactic acid, polyglycolic acid, or poly lactic acid glycolic acid.

### CROSS REFERENCE TO RELATED APPLICATIONS

The present application claims priority to U.S. Patent Application No. 63/726,953, which was filed Dec. 2, 2024, and which is incorporated herein by reference in its entirety for all purposes.

### BACKGROUND

### Technical Field

The present disclosure relates generally to the field of orthopedic implants for bone repair. More specifically, the present disclosure relates to biodegradable bone repair implant systems and methods for treating critical-sized segmental bone fractures using patient-specific, three-dimensionally printed implant components.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

Segmental bone fractures are types of bone fractures, in which a single bone is broken in at least two places, resulting in three separate bone segments. These fractures often result in the emergence of empty defects in the bone. Many reasons, including car accidents, work/compact injuries, or falls from high places, can result in these fractures. Tumor removal can also result in such fractures. Segmental bone fractures can also be the resultant of pathological disorders, which take place when a bone is weakened by an underlying illness such as bone cancer or osteoporosis. In cases of minor bone injuries, known as “non-critical segmental bone defects”, the bone tissue can regenerate and heal itself via the body's natural healing mechanism. However, in cases of major bone injures, known as “critical-sized segmental bone defects”, where the bone defect exceeds 2 cm in length, the natural regeneration of bone is not. This necessitates complex and invasive surgical interventions.

Critical-sized segmental bone fractures not only pose a significant negative effect on the patients' overall well-being, but also place a significant financial burden on the healthcare ecosystem. Due to the complexity of such fractures, treating them is often expensive, necessitating multiple surgical procedures and special tools such as bone grafts, plates, screws, and fixation systems [J. R. Perez, et. al., Front. Bioeng. Biotechnol., vol. 6, p. 105, July 2018]. Over 3 million cases of bone fracture injuries are reported each year in the United States, inflicting a significant burden on the healthcare system. The annual cost of treating bone fracture injuries of people is estimated to exceed $25 billion in the United States. Likewise, bone fracture injuries are a prevailing concern in Saudi Arabia, with over 174,00 cases documented annually. The associated economic costs of treating these injuries exceed 2.38 billion Riyals annually, enclosing medical expenses, rehabilitation, and lost productivity. In addition, individuals with critical-sized segmental bone fractures often need lengthy hospitalization and post-operative rehabilitation programs, which can all contribute to the overall treatment cost. In the United States alone, the annual cost of treating such fractures is estimated to be $5 billion. This implies that a paradigm shift is required to find novel and innovative alternative solutions that not only are cost-effective, but can also effectively treat such fractures.

Critical-sized segmental bone fractures pose significant challenges in clinical practice, often requiring complex surgical interventions and prolonged healing times. Defect size, fracture properties, and local biology are factors that contribute to determining treatment options. Autografts, allografts, and metallic implants could successfully be employed to treat bone defects. However, these techniques have limitations that motivate the exploration of alternative approaches [B. J. Schultz, et. al., Bull. Hosp. Jt. Dis. 2013, vol. 80, no. 1, pp. 53-64, March 2022].

One of the current clinical treatment options for treating critical-sized segmental bone fractures is the utility of biological bone grafts. In this treatment option, the graft is transplanted to fill the defect area, which should result in a fully integrated regions of new bone. Autografts, bone tissues harvested from the patient's own body, is one example of biological bone grafts. During the surgery, surgeons commonly obtain bone graft from a patient's pelvic bone with a threshold of (<5 cm). The obtained autograft is then reshaped and implanted with the support of screws or pins into the defect area. The advantage of this technique is that it does not impose the risk of immune rejection since the graft is taken from the patient's own body. Therefore, the success rate of autograft surgeries is often high. Other advantages include low immunogenicity, abundance of autologous progenitor cells, and good integration with the host bone. However, autografts exhibit serious limitations that should be considered. One of the common limitations is the possibility of injuring the muscles, nerves, or blood vessels near the harvested bone area, ultimately resulting in chronic pain and/or disability. Other limitations include the limited availability of autografts and the prolonged hospitalization time due to the donor-site morbidity caused by the harvesting of an autologous tissue. Geometry constrains is another common limitation of autografts. Since autografts are often obtained from the pelvic bone, they need to be reshaped to match the defect's geometry. Yet, it is not possible to precisely control the shape of the harvested bone to fit the shape and dimensions of the defect site. This can ultimately result in suboptimal bone regeneration and/or irregularity in the shape of the forming bone.

Another popular treatment option for treating critical-sized segmental bone fractures is through the utility of allografts, which is a piece of bone obtained from a different individual (often obtained from cadavers). Unlike autograft procedures, allograft procedures are less invasive since the bone graft is obtained from a different individual. In addition, allografts can readily be supplied since they can be obtained from any cadaver. However, allografts exhibit a myriad of limitations including the risks of disease transmission, immune rejections, and limited graft integration. Another limitation of allografts pertains to geometric constraints. While allografts sourced from anatomically similar sites can be procured, ensuring an exact match in dimensions with the defect site is not always possible.

Unlike the above discussed bone grafts which are classified as biological tissues, metallic implants are medical implants manufactured by humans. Metallic implants are used to sustain or replace missing or damaged vital structures, or to enhance their mechanical functionality. Although metallic implants provide robust structural and mechanical stability, they often lack the biological properties required for optimal bone regeneration and may cause long-term complications such as stress shielding and implant-related infections and are nondegradable.

Additionally, metallic biomaterials may cause corrosion reactions in body fluids. These interactions often result in the release of metallic ions, which are hazardous when released in enormous quantities [A. Dehghanghadikolaei and B. Fotovvati, Materials, vol. 12, no. 11, p. 1795 June 2019].

Bone defects occur when there is a disturbance in the bone remodeling process and a dysfunction in the bone matrix induced by trauma or surgery. Generally, critical-sized bone defects, a defect longer than 2 cm, do not undergo a self-healing cycle and need surgical intervention. According to Haines et. al., the defect size and the infection extent are the main factors influencing treatment success [N. M. Haines, et. al., J. Orthop. Trauma, vol. 30, no. 5, pp. e158-163, May 2016]. Endochondral ossification (EO) and intra-membranous ossification (IMO) mechanisms play significant roles in bone self-healing after an injury. The EO mechanism creates fibrous tissue, granulation tissue, and cartilage tissue by stimulating an inflammation reaction between the hematoma, which is found at the fractured end and the bone marrow cavity, and the surroundings. In return, osteoblasts attack and replace chondrocytes, ultimately forming bone tissue. On the other hand, IMO causes the periosteum to be thickened and calcified by raising the number of osteoblast cells in the inner and outer periosteum. Then, the fracture ends are connected. Critical-sized bone defects are primarily repaired through EO and IMO mechanisms, facilitated by the placement of a tissue-engineered scaffold at the defect site. This scaffold, typically a 3D structure or framework fabricated from biocompatible materials like polymers, ceramics, or natural biomaterials, is specifically engineered to fill tissue defects and provide structural support while guiding the growth of cells and tissues.

With the rapid development of modern technology, some innovations have emerged in medicine, engineering, and industry. These innovations have contributed to the treatment of many incurable diseases, including bone defects. Several techniques have been developed to fabricate 3D tissue-engineered scaffolds for bone tissue regeneration. A tissue engineering scaffold is a 3D structure or framework made from biocompatible materials (such as polymers, ceramics, or natural biomaterials) that is designed to fill tissue defects and support and guide the growth of cells and tissues. The porosity, pore structure, and interconnectivity have considerable influence on the mechanical and biological properties of bone scaffolds. Thus, the chosen fabrication technique is critical and should ensure the fabrication of precise porous structures and reproducible scaffolds. The proceeding paragraphs discuss the various fabrication methods developed to produce 3D scaffolds for bone tissue regeneration. These methods include the sintered microsphere technique, salt leaching technique, molding technique, and electrospinning technique.

Microsphere-based scaffolds have drawn the attention of many researchers because of their simplicity, controlled morphology, and mechanical characteristics which resemble that of the native bone tissue. The first microsphere-based sintered scaffolds, which comprised PLGA, were developed in 1996 by Cato Laurencin's laboratory. These scaffolds showed excellent porosity and mechanical properties, making them suitable for bone tissue engineering. Borden et al. pioneered the production of microsphere-based sintered scaffolds in 2001, which combine the advantages of porous scaffolds for loading and transporting cells with the controlled release capabilities of microspheres. This technique offers the additional benefit of creating shape-specific structures with precisely interconnected pores and spatiotemporal control. Different approaches have been used for fabricating microsphere-based sintered scaffolds, including heat sintering, solvent/nonsolvent sintering methods (acetone and ethanol treatment), and solvent vapor treatment (dichloromethane). Despite its advantages, the microsphere-based sintered technique requires high temperatures and the use of organic solvents, which may restrict its usefulness in biomedical applications [A. R. C. Duarte, et. al., Int. J. Pharm., vol. 332, no. 1-2, pp. 132-139, March 2007].

The salt leaching technique is a common method for producing 3D scaffolds for bone tissue engineering applications. It involves incorporating salt crystals, like sodium chloride (NaCl), into a polymer solution. Once the polymer solidifies, the salt is leached out with a solvent/water, leaving behind a porous network within the scaffold. The primary advantage of this technique lies in its efficient utilization of small amounts of polymer, minimizing waste. However, while it facilitates the fabrication of pores of varying sizes with ease, it does have limitations. For instance, scaffolds fabricated by the salt leaching technique may exhibit irregular pore structures and inadequate control over the pore interconnectivity, essential for supporting cell attachment, growth, and nutrient transport within the scaffold. Furthermore, the pore volume and interconnectivity are often hard to reproduce in scaffolds fabricated by the salt leaching method [D. Y. Kwon, et. al., Polymers, vol. 12, no. 10, pp. 2210, 2020].

The molding technique refers to the process of shaping or forming a material into a desired shape using a mold. This technique is commonly used for scaffold fabrication with specific geometries and structures in the field of tissue engineering. There are different examples of molding techniques used depending on the desired scaffold properties, material characteristics, and the intended application of the scaffold. Researchers and engineers continue to develop new and innovative molding techniques to improve scaffold fabrication processes. These molding techniques offer several advantages for scaffold fabrication, including the ability to create scaffolds of any desired shape by simply changing the mold, the ability to produce complex structures, and the potential for high reproducibility and scalability [R. M. Allaf, Functional 3D Tissue Engineering Scaffolds, Materials Technologies And Applications, pp 75-100, 2018]. The choice of molding technique depends on the specific requirements of the tissue engineering application and the desired scaffold properties. However, the molding technique still possesses limitations that need to be considered, such as inefficient material usage and limited control over scaffold internal architecture.

Electrospinning is an innovative technique that employs electrostatic forces to create fibrous scaffolds using biocompatible polymers. To carry out the electrospinning process, three essential components are required: a nozzle tip connected to a high voltage direct current (HVDC) source, a flow rate controller, and a grounded collector. When an electric field is applied between the nozzle tip and the grounded collector, it triggers the formation of a polymeric droplet at the nozzle's end. As the strength of the electric field intensifies, the droplet elongates, taking on a conical shape known as the Taylor cone. Within the Taylor cone, the electrostatic force overcomes surface tension, resulting in the generation of a liquid jet. This jet undergoes bending instabilities, leading to the generation of fibers and the formation of a randomly oriented fibrous mat [M. Rahmati, et. al., Progress in Materials Science, vol. 117, pp. 100721, April 2021]. The remarkable surface-to-volume ratio of nanofibers, combined with their ability to fabricate scaffolds that mimic extracellular matrices, facilitate crucial processes such as cell migration, proliferation, adhesion, and differentiation. These properties make electrospinning highly desirable for tissue engineering applications. However, electro-spun scaffolds do face significant practical limitations. Firstly, they exhibit poor cell penetration, indicating that cells may struggle to infiltrate the scaffold structure effectively. Also, these scaffolds are not suitable for load-bearing applications due to their low mechanical strength.

Accordingly, it is one object of the present disclosure to provide a bone repair method and system to address these limitations.

### SUMMARY

According to a first aspect, the present disclosure relates to a method of repairing a bone fracture in a patient. In some embodiments, the method comprises imaging the bone fracture in need of repair; generating a fracture model of the bone fracture based on the imaging; forming a model of a bone repair implant based on the fracture model; manufacturing the bone repair implant using an additive manufacturing method based on the model; and surgically implanting the bone repair implant adjacent to the bone fracture in the patient. In some embodiments, the bone repair implant comprises a bone scaffold, a support plate, and a screw. In some embodiments, the bone scaffold comprises a network of scaffold members defining an interconnected pore network and has a shape configured to replace a missing bone portion from the bone fracture. In some embodiments, support plate has an inner surface profile configured to match an outer surface profile of the bone fracture in need of repair in an area covered by the support plate and an outer surface profile which smoothly transitions to match an outer surface profile of the bone fracture in need of repair in an area not covered by the support plate such that the bone repair implant is devoid of sharp edges. In some embodiments, the bone repair implant is constructed of a biodegradable material which is at least one selected from the group consisting of polylactic acid, polyglycolic acid, and poly lactic acid glycolic acid.

In some embodiments, the bone fracture in need of repair includes a proximal bone portion, the missing bone portion, and a distal bone portion.

In some embodiments, the support plate is configured to secure the bone scaffold, a proximal bone portion, and a distal bone portion in a repair configuration.

In some embodiments, the support plate is configured to be attached to at least one selected from the group consisting of the proximal bone portion and the distal bone portion using the screw.

In some embodiments, the support plate is configured to be attached to both the proximal bone portion using a first screw and the distal bone portion using a second screw.

In some embodiments, the support plate is configured to be further attached to the bone scaffold using a screw.

In some embodiments, the biodegradable material comprises polylactic acid.

In some embodiments, the bone scaffold has a pore volume of 25 to 60% of a total volume of the bone scaffold.

In some embodiments, the support plate has a thickness of 5 to 7 mm.

In some embodiments, the screw has a shank diameter of at least 8 mm.

In some embodiments, the screw has a head diameter of at least 9 mm.

In some embodiments, the method further comprises calculating a mechanical stress response of the model of a bone repair implant to an expected mechanical stress.

In some embodiments, the expected mechanical stress is based on a location of the bone fracture in need of repair.

In some embodiments, the expected mechanical stress comprises at least one selected from the group consisting of a compressive stress, a torsional stress, and a bending stress. In some embodiments, the method further comprises, when the mechanical stress response of the model of the bone repair implant to the expected mechanical stress is calculated to be unacceptable, adjusting the model of the bone repair implant to produce an acceptable mechanical stress response.

In some embodiments, the bone repair implant comprises a bone scaffold, a support plate, and a screw; and the adjusting of the model of the bone repair implant includes at least one selected from the group consisting of adjusting a pore volume of the bone scaffold, adjusting a thickness of the support plate, adjusting a length of the support plate, adjusting width profile of the support plate, adjusting a screw placement location, and adjusting a screw number of the bone repair implant.

In some embodiments, the imaging is at least one selected from the group consisting of computerized tomography, magnetic resonance imaging, and nuclear bone scanning.

In some embodiments, the bone repair implant further comprises a radiopaque marker.

In some embodiments, the additive manufacturing method is at least one selected from the group consisting of fused deposition modeling, selective laser sintering, and stereolithography.

In some embodiments, the patient does not require an additional surgery to remove the bone repair implant.

The foregoing general description of the illustrative embodiments and the following detailed description thereof are merely exemplary aspects of the teachings of this disclosure, and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 is an exemplary flowchart of a method of repairing a bone fracture in a patient, according to certain embodiments.

FIG. 2A is an exemplary cross-sectional view of structural components of cortical and cancellous bone, according to certain embodiments.

FIG. 2B is an exemplary CT scan image of a 66-year old male showing critical-sized segmental bone fractures, according to certain embodiments.

FIG. 2C is an exemplary CT scan image of a 66-year old male showing critical-sized segmental bone fractures, according to certain embodiments.

FIG. 3 is an exemplary schematic diagram of a fused deposition modeling 3D printer, according to certain embodiments.

FIG. 4 is an exemplary schematic diagram of a selective laser sintering 3D printer, according to certain embodiments.

FIG. 5 is an exemplary schematic diagram of a stereolithography 3D printer, according to certain embodiments.

FIG. 6 is an exemplary graphical illustration of a process showing implementation of a combination of different biomaterials with 3D printing technology to form bone scaffolds, according to certain embodiments.

FIG. 7A is an exemplary diagram showing a side view of a final design of patient-specific and biodegradable 3D-printed bone substitutes, bone screws, and bone plates created using CT scan images obtained from a 54-year old male patient, according to certain embodiments.

FIG. 7B is an exemplary diagram showing the front view of a final design of patient-specific and biodegradable 3D-printed bone substitutes, bone screws, and bone plates created using CT scan images obtained from a 54-year old male patient, according to certain embodiments.

FIG. 7C is an exemplary diagram showing a side view of a final design of patient-specific and biodegradable 3D-printed bone substitutes, bone screws, and bone plates created using CT scan images obtained from a 54-year old male patient, according to certain embodiments.

FIG. 8 is an exemplary diagram of a process showing 3D reconstruction and segmentation of CT scan images and generation of STL file for processing of bone fracture data, according to certain embodiments.

FIG. 9 is an exemplary diagram showing a process of developing a patient-specific 3D bone scaffold model using computer-aided design software, according to certain embodiments.

FIG. 10A is an exemplary diagram showing the orthogonal view of patient-specific 3D bone scaffold models with 50% pore volumes matched to patient anatomy, according to certain embodiments.

FIG. 10B is an exemplary diagram showing the orthogonal view of patient-specific 3D bone scaffold models with 40% pore volumes matched to patient anatomy, according to certain embodiments.

FIG. 10C is an exemplary diagram showing the orthogonal view of patient-specific 3D bone scaffold models with 30% pore volumes matched to patient anatomy, according to certain embodiments.

FIG. 10D is an exemplary diagram showing the top view of patient-specific 3D bone scaffold models with 50% pore volumes matched to patient anatomy, according to certain embodiments.

FIG. 10E is an exemplary diagram showing the top view of patient-specific 3D bone scaffold models with 40% pore volumes matched to patient anatomy, according to certain embodiments.

FIG. 10F is an exemplary diagram showing the top view of patient-specific 3D bone scaffold models with 30% pore volumes matched to patient anatomy, according to certain embodiments.

FIG. 11 is an exemplary diagram showing design steps for a patient-specific bone plate using a bone reference to create plate geometry of different thicknesses and adding screw holes, according to certain embodiments.

FIG. 12A is an exemplary diagram showing orthopedic bone screws of 6 millimeter diameters, according to certain embodiments.

FIG. 12B is an exemplary diagram showing orthopedic bone screws of 7 millimeter diameters, according to certain embodiments.

FIG. 12C is an exemplary diagram showing orthopedic bone screws of 8 millimeter diameters, according to certain embodiments.

FIG. 13 is an exemplary diagram showing patient-specific bone screws with lengths matched to bone diameter at different locations, according to certain embodiments.

FIG. 14 is an exemplary flowchart showing the design process for patient-specific bone scaffold, plates, and screws, according to certain embodiments.

FIG. 15 is an exemplary diagram showing selection of polylactic acid material properties in computer-aided design software, according to certain embodiments.

FIG. 16 is an exemplary diagram showing finite element analysis setup steps before running simulation, according to certain embodiments.

FIG. 17A is an exemplary diagram showing stress and deformation data of a bone scaffold with 50% pore volume after finite element analysis, according to certain embodiments.

FIG. 17B is an exemplary diagram showing stress and deformation data of a bone scaffold with 30% pore volume after finite element analysis, according to certain embodiments.

FIG. 17C is an exemplary diagram showing stress and deformation data of a bone scaffold with 40% pore volume after finite element analysis, according to certain embodiments.

FIG. 18A is an exemplary diagram showing stress and deformation data of a bone plate with 5 millimeter thickness after finite element analysis, according to certain embodiments.

FIG. 18B is an exemplary diagram showing stress and deformation data of a bone plate with 6 millimeter thickness after finite element analysis, according to certain embodiments.

FIG. 18C is an exemplary diagram showing stress and deformation data of a bone plate with 7 millimeter thickness after finite element analysis, according to certain embodiments.

FIG. 19A is an exemplary diagram showing stress and deformation data of a bone screw with 6 millimeter diameter after finite element analysis, according to certain embodiments.

FIG. 19B is an exemplary diagram showing stress and deformation data of a bone screw with 7 millimeter diameter after finite element analysis, according to certain embodiments.

FIG. 19C is an exemplary diagram showing stress and deformation data of a bone screw with 8 millimeter diameter after finite element analysis, according to certain embodiments.

FIG. 20A is an exemplary diagram showing a Prusa TRILAB AzteQ Industrial 3D printer configured for fabricating bone scaffolds using polylactic acid filament, according to certain embodiments.

FIG. 20B is an exemplary diagram showing a MakerBot Replicator Z18 3D printer configured for fabricating bone plates and screws using polylactic acid filament, according to certain embodiments.

FIG. 21A is an exemplary diagram showing the side view of the process of gold-sputter coating bone scaffold samples for scanning electron microscopy analysis, according to certain embodiments.

FIG. 21B is an exemplary diagram showing the top view of the process of gold-sputter coating bone scaffold samples for scanning electron microscopy analysis, according to certain embodiments.

FIG. 21C is an exemplary diagram showing a scanning electron microscope setup used for imaging the microstructure of fabricated bone scaffolds, according to certain embodiments.

FIG. 21D is an exemplary diagram showing a scanning electron microscope setup used for imaging the microstructure of fabricated bone scaffolds, according to certain embodiments.

FIG. 22A is an exemplary diagram showing compression mechanical testing setup used to assess mechanical strength of bone scaffolds, according to certain embodiments.

FIG. 22B is an exemplary diagram showing four-point bending mechanical testing setup used to evaluate mechanical strength of bone plates, according to certain embodiments.

FIG. 22C is an exemplary diagram showing torsion mechanical testing setup used to assess mechanical strength of bone screws, according to certain embodiments.

FIG. 23A is an exemplary diagram showing the side view of samples positioned in a water bath for biodegradation assessment, according to certain embodiments.

FIG. 23B is an exemplary diagram showing the top view of samples positioned in a water bath for biodegradation assessment, according to certain embodiments.

FIG. 23C is an exemplary diagram showing bone scaffolds, plates, and screws samples arranged for biodegradation testing, according to certain embodiments.

FIG. 24 is an exemplary diagram showing the process steps for biocompatibility evaluations including cell viability and growth assessments using PBS solution and growth media over a 7-day period, according to certain embodiments.

FIG. 25A is an exemplary scanning electron microscopy images showing the microstructure of 30% pore volume at 50× magnification, according to certain embodiments.

FIG. 25B is an exemplary scanning electron microscopy images showing the microstructure of 30% pore volume at 100× magnification, according to certain embodiments.

FIG. 25C is an exemplary scanning electron microscopy images showing the microstructure of 40% pore volume at 50× magnification, according to certain embodiments.

FIG. 25D is an exemplary scanning electron microscopy images showing the microstructure of 40% pore volume at 100× magnification, according to certain embodiments.

FIG. 25E is an exemplary scanning electron microscopy images showing the microstructure of 50% pore volume at 50× magnification, according to certain embodiments.

FIG. 25F is an exemplary scanning electron microscopy images showing the microstructure of 50% pore volume at 100× magnification, according to certain embodiments.

FIG. 25G is an exemplary graph showing quantitative pore size analysis comparing 30%, 40%, and 50% pore volume scaffold groups with statistical significance indicators, according to certain embodiments.

FIG. 26A is an exemplary graph showing compressive modulus values for bone scaffolds with 30%, 40%, and 50% pore volumes with statistical analysis results, according to certain embodiments.

FIG. 26B is an exemplary graph showing compressive strength values at maximum load for bone scaffolds with 30%, 40%, and 50% pore volumes with statistical analysis results, according to certain embodiments.

FIG. 27 is an exemplary graph showing weight loss percentage over time for bone scaffolds with 30%, 40%, and 50% pore volumes during biodegradation testing, according to certain embodiments.

FIG. 28A is an exemplary fluorescence microscopy images showing viability of human bone marrow-derived stem cells on 30% pore volume bone scaffolds at day 7, according to certain embodiments.

FIG. 28B is an exemplary fluorescence microscopy images showing viability of human bone marrow-derived stem cells on 40% pore volume bone scaffolds at day 7, according to certain embodiments.

FIG. 28C is an exemplary fluorescence microscopy images showing viability of human bone marrow-derived stem cells on 50% pore volume bone scaffolds at day 7, according to certain embodiments.

FIG. 28D is an exemplary graph showing quantitative cell growth rates on 30%, 40%, and 50% pore volume bone scaffolds with statistical analysis results, according to certain embodiments.

FIG. 29A is an exemplary graph showing compressive strength values at maximum load for bone plates with 5 millimeter, 6 millimeter, and 7 millimeter thicknesses with statistical analysis results, according to certain embodiments.

FIG. 29B is an exemplary graph showing compressive modulus values for bone plates with 5 millimeter, 6 millimeter, and 7 millimeter thicknesses with statistical analysis results, according to certain embodiments.

FIG. 30 is an exemplary graph showing weight loss percentage over time for bone plates with 5 millimeter, 6 millimeter, and 7 millimeter thicknesses during biodegradation testing, according to certain embodiments.

FIG. 31A is an exemplary graph showing threshold torque values for bone screws with 6 millimeter, 7 millimeter, and 8 millimeter diameters with statistical analysis results, according to certain embodiments.

FIG. 31B is an exemplary graph showing peak failure torque values for bone screws with 6 millimeter, 7 millimeter, and 8 millimeter diameters with statistical analysis results, according to certain embodiments.

FIG. 31C is an exemplary graph showing peak clamping torque values for bone screws with 6 millimeter, 7 millimeter, and 8 millimeter diameters with statistical analysis results, according to certain embodiments.

FIG. 32 is an exemplary graph showing weight loss percentage over time for bone screws with 6 millimeter, 7 millimeter, and 8 millimeter diameters during biodegradation testing, according to certain embodiments.

FIG. 33 is an exemplary diagram showing the manufacturing process flow from clinical need identification through final product delivery, according to certain embodiments.

FIG. 34 is an exemplary diagram showing integration of the patient-specific bone scaffold, bone plate, and bone screws in relation to the bone fracture site, according to certain embodiments.

FIG. 35 is an exemplary diagram showing a patient-specific bone scaffold with 40% pore volume and views of the scaffold geometry matched to patient anatomy, according to certain embodiments.

FIG. 36 is an exemplary diagram showing a patient-specific bone plate with 7 millimeter thickness and screw hole placement matched to patient anatomy, according to certain embodiments.

FIG. 37 is an exemplary diagram showing patient-specific bone screws with length matched to patient bone diameter and 8 millimeter shaft diameter for optimal fixation, according to certain embodiments.

FIG. 38A is an exemplary engineering drawing of the patient-specific bone scaffold with 40% pore volume, with a ¾ view, according to certain embodiments.

FIG. 38B is an exemplary engineering drawing of the patient-specific bone scaffold with 40% pore volume, with a front view thereof, according to certain embodiments.

FIG. 38C is an exemplary engineering drawing of the patient-specific bone scaffold with 40% pore volume, with a top view thereof, according to certain embodiments.

FIG. 38D is an exemplary engineering drawing of the patient-specific bone scaffold with 40% pore volume, with a right view thereof, according to certain embodiments.

FIG. 39A is an exemplary engineering drawing of the patient-specific bone plate with screw hole placement, with a ¾ view thereof, according to certain embodiments.

FIG. 39B is an exemplary engineering drawing of the patient-specific bone plate with screw hole placement, with a front view thereof, according to certain embodiments.

FIG. 39C is an exemplary engineering drawing of the patient-specific bone plate with screw hole placement, with a right view thereof, according to certain embodiments.

FIG. 39D is an exemplary engineering drawing of the patient-specific bone plate with screw hole placement, with a top view thereof, according to certain embodiments.

FIG. 40A is an exemplary engineering drawing of the first and second patient-specific bone screws, with a ¾ view thereof, according to certain embodiments.

FIG. 40B is an exemplary engineering drawing of the first and second patient-specific bone screws, with a right view thereof, according to certain embodiments.

FIG. 40C is an exemplary engineering drawing of the first and second patient-specific bone screws, with a front view thereof, according to certain embodiments.

FIG. 40D is an exemplary engineering drawing of the first and second patient-specific bone screws, with a right view thereof, according to certain embodiments.

FIG. 40E is an exemplary engineering drawing of the first and second patient-specific bone screws, with a front view thereof, according to certain embodiments.

FIG. 41A is an exemplary engineering drawing of the third and fourth patient-specific bone screws, with a ¾ view thereof, according to certain embodiments.

FIG. 41B is an exemplary engineering drawing of the third and fourth patient-specific bone screws, with a right view thereof, according to certain embodiments.

FIG. 41C is an exemplary engineering drawing of the third and fourth patient-specific bone screws, with a front view thereof, according to certain embodiments.

FIG. 41D is an exemplary engineering drawing of the third and fourth patient-specific bone screws, with a right view thereof, according to certain embodiments.

FIG. 41E is an exemplary engineering drawing of the third and fourth patient-specific bone screws, with a front view thereof, according to certain embodiments.

FIG. 42 is an illustration of a non-limiting example of details of a controller used in a computing system, according to certain embodiments.

FIG. 43 is an exemplary schematic diagram of a data processing system used within the computing system, according to certain embodiments.

FIG. 44 is an exemplary schematic diagram of a processor used with the computing system, according to certain embodiments.

FIG. 45 is an illustration of a non-limiting example of distributed components which may share processing with the controller, according to certain embodiments.

### DETAILED DESCRIPTION

In the drawings, like reference numerals designate identical or corresponding parts throughout the several views. Further, as used herein, the words “a,” “an” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

According to a first aspect, the present disclose relates to a method of repairing a bone fracture in a patient. The method of the present disclosure may be advantageous for providing patient-specific bone substitutes. The method of the present disclosure may be advantageous to overcome the limitations of current treatments discussed above. For example, by utilizing advanced imaging techniques, such as computer tomography (CT) scans, detailed anatomical data can be captured to create precise 3D models of bone defects. Such models may be advantageous to use as a basis for designing personalized bone substitutes that closely match the patient's unique anatomy. Such patient specificity may be advantageous for ensuring an optimal fit and/or reducing the risk of complications associated with non-customized implants. The models may also be used as a basis for constructing the personalized bone substitutes through the use of 3D printing technologies, such as fused deposition modeling (FDM). The method of the present disclosure may further be advantageous for allowing the patient-specific bone substitutes to be fabricated using biocompatible and biodegradable materials with tailored mechanical properties. The use of biodegradable materials in designing and fabricating bone substitutes may offer several advantages. First, they may provide temporary structural support during the healing process, gradually degrading as new bone tissue forms, which may reduce or eliminate the need for implant removal surgeries. Second, biodegradable materials may promote, encourage, or facilitate cell infiltration and vascularization within the scaffold, which may enhance bone regeneration speed and/or quality. Third, the biodegradable scaffolds may serve as platforms for or be augmented by the incorporation of bioactive agents, such as growth factors or stem cells, that can further augment the regenerative potential. This may be advantageous for promoting faster and more efficient healing. The 3D printing process itself may be advantageous for enabling precise control over scaffold geometry, porosity, and interconnectivity. This may allow for customization of the bone substitutes to match the specific anatomical requirements of each patient.

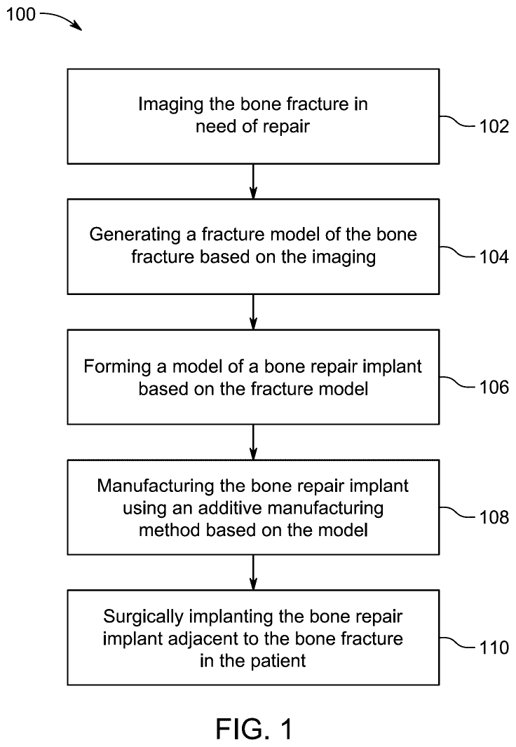

Referring to FIG. 1, illustrated is an exemplary flowchart of a method (as represented by reference numeral 100) of repairing a bone fracture in a patient.

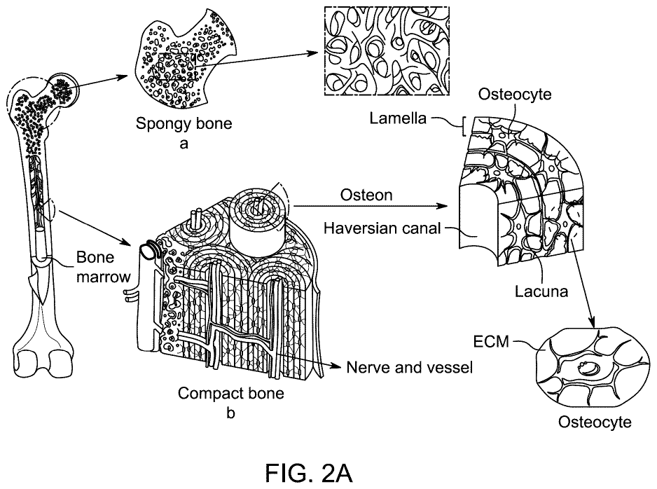

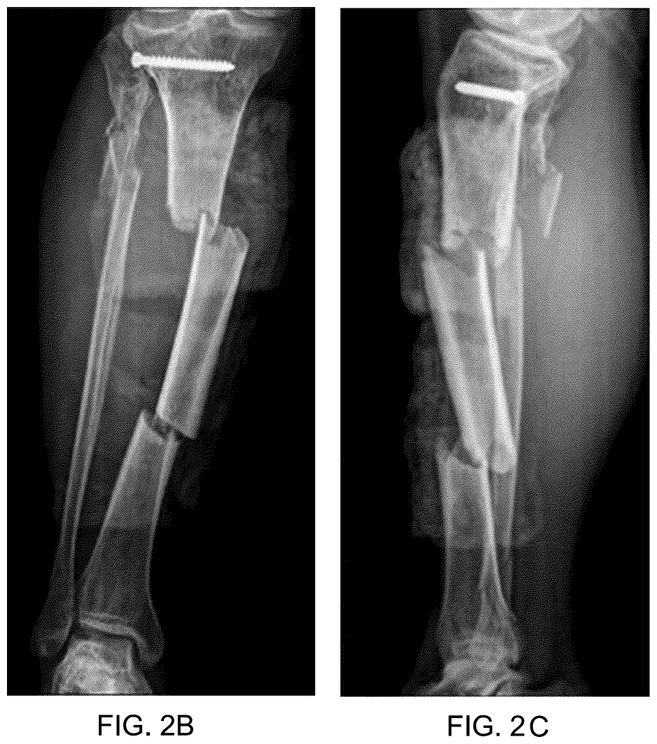

A bone is a dynamic connective tissue comprised of collagens and minerals. It offers vital structural and mechanical support, protects internal organs, and establishes a robust framework. Additionally, it plays a pivotal role in maintaining homeostasis and regulating blood pH levels. It is comprised of two primary structures: cortical and cancellous structures. Cortical bone constitutes the dense and rigid outer layer of bone, providing the majority of its mechanical properties. Cancellous bone, on the other hand, forms the porous and spongy inner layer, characterized by interwoven trabeculae nestled within bone marrow [G. Bouet, et. al., Tissue Eng. Part B Rev., vol. 21, no. 1, pp. 133-156, February 2015, incorporated herein by reference in its entirety]. The cortical bone represents 80% of the skeletal mass of the human body, while the cancellous bone poses only 20% of the skeleton overall mass. The periosteum is the outermost layer that covers all bones and contains nerves and blood vessels. Osteon is the structural unit of the cortical bone that includes osteocytes (bone cells) and a deposit mineralized matrix. The matrix of osteon consists of collagen fibers (the organic component that grants bone its structure) that assist withstand bending or mechanical loads. The collagen fibrils are the fundamental building blocks of collagen fibers and are uniformly threaded with hydroxyapatite (HAP) crystals (the in organic component that grants the bone its strength). FIG. 2A illustrates a cross-sectional view of structural components of cortical and cancellous bone [B. Bisht, et. al., Ann. Biomed. Eng., vol. 49, no. 4, pp. 1128-1150 April 2021, incorporated herein by reference in its entirety].

Bones undergo continuous remodeling while growing and regenerating after injury. The remodeling cycle includes three successive stages: resorption, in which the osteoclasts remove mineralized bone (old bone); reversal, in which the mononuclear cells start appearing on the bone's surface; and formation, in which osteoblasts start forming new bone until the old bone is entirely replaced. Bone remodeling helps to modify bone structures to fulfill the changes in mechanical requirements and repair damage in the bone matrix [D. J. Hadjidakis and I. I. Androulakis, Ann. N. Y. Acad. Sci., vol. 1092, pp. 385-396, December 2006, incorporated herein by reference in its entirety]. Regulated bone remodeling is crucial for the appropriate healing of fractures, skeletal growth, and adaptation to mechanical loads.

A bone defect occurs when there is disturbance in the bone remodeling process and dysfunction in the bone matrix induced by trauma or surgery. Bone remodeling involves continuous modification while growing and regenerating after injury. This remodeling cycle includes three successive stages: resorption, where osteoclasts remove mineralized bone; reversal, where mononuclear cells appear on the bone surface; and formation, where osteoblasts form new bone until complete replacement of old bone. The remodeling process modifies bone structures to accommodate changes in mechanical requirements and repair damage in the bone matrix. In cases of minor bone injuries, known as non-critical segmental bone defects, bone tissue can regenerate and heal through natural mechanisms. However, in cases of major bone injuries, known as critical-sized segmental bone defects where the bone defect exceeds 2 cm in length, natural bone regeneration is not possible, requiring surgical intervention. FIGS. 2B-2C show CT scan images of critical-sized segmental bone fractures.

Two primary mechanisms, endochondral ossification (EO) and intra-membranous ossification (IMO), facilitate bone self-healing after injury. The EO mechanism creates fibrous tissue, granulation tissue, and cartilage tissue by stimulating inflammation between the hematoma at the fracture end, bone marrow cavity, and surroundings. Through this process, osteoblasts replace chondrocytes to form bone tissue. The IMO mechanism causes periosteum thickening and calcification by increasing osteoblast cells in inner and outer periosteum layers, connecting fracture ends. For critical-sized bone defects, repair occurs through EO and IMO mechanisms with placement of tissue-engineered scaffolds at defect sites. These scaffolds comprise three-dimensional structures fabricated from biocompatible materials to fill tissue defects and provide structural support while guiding cell and tissue growth.

At step 102, the method 100 includes imaging the bone fracture in need of repair. In some embodiments, the bone fracture in need of repair includes a proximal bone portion, a missing bone portion, and a distal bone portion. In some embodiments, the bone fracture involves a single bone being broken in at least two places, resulting in three separate bone segments. In critical-sized segmental bone fractures, the missing bone portion exceeds 2 cm in length between the proximal bone portion and the distal bone portion. The proximal bone portion extends from an anatomical joint, such as the hip joint, to a first break point. The missing bone portion represents the gap or defect between the first break point and a second break point. The distal bone portion extends distally from the second break point, such as toward another anatomical joint.

In some embodiments, the proximal bone portion and distal bone portion retain intact bone tissue structure. In some embodiments, the missing bone portion represents a void where bone tissue may be regenerated. In some embodiments, imaging techniques, such as computer tomography scan images, can capture the specific dimensions and geometric relationships between these three distinct portions. For example, the distance from anatomical landmarks on the proximal bone portion to the first break point, and from the second break point on the distal bone portion, may provide useful measurements for modeling the missing bone portion. The boundaries between the proximal bone portion, missing bone portion, and distal bone portion may represent interfaces where bone repair and regeneration may need to occur. These interfaces may greatly benefit from support, proper alignment, and stability during the healing process while enabling biological integration between existing bone tissue and newly formed bone in the missing bone portion region.

In some embodiments, the imaging is at least one selected from the group consisting of computerized tomography, magnetic resonance imaging, and nuclear bone scanning. Computerized tomography (CT) scanning provides detailed anatomical data of bone structure through cross-sectional imaging using X-rays. The computerized tomography scan generates sequential two-dimensional image slices of the bone fracture site, capturing both external bone geometry and internal structural details. The scan images can show clear distinction between bone tissue and surrounding soft tissues, enabling accurate segmentation and three-dimensional reconstruction. Magnetic resonance imaging provides detailed visualization of both bone and soft tissue structures at the fracture site through the use of magnetic fields and radio waves. During magnetic resonance imaging, the patient is positioned within a magnetic resonance imaging machine that generates a strong magnetic field to align hydrogen atoms in water molecules within the body tissues. Radio wave pulses are then applied, causing these atoms to produce signals that are detected and converted into detailed cross-sectional images. The magnetic resonance imaging technique can provide high contrast between different soft tissues, enabling clear differentiation of bone from surrounding muscles, ligaments, and blood vessels. The magnetic resonance imaging data can be processed using the segmentation software to isolate the bone structure and generate three-dimensional models for designing the bone repair implant components. Nuclear bone scanning uses radioactive tracers to identify areas of increased bone metabolism or turnover, which occur at fracture sites. In nuclear bone scanning, a small amount of radioactive material is injected into the patient's bloodstream. The radioactive material accumulates in areas of bone that are actively remodeling or healing. A special camera detects the radiation emitted by the tracers and creates images showing the distribution of the radioactive material. Nuclear bone scanning can detect fractures not readily visible on standard imaging and can provide functional information about bone healing activity. The nuclear bone scan data can supplement anatomical information from computerized tomography or magnetic resonance imaging to guide placement and design of the bone repair implant components. In some embodiments, a combination of anatomical and functional imaging modalities can enable more comprehensive assessment of the bone fracture. Such additional information may be advantageous implant design and/or surgical planning.

In some embodiments, the imaging is computerized tomography (also referred to as “CT”). In some embodiments, computerized tomography scan images are processed using segmentation software to convert the scan data into three-dimensional models of the bone fracture. For example, the computerized tomography scan data can be imported into segmentation software, where the bone structure can be isolated from surrounding tissues through digital filtering and processing. A processed three-dimensional model can be exported from the segmentation software. This model can serve as the basis for designing the patient-specific bone repair implant components. In some embodiments, the three-dimensional model provides precise dimensional and geometrical data of both the fractured bone segments and the missing bone region that needs to be replaced by the bone scaffold component of the bone repair implant. In some embodiments, computer-aided design software uses this three-dimensional model data to generate the design of the bone repair implant components with exact specifications matched to the patient's anatomy.

For processing the three-dimensional models, precise measurements are taken. The distance from anatomical landmarks like the hip joint to the proximal edge of the defect is recorded. The distance between proximal and distal edges of the defect is also measured and documented. These measurements establish baseline parameters for recreating an accurate model of the bone defect. The measurements enable identification of corresponding anatomical segments in the uninjured contralateral femoral bone that align with the defect site. Through this imaging method, a complete three-dimensional representation of the bone fracture and surrounding anatomy is generated. The imaging data provides the foundation for designing patient-specific bone substitutes that precisely match the unique anatomical requirements of each case. The three-dimensional models created through this imaging process serve as templates for subsequent design and fabrication steps in the method 100. The detailed anatomical data obtained through computer tomography scanning and three-dimensional reconstruction enables precise matching of bone substitute geometries to patient anatomy, promoting optimal alignment and integration during healing.

At step 104, the method 100 includes generating a fracture model of the bone fracture based on the imaging (i.e., computer tomography scan images). For this purpose, the three-dimensional STL models exported from the segmentation software are imported into computer aided design software (SolidWorks) for detailed fracture model generation. Within the computer aided design software, horizontal lines are drawn from proximal and distal edges of the defect and extended to the contralateral uninjured bone. The horizontal lines serve as baselines for recreating the bone defect in the contralateral uninjured femoral bone. By extending these lines horizontally to the contralateral uninjured bone, the method 100 identifies segments that align anatomically with the defect in the injured bone. These aligned segments enable accurate modeling of the original bone geometry at the fracture site. The identified segment from the uninjured contralateral bone is then mirrored to match the anatomy of the defect site in the injured bone. This mirroring process creates a three-dimensional model that represents the natural bone geometry that should exist at the fracture site. The fracture model serves as the basis for creating patient-specific bone scaffolds.

The fracture model generation includes determination of precise dimensional requirements. The length from the hip joint to the proximal edge of the defect is measured and recorded. The distance between proximal and distal edges of the defect is documented. These measurements establish geometric parameters that guide design of bone support system components to ensure proper anatomical fit and alignment. The generated fracture model provides a complete three-dimensional representation of both the defect site and target bone geometry. The model captures detailed surface contours, internal structures, and dimensional relationships required for designing bone substitutes that match patient anatomy. This modeling process establishes the geometric foundation for subsequent design and fabrication of the bone support system components in the method 100.

At step 106, the method 100 includes forming a model of a bone repair implant based on the fracture model. The fracture model created from the computer tomography scan images serves as a foundation for designing a bone repair implant that matches the patient's specific anatomy. Using computer aided design software, the three-dimensional geometry of the fracture model is processed to create detailed implant specifications. The forming of the bone repair implant model takes into account the precise measurements obtained from the fracture model, including key anatomical dimensions and surface contours. This ensures the bone repair implant model accurately reflects the geometric requirements of the defect site. The model incorporates features necessary for proper integration with surrounding bone tissue and long-term structural support during healing.

The bone repair implant model is designed to be fabricated using biodegradable materials through three-dimensional printing processes. The model specifies geometric parameters that enable controlled degradation while maintaining required mechanical properties throughout the healing process. These specifications are developed to promote optimal bone tissue regeneration and integration. Through the computer aided design process, the bone repair implant model is refined to ensure manufacturability using fused deposition modeling technology. The bone repair implant model incorporates features that can be accurately reproduced through additive manufacturing processes while maintaining required tolerances for proper anatomical fit and function. This modeling stage establishes the fundamental geometry that will guide subsequent detailed component design and fabrication steps in the method 100.

In aspects of the present disclosure, the bone repair implant comprises a bone scaffold, a support plate, and a screw. Herein, the bone scaffold is designed to fill the void of the missing bone portion between the proximal bone portion and the distal bone portion. The support plate extends across the proximal bone portion, the bone scaffold, and the distal bone portion to provide structural stability during healing. The screw secures the support plate to the bone portions and scaffold, creating a unified support structure. The bone scaffold, support plate, and screw are designed to function as an integrated system to promote bone regeneration while maintaining proper alignment of the proximal bone portion and distal bone portion. The scaffold provides a framework for new bone growth, while the support plate and screw maintain mechanical stability of the repair site. The bone repair implant components are modeled to enable proper geometric interface between each component and with the surrounding bone tissue. The computer aided design software enables modeling of these three components in proper spatial relationship to ensure functional integration. The bone repair implant model specifies how the screw passes through the support plate to secure the assembly, and how the support plate aligns with the bone scaffold to maintain stability during healing. The model establishes the geometric relationships required for the components to work together as a unified bone support system.

In particular, the bone scaffold comprises a network of scaffold members defining an interconnected pore network and has a shape configured to replace a missing bone portion from the bone fracture. Such network of scaffold members creates a highly porous three-dimensional microstructure with interwoven channels throughout the bone scaffold. The interconnected pore network enables enhanced cell infiltration, vascularization, and oxygen delivery throughout the bone scaffold during healing. The interconnected pore network is created through controlled infill patterns during the modeling process. The pore structure allows nutrient flow and waste exchange throughout the scaffold volume while maintaining required mechanical strength. The shape of the bone scaffold is generated from the fracture model to precisely match the geometry of the missing bone portion. Using the contralateral uninjured bone as a reference, the bone scaffold shape mirrors the natural bone anatomy that should exist between the proximal bone portion and distal bone portion. The scaffold shape ensures proper anatomical alignment while providing sufficient surface area for cell attachment and bone ingrowth. The scaffold members form a supportive framework that withstands physiological loads during healing as new bone tissue forms within the pore network. The network of scaffold members and interconnected pores is designed to facilitate bone tissue regeneration through both endochondral ossification and intra-membranous ossification mechanisms. The scaffold structure guides the natural bone healing process while providing temporary mechanical support until sufficient new bone formation occurs.

In an embodiment, the bone scaffold has a pore volume of 25 to 60% of a total volume of the bone scaffold. This pore volume range balances the requirements for mechanical strength with biological function. The pore volume is implemented through controlled infill percentages in the computer aided design and slicing software during the bone scaffold modeling process. The pore volume determines the ratio of void space to solid material within the total volume of the bone scaffold. At pore volumes within this range, the bone scaffold maintains sufficient mechanical strength to withstand physiological loads while providing adequate space for cellular infiltration and new bone formation. The pore volume directly affects the capacity of the bone scaffold to support vascularization and nutrient delivery throughout the scaffold structure. The pore volume of the bone scaffold is achieved through specific geometric arrangements of the scaffold members during the modeling process. When the pore volume is increased within this range, more space is available for cell growth and blood vessel formation, but the overall mechanical strength of the bone scaffold decreases due to reduced solid material content. Conversely, when the pore volume is decreased within this range, the mechanical strength increases but with reduced space for biological processes.

Further, the support plate has an inner surface profile configured to match an outer surface profile of the bone fracture in need of repair in an area covered by the support plate and an outer surface profile which smoothly transitions to match an outer surface profile of the bone fracture in need of repair in an area not covered by the support plate such that the bone repair implant is devoid of sharp edges. Herein, the inner surface profile is generated using Power Surfacing features in computer aided design software to precisely follow the contours of the lateral part of the patient's femoral bone. The support plate model incorporates smooth transitions at all boundaries to eliminate stress concentration points. Where the support plate interfaces with bone surfaces, the inner surface profile maintains continuous contact to distribute loads evenly. The outer surface profile gradually blends with surrounding bone geometry to prevent tissue irritation and ensure patient comfort. The matching surface profiles enable the support plate to maintain proper anatomical alignment while minimizing gaps between the plate and bone surfaces. This close conformity helps distribute mechanical loads across the interface between the support plate and bone tissue. The smooth profile transitions reduce stress concentrations that could lead to mechanical failure or tissue damage during the healing process.

Herein, the support plate is configured to secure the bone scaffold, a proximal bone portion, and a distal bone portion in a repair configuration. The support plate extends across all of these three components to maintain their relative positions during healing. The length of the support plate spans beyond both ends of the fracture position to provide adequate fixation surfaces on the proximal bone portion and distal bone portion. The support plate includes multiple screw holes positioned to enable secure attachment to the bone portions and scaffold. These screw holes are spaced at defined intervals to distribute mechanical loads evenly across the repair site. The spacing and positioning of screw holes enables optimal fixation while avoiding interference with the healing process. The support plate maintains alignment of the bone scaffold between the proximal bone portion and distal bone portion throughout the healing period. This alignment is essential for proper bone regeneration and restoration of anatomical function. The repair configuration established by the support plate ensures stability of all components while the missing bone portion is regenerated through the scaffold structure.

In present embodiments, the support plate is configured to be attached to at least one selected from the group consisting of the proximal bone portion and the distal bone portion using the screw. The support plate includes appropriately positioned screw holes that align with optimal attachment points on either the proximal bone portion or the distal bone portion. The diameter of these screw holes matches the diameter of the screw to ensure secure fixation. The positioning of the screw holes takes into account the bone diameter at the attachment points, which is measured at multiple locations along the bone portions. Such attachment configuration provides mechanical stability while allowing the support plate to maintain proper anatomical alignment during healing.

The support plate is further configured to be attached to both the proximal bone portion using a first screw and the distal bone portion using a second screw. For this purpose, multiple screw holes are positioned at defined intervals along the length of the support plate to accommodate both the first screw and second screw. The spacing between screw holes ranges from 5-10 mm to distribute mechanical loads evenly across the attachment points. In the proposed configuration, the first screw secures the proximal end of the support plate while the second screw secures the distal end, creating a stable bridging structure across the fracture site. Such attachment configuration maintains alignment between the proximal bone portion and distal bone portion throughout the healing process.

The support plate is configured to be further attached to the bone scaffold using a screw. For this purpose, the support plate may include additional screw holes positioned to align with the bone scaffold structure between the proximal and distal attachment points. These screw holes are designed to accommodate screws passing through both the support plate and scaffold material without compromising the structural integrity of either component. The attachment of the support plate to the bone scaffold provides additional stability to the repair site while maintaining proper positioning of the scaffold between the bone portions during regeneration of the missing bone portion.

In the present exemplary embodiments, the support plate has a thickness of 5 to 7 mm. This thickness range provides adequate mechanical strength for load bearing while maintaining a profile that minimizes tissue disruption. The support plate thickness is uniform across its length to ensure consistent load distribution. The thickness dimension is selected to prevent plate deformation under physiological loads while enabling the plate to conform to bone surface contours. When fabricated from polylactic acid, this thickness range enables the support plate to maintain structural integrity throughout the bone healing period.

Further, in the present exemplary embodiments, the screw has a shank diameter of at least 8 mm. The shank diameter is sized to provide sufficient mechanical strength for securing the support plate to bone tissue without failing under torsional loads encountered during placement. This shank diameter enables the screw to withstand the mechanical forces experienced during surgical installation and subsequent physiological loading. The shank diameter is matched to corresponding holes in the support plate to ensure proper fit and secure fixation of all components in the bone repair implant. Further, the screw has a head diameter of at least 9 mm. The head diameter is larger than the shank diameter to create an adequate bearing surface against the support plate. This head diameter provides sufficient surface area for load distribution at the screw-plate interface while preventing the screw head from pulling through the plate material. The head diameter is matched to countersunk regions in the support plate to maintain a smooth outer surface profile of the bone repair implant.

At step 108, the method 100 includes manufacturing the bone repair implant using an additive manufacturing method based on the model. The additive manufacturing method enables direct fabrication from the three-dimensional model without requiring intermediate steps or tooling. The additive manufacturing process precisely controls geometric features during fabrication. For bone scaffolds, the process implements specified pore volumes through controlled infill patterns defined in the slicing software. The manufacturing parameters maintain accurate reproduction of scaffold member networks and interconnected pore structures. For support plates, the process ensures accurate reproduction of surface profiles and screw hole positions. For screws, the process maintains precise control of shank and head diameters. The process parameters are optimized to achieve required dimensional accuracy and surface quality for all bone repair implant components. Thereby, the method 100 enables manufacturing of bone repair implants having precisely controlled geometry, porosity, and interconnectivity to match specific anatomical requirements of each patient.

In particular implementations, the additive manufacturing method is at least one selected from the group consisting of fused deposition modeling, selective laser sintering, and stereolithography. Each of these additive manufacturing methods provides specific capabilities and advantages for fabricating the bone repair implant. The method 100 enables selection of the most appropriate additive manufacturing technique based on specific requirements of the bone repair implant components being fabricated, including dimensional accuracy, surface finish, and mechanical properties needed for the particular application. Fused deposition modeling (FDM) is a widely used additive manufacturing technique that employs the process of melting and heating materials through an extrusion head, effectively converting them into a semi-liquid (molten) state. During the fused deposition modeling process, the material of interest is fed into the printing nozzle. The printing nozzle then heats up the material until it reaches a semi-liquid state. Once reached, the printing nozzle starts extruding the material onto a building platform layer-by-layer until a three-dimensional object is obtained according to an initial computer aided design model. Fused deposition modeling stands out as one of the most cost-effective methods employed for producing three-dimensional structures. Selective laser sintering (SLS) is another additive manufacturing method that involves using a high-powered laser to selectively fuse or sinter powdered particles together, layer by layer, to build the final object according to a computer aided design model. During selective laser sintering, after the first layer is finished, the piston lowers, and a roller adds a second layer of the powdered material over the finished layer's surface, where the new layer bonds to the previous one. Materials used in selective laser sintering are provided in powdered form, with particle size often ranging between 10-15 microns, to facilitate robust particle fusion. It may be understood that the selective laser sintering process proceeds more smoothly when the powders share the same melting point.

Stereolithography is an additive manufacturing method that can produce three-dimensional objects using the process of photocuring. Stereolithography uses an additive manufacturing process which involves the gradual solidification of a photo-crosslinkable resin, layer by layer. With precise control over resolution, stereolithography can yield sophisticated objects with a high degree of mastery over their pore geometries and interconnectivity. The advantage of stereolithography lies in its ability to create patient-specific structures tailored to match the dimensions and geometry of the defect, facilitated by its superior resolution compared to fused deposition modeling and selective laser sintering.

The advancements in the field of 3D-printing technology have significantly contributed to fabricating implants and scaffolds for utilization in biomedical applications, especially for bone reconstruction and regeneration. In tissue engineering, scaffolds are one of the most critical 3D structures that help form a bridge between cells and allow their infiltration. Furthermore, they facilitate the formation of bone tissue by providing an extracellular matrix space [H. N. Chia and B. M. Wu, “Recent advances in 3D printing of biomaterials,” J. Biol. Eng., vol. 9, no. 1, p. 4, March 2015, incorporated herein by reference in its entirety]. Nowadays, the use of 3D printing technology in developing bone scaffolds has increased. With this technology, almost any material can be used to produce 3D scaffolds with unique geometries [C. Wang et al., “3D printing of bone tissue engineering scaffolds,” Bioact. Mater., vol. 5, no. 1, pp. 82-91, March 2020, incorporated herein by reference in its entirety]. It retains considerable advantages in the field of bone tissue engineering due to its ability to offer high production capacity, cost and time-effectiveness, and precise control over porosity and interconnectivity. Moreover, a major advantage of 3D printing its ability to produce patient-specific bone scaffolds made of micro or nano-scale biomaterial that match the patient's needs and defect geometry, which ensures better graft-tissue integration and enhanced bone growth. Since allografts and autografts are limited by the geometry of the site from where they are harvested, this technology can overcome this constraint by producing bone grafts that are tailored to defect geometries; thereby ensuring better outcomes.

3D printing is an additive manufacturing (AM) technology that can produce 3D tissue scaffolds by depositing biomaterial layer-by-layer [L. Cheng et al., “3D Printing of Micro- and Nanoscale Bone Substitutes: A Review on Technical and Translational Perspectives,” Int. J. Nanomedicine, vol. 16, pp. 4289-4319 June 2021, incorporated herein by reference in its entirety]. To facilitate the fabrication of bone tissue engineering scaffolds using the 3D printing technology, a 3D digital model of the fractured bone is created from a computer tomography (CT) scan [P. Honigmann, N. Sharma, B. Okolo, U. Popp, B. Msallem, and F. M. Thieringer, “Patient-Specific Surgical Implants Made of 3D Printed PEEK: Material, Technology, and Scope of Surgical Application,” BioMed Res. Int., vol. 2018, p. 4520636, 2018, incorporated herein by reference in its entirety]. This model is then imported into a 3D printer, ultimately resulting in the production of a 3D scaffold that mimics the lost bone's overall geometry. It is crucial to note that specific characteristics may need to be achieved in tissue engineering scaffolds for optimal and enhanced tissue regeneration. First, precise replication of the defect anatomy may need to be achieved in the scaffold's design to ensure precise fit and seamless integration with existing tissue. Additionally, the material used needs to be biocompatible to ensure that the scaffold does not provoke an immune response. Moreover, the material used needs to may need to be biodegradable to allow the scaffold to gradually degrade as natural tissue forms, thereby, eliminating the need for secondary removal surgeries. In the case of bone tissue engineering scaffolds, the scaffold may also need to exhibit robust mechanical properties to provide adequate support and stability. Finally, the scaffold should contain high porosity to facilitate nutrient and oxygen diffusion, which is crucial for cell survival and tissue growth. All of these attributes can easily be achieved through the application of 3D printing technology, and are often hardly achieved using traditional fabrication methods described above.

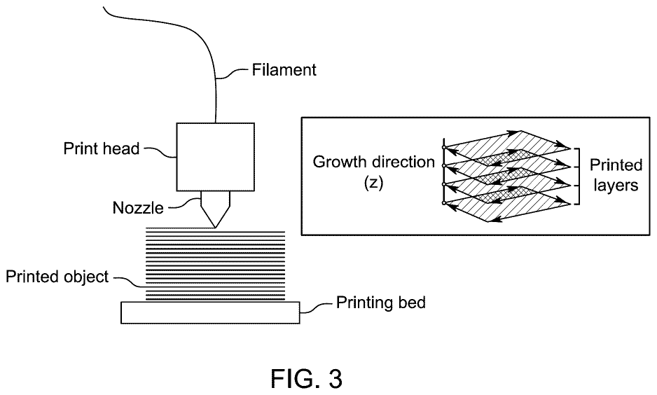

Fused Deposition Modeling (FDM) is a widely used 3D printing technique that employs the process of melting and heating materials through an extrusion head, effectively converting them into a semi-liquid (molten) state. FIG. 3 illustrates a schematic of an FDM 3D Printer [A. R. C. Duarte, et. al., Int. J. Pharm., vol. 332, no. 1-2, pp. 132-139, March 2007, incorporated herein by reference in its entirety]. This technology stands out as one of the most cost-effective methods employed for producing 3D structures [Q. Zhang et al., “3D printing method for bone tissue engineering scaffold,” Med. Nov. Technol. Devices, vol. 17, p. 100205, March 2023, incorporated herein by reference in its entirety]. During the process, the material of interest (often thermoplastics) is fed into the printing nozzle. The printing nozzle then heats up the thermoplastic until it reaches a semi-liquid state. Ones reached, the printing nozzle stars extruding the material onto a building platform layer-by-layer until a 3D object is obtained according to an initial CAD design. Due to its efficiency in rapidly producing 3D objects with accurate geometries, FDM 3D printing technology has been employed in the production of 3D tissue scaffolds to replicate the natural tissue structure. 3D scaffolds with precise control over the scaffold's geometries and porosity can now be produced using this technology. To achieve this, it is crucial that the molten material maintains high temperatures and viscosity to ensure quick fusion with the preceding solid layer and easy flow through the extrusion nozzle. This rigorous process also guarantees accuracy in the scaffold's overall geometries. Some limitations of this technique include its inability to incorporate living cells during the printing process due to the high printing temperatures. Additionally, the heating process of the material may affect the material's mechanical properties, subsequently influencing the overall scaffold integrity. These challenges can be solved by selecting a suitable material for FDM printing, adjusting the cooling mechanisms, and controlling the degree of porosity to enhance mechanical strength. Moreover, the incorporation of living cells into the scaffold can be accomplished after the 3D-printed structure has been produced and properly cooled [S. Rouf, A. Raina, M. Irfan Ul Haq, N. Naveed, S. Jeganmohan, and A. Farzana Kichloo, “3D printed parts and mechanical properties: Influencing parameters, sustainability aspects, global market scenario, challenges and applications,” Adv. Ind. Eng. Polym. Res., vol. 5, no. 3, pp. 143-158, July 2022, incorporated herein by reference in its entirety].

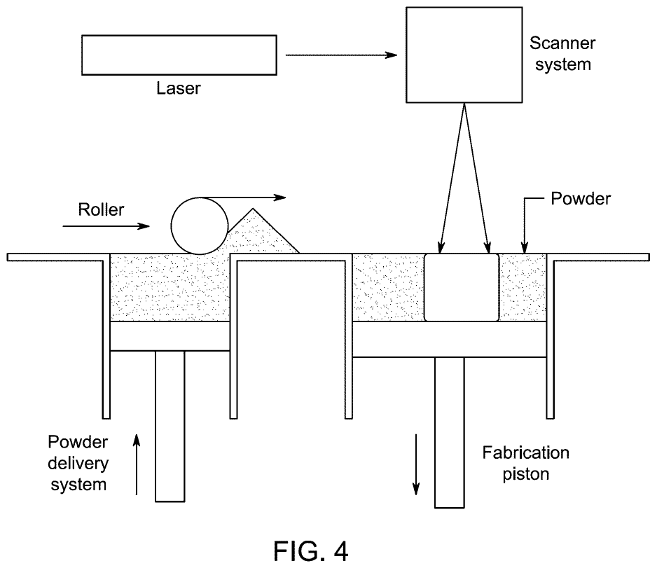

Selective laser sintering (SLS) is another type of 3D printing technology that was developed in 1989. FIG. 4 illustrates a schematic of a SLS 3D Printer [J. L. Brown, et. al., “Chapter II.6.7-Bone Tissue Engineering,” in Biomaterials Science (Third Edition), B. D. Ratner, A. S. Hoffman, F. J. Schoen, and J. E. Lemons, Eds., Academic Press, 2013, pp. 1194-1214, incorporated herein by reference in its entirety]. This process involves using a high-powered laser to selectively fuse or sinter powdered particles together, layer by layer, to build the final object according to a CAD design. This technology is mostly industrial and is ideal for complex engineering, including product development and rapid prototyping in a wide range of medical industries. Materials used in SLS can range from metals, nylon, glass, and ceramic to polyesters and even mixture of materials. Materials used in SLS printing may need to be in powdered form (with particle size often ranging between 10-15 microns) to facilitate robust particle fusion. During printing, and after the first layer is finished, the piston lowers, and a roller adds a second layer of the powdered material over the finished layer's surface, where the new layer bonds to the previous one. This process proceeds more smoothly when the powders share the same melting point. This suggests that objects created from a single material will be more readily produced compared to those made from a mixture of materials. SLS 3D printing is suitable for making very complex designs such as scaffolds with a porous structure. Using this technology, designers can explore and unlock different design possibilities that are may not be possible with traditional techniques. A unique feature of SLS printing in terms of design is that the designer can combine complex assemblies that require multiple parts into a single part. Another advantage is that it is compatible with a wide range of materials, thereby, fulfilling different needs. Finally, this technology does not require dedicated support structures as in FDM and SLA, which saves time and material. However, a drawback of SLS printing is the high temperature it requires to facilitate the fusion of the powder particles, which leads to the destruction of live cells and growth factors during the process. As a result, it is not feasible to print parts with live cells using this printing technique similar to the FDM printing. Another drawback is the high cost associated with SLS technology, which is mainly attributed to the high prices of high-density diodes. This has resulted in a limiting the application for this technology. SLS printing is unique. However, it requires extensive knowledge and understanding for better use.

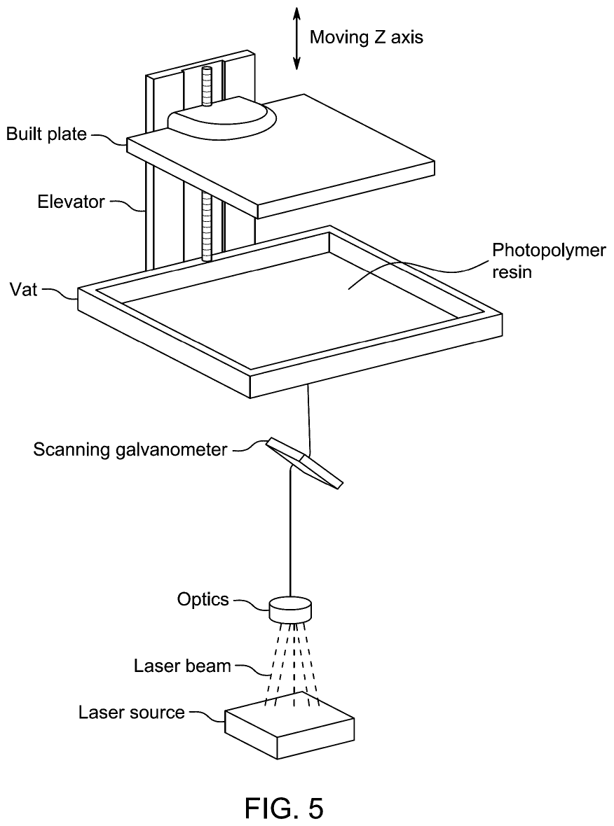

Stereolithography (SLA) is a 3D printing technology that can produce 3D objects using the process of photocuring. SLA uses an AM process, which involves the gradual solidification of a photo-crosslinkable resin, layer by layer. FIG. 5 illustrates a schematic of an SLA 3D Printer [J. Hong, et. al., “Cell-Electrospinning and Its Application for Tissue Engineering”, Int. J. Mol. Sci., vol 20, no. 24, pp 6208 December 2019, incorporated herein by reference in its entirety]. With precise control over resolution, this method can yield sophisticated objects with a high degree of mastery over their pore geometries and interconnectivity. These factors enhance effective cell infiltration and significantly enhance the overall strength of the scaffold. The advantage of the SLA technique lies in its ability to create patient-specific structures tailored to match the dimensions and geometry of the defect. This is facilitated by its superior resolution compared to FDM and SLS printing [Y. Xu, et. al., “Unraveling of Advances in 3D-Printed Polymer-Based Bone Scaffolds.”, Polymers, vol. 14, no. 3, January 2022, incorporated herein by reference in its entirety]. However, a drawback is that it does not allow for the incorporation of living cells and growth factors with the material during the printing process due to the need for post-fabrication processing steps [E. S. Bishop et al., “3-D bioprinting technologies in tissue engineering and regenerative medicine: Current and future trends,” Genes Dis., vol. 4, no. 4, pp. 185-195, November 2017, incorporated herein by reference in its entirety].

Further, in embodiments of the present disclosure, the bone repair implant is constructed of a biodegradable material which is at least one selected from the group consisting of polylactic acid, polyglycolic acid, and poly lactic acid glycolic acid. The biodegradable material provides temporal structural support and gradual degradation during the healing process. The degradation capacity of the biodegradable material allows the production of empty spaces over time that promote enhanced cell infiltration and vascularization throughout the bone repair implant. This infiltration of blood vessels within the bone repair implant due to the degradation encourages the formation of new bone and offers adequate oxygen for regenerated tissues. Specifically, as the bone repair implant starts to degrade, bone tissue extends to the implant's interior, which regulates the regenerative environment. Meanwhile, the mechanical properties of the bone repair implant decrease gradually, and the applied load transitions from the implant to new bone tissue, avoiding stress shield impact. The utilization of these biodegradable materials eliminates the need for secondary removal surgeries, reducing both injury risk and financial burden to the patient.

In particular, a biomaterial is any substance, natural or synthetic, that is intended to be introduced into the body to interact with biological systems. The overall properties of the 3D printed scaffolds can be fine-tuned based on the materials used. For bone tissue engineering, the mechanical, physical, and biochemical properties of the selected material should mimic that of the natural bone to prevent graft structural and biological failure. Additionally, the biomaterial has to be biocompatible to ensure that the scaffold does not provoke an immune response and prevent rejection. Moreover, the biomaterial should be biodegradable to allow the gradual degradation as natural tissue forms, eliminating the need for secondary removal surgeries. Also, the degradation rate of the biomaterial should be proportional to the rate of new bone formation to establish a balanced relationship between degradation and new tissue formation [T. Ghassemi, A. Shahroodi, M. H. Ebrahimzadeh, A. Mousavian, J. Movaffagh, and A. Moradi, “Current Concepts in Scaffolding for Bone Tissue Engineering,” Arch. Bone Jt. Surg., vol. 6, no. 2, pp. 90-99, March 2018, incorporated herein by reference in its entirety].

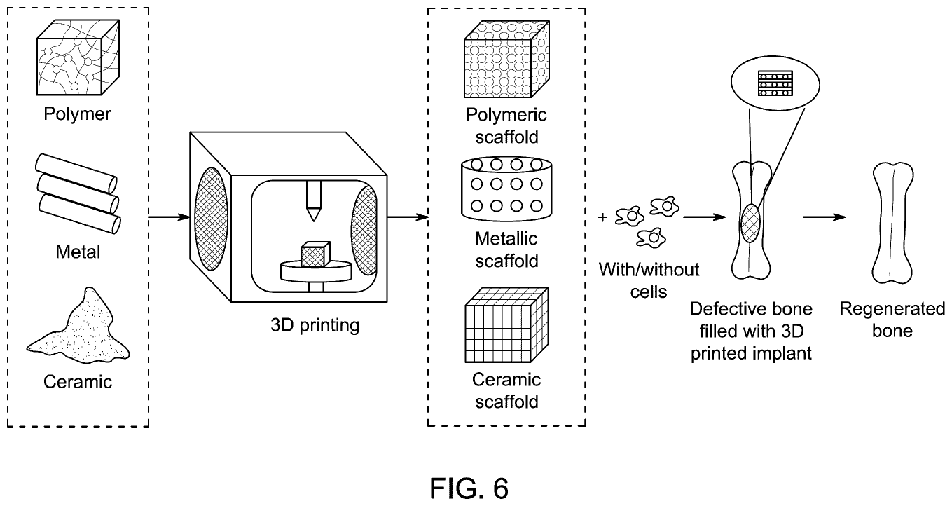

To date, several biomaterials have been developed and used for 3D printing of bone tissue engineering scaffolds including, metals, polymers, and ceramics. FIG. 6 provides a graphical illustration of how different biomaterials are combined with 3D Printing technology to form scaffolds [T. Kim, et. al., Eng. Regen., vol. 1, pp. 6-18, January 2020, incorporated herein by reference in its entirety]. A comprehensive understanding of the characteristics of these materials is crucial to ensure their appropriate utility in the field of bone tissue engineering. Table 1 (below) shows the characteristics of the biomaterials that has been used in bone tissue engineering.

TABLE 1
Characteristics of natural bone tissue compared
with degradable and non-degradable biomaterials

| | | | | | | |
|---|---|---|---|---|---|---|
| | | | | | | Loss of |
| | Compressive | Tensile | Young's | | Degradation | Total |
| | Strength | Strength | Modulus | Elongation | Time | Strength |
| Material Type | (MPa) | (MPa) | (GPa) | (%) | (Months) | (Months) |

Bone

| | | | | | | |
|---|---|---|---|---|---|---|
| Human | 131-224 | 35-283 | 17-20 | 1.07-2.10 | NBR | None |
| Cortical | | | | | | |
| Human | 5-10 | 1.5-38 | 0.05-0.1 | 0.5-3 | NBR | 0.5-1 |
| Cancellous | | | | | | |

Degradable

| | | | | | | |
|---|---|---|---|---|---|---|
| Collagen | 0.5-1 | 50-150 | 0.002-5 | 3 | 2-4 | 1-4 |
| Chitosan | 1.7-3.4 | 35-75 | 2-18 | 1-2 | 4-6 | <3 |
| D, L(PLA) | 15-25 | 90-103 | 1.9 | 3-10 | 12-16 | 4 |
| PLGA | 40-55 | 55-80 | 1.4-2.8 | 3-10 | 1-12 | 1 |
| PCL | 20-40 | 10-35 | 0.4-0.6 | 300-500 | >24 | >6 |
| Hydroxyapatite | 500-1000 | 40-200 | 80-110 | 0.5-1 | >24 | >12 |
| TCP | 154 | 25-80 | 60-75 | 1-2 | >24 | 1-6 |

Non-Degradable

| | | | | | | |
|---|---|---|---|---|---|---|
| Titanium alloy | 900 | 900-1000 | 110-127 | 10-15 | No | None |
| Stainless steel | 500-1000 | 460-1700 | 180-205 | 10-40 | No | None |

* NBR: Natural bone remodeling; PLA: Poly lactic acid; PLGA: Poly lactic glycolic acid; PCL: Poly caprolactone; TCP: Tricalcium phosphate.

There are many biocompatible metallic materials used in bone tissue engineering. These materials hold high fracture stiffness and mechanical strength and are mainly used in dental implants and orthopedics to provide structural and mechanical support. The most commonly used metallic materials are stainless steel (ASTM F138), Co-based alloys (ASTM F75 and ASTM F799), and titanium alloys (Ti-6Al-4V). Each of these materials come with its own set of advantages and disadvantages. For instance, stainless steel is known for its strength and corrosion resistance, is a cost-effective choice, but its higher modulus of elasticity compared to natural bone may lead to stress shielding. Co-based alloys provide excellent strength, wear resistance, and corrosion resistance, marking them suitable for applications where high strength due to load bearing is required. However, similar to stainless steel, co-based alloys also have a higher modulus of elasticity. Titanium alloys, like Ti-6Al-4V, are widely used in orthopedic implants due to their balanced combination of strength, low density (similar to bone), and corrosion resistance. It is noteworthy that titanium alloys possess a lower modulus of elasticity, which reduces the risk of stress shielding. However, they are more expensive compared to both stainless steel and co-based alloys. Regardless of their high cost, titanium alloys offer a better choice for bone implants in tissue engineering due to their superior biocompatibility and mechanical properties compared to both stainless steel and co-based alloys [Bisht et al.; K. Alvarez and H. Nakajima, “Metallic Scaffolds for Bone Regeneration,” Materials, vol. 2, no. 3, pp. 790-832, July 2009, incorporated herein by reference in its entirety].

Polymers are materials composed of repeating monomers (can be the same or different) that bond covalently. The structure of these materials can be amorphous or crystalline with linear, branched, or cross-linked chains [Z. Sheikh, S. Najeeb, Z. Khurshid, V. Verma, H. Rashid, and M. Glogauer, “Biodegradable Materials for Bone Repair and Tissue Engineering Applications,” Materials, vol. 8, no. 9, pp. 5744-5794 August 2015, incorporated herein by reference in its entirety]. There are two types of polymers: natural and synthetic polymers. Natural polymers, including collagen, chitosan, silk, and alginate, usually retain bio-functional properties that ensure bioactivity and natural remodeling. However, their immune response, uncontrollable degradation rate, and inefficient mechanical strength restrict their use in bone tissue engineering [F. Donnaloja, E. Jacchetti, M. Soncini, and M. T. Raimondi, “Natural and Synthetic Polymers for Bone Scaffolds Optimization,” Polymers, vol. 12, no. 4, p. 905, April 2020, incorporated herein by reference in its entirety]. On the other hand, utilizing synthetic polymers provides more reproducible, tailored, and predictable scaffold characteristics yet has low osteoconductivity and bioactivity. Polylactic acid (PLA), poly-caprolactone (PCL), and polylactic-co-glycolide (PLGA) are the most commonly used synthetic polymers for bone tissue engineering. These polymers have proved their biocompatibility, controlled degradation rate, and nontoxic products to surrounding tissues. In addition, their mechanical properties can be enhanced by manipulating the design and synthesis parameters of the scaffold [S. Wei, et. al., Mil. Med. Res., vol. 7, p. 54, November 2020, incorporated herein by reference in its entirety].

In an embodiment, the biodegradable material comprises polylactic acid. Polylactic acid (PLA) consists of a sequence of lactic acid monomers and exists in two enantiomeric structures: the D- and L-optical isomers. Polylactic acid is obtained through the fermentation of renewable structures of sugar products, making it eco-friendly and suitable for utilization inside the human body. Polylactic acid exhibits high mechanical strength, good biodegradability, biocompatibility, and high processability using a wide range of fabrication techniques. Polylactic acid undergoes bulk degradation and takes up to 12-16 months to completely degrade. This degradation timeline makes polylactic acid particularly suitable for bone repair since it retains good mechanical properties for a prolonged time post-transplantation and exhibits a degradation profile that is proportional to the formation of new bone.

Among the synthetic materials discussed above, PLA emerges as a highly favorable choice for bone tissue engineering. It is well documented biocompatibility, adequate mechanical properties, and suitable biodegradation rate, matching that that rate of new bone formation, make it an excellent option. PLA consists of a sequence of lactic acid monomers and exists in two enantiomeric structures: the D- and L-optical isomers. This material is considered eco-friendly and can be utilized inside the human body since it is obtained through the fermentation of renewable structures of sugar products. It holds the advantage of high mechanical strength, good biodegradability, biocompatibility, and high processability using a wide range of fabrication techniques. Although PLA is used in a wide range of applications, it retains some disadvantages, including its brittle nature and insufficient thermal stability. However, different methods were employed to overcome these limitations by combining PLA with other polymers, such as PCL and polyethylene oxide (PEO). A study conducted in 2015 found that combining PLA with PCL in tissue-engineering grafts yields more promising outcomes in bone repair [X. Xu, J. Yang, L. Ding, and J. Li, “Bone morphogenetic protein-2-encapsulated grafted-poly-lactic acid-polycaprolactone nanoparticles promote bone repair,” Cell Biochem. Biophys., vol. 71, no. 1, pp. 215-225, January 2015, incorporated herein by reference in its entirety]. Also, PLA can be integrated with biodegradable ceramics such as hydroxyapatite (HA) to produce scaffolds.

According to Zhang et al., the general performance of the designed porous scaffold was ideal when the mass ratio of PLA/HA was 8:2 [Y. Zhang et al., “Preparation of poly(lactic acid)/sintered hydroxyapatite composite biomaterial by supercritical CO₂,” Biomed. Mater. Eng., vol. 29, no. 1, pp. 67-79, 2018, incorporated herein by reference in its entirety].

Polyglycolic acid (PGA) is another biodegradable material option for constructing the bone repair implant. Polyglycolic acid exhibits a faster degradation rate compared to polylactic acid, completely degrading within approximately 4 weeks. The rapid degradation rate of polyglycolic acid can be advantageous in applications where faster absorption is desired. PCL is another synthetic polymer, composed of hexanoate repeating units. Although biocompatible and biodegradable, many researchers discovered that this material has insufficient mechanical properties and a slow degradation rate; hence, it cannot be considered an ideal material for bone repair. However, many studies have shown that combining this material with bioceramics can improve its overall characteristics. Poly lactic acid glycolic acid, specifically known as polylactic-co-glycolide (PLGA), is a copolymer composed of both polylactic acid (PLA) and polyglycolic acid (PGA). PLGA is widely used in many medical applications since it retains desirable properties such as good mechanical strength, biocompatibility, and regulated degradation rate. PLGA completely degrades within approximately 4 months. Further, the degradation rate of PLGA can be controlled by adjusting the ratio of PLA and PGA in the copolymer. Despite the overhead promising aspects, PLGA has poor osteoinductivity and hydrophilicity, which may restrict its use in bone tissue engineering [M. Mehrasa, M. A. Asadollahi, K. Ghaedi, H. Salehi, and A. Arpanaei, “Electrospun aligned PLGA and PLGA/gelatin nanofibers embedded with silica nanoparticles for tissue engineering,” Int. J. Biol. Macromol., vol. 79, pp. 687-695, August 2015, incorporated herein by reference in its entirety].

Ceramics, such as tricalcium phosphate, hydroxyapatite, and dicalcium phosphate, are a class of biocompatible materials that are used to create scaffolds or implants for supporting the growth of new bone tissues. These ceramics are typically made of inorganic compounds, such as calcium phosphate, and are designed to mimic the natural properties of bone tissue. In recent years, ceramics have been frequently used in the field of tissue repair and replacement, specifically bone tissue engineering, due to their biomimetic chemical structure, biocompatibility, biodegradability, and bioactivity [W. Habraken, P. Habibovic, M. Epple, and M. Bohner, “Calcium phosphates in biomedical applications: materials for the future?,” Mater. Today, vol. 19, no. 2, pp. 69-87, March 2016, incorporated herein by reference in its entirety]. Additionally, they have high mechanical strength and can provide the necessary structural support for regenerating tissues [Z. Sheikh, M. Geffers, T. Christel, J. E. Barralet, and U. Gbureck, “Chelate setting of alkali ion substituted calcium phosphates,” Ceram. Int., vol. 41, no. 8, p. 10010, 2015, incorporated herein by reference in its entirety]. However, ceramics have some drawbacks, such as brittleness, high rigidity, and poor fracture stiffness, which may limit their benefit in critical-size bone fractures. Yet, to address these limitations, ceramics can be combined with other biomaterials such as PCL, PLGA, pr PLA [M. S. Hasan, I. Ahmed, A. J. Parsons, C. D. Rudd, G. S. Walker, and C. A. Scotchford, “Investigating the use of coupling agents to improve the interfacial properties between a resorbable phosphate glass and polylactic acid matrix,” J. Biomater. Appl., vol. 28, no. 3, pp. 354-366, September 2013, incorporated herein by reference in its entirety].

The importance of biodegradable materials in regenerative engineering and bone repair cannot be underestimated [Z. Sheikh, C. Sima, and M. Glogauer, “Bone Replacement Materials and Techniques Used for Achieving Vertical Alveolar Bone Augmentation,” Materials, vol. 8, no. 6, pp. 2953-2993 May 2015, incorporated herein by reference in its entirety]. They provide temporal structural support and gradual degradation [L. L. Hench and J. M. Polak, “Third-generation biomedical materials”, Science, vol. 295, no. 5557, pp 1014-7, February 2022, incorporated herein by reference in its entirety]. Moreover, the degradation capacity of biomaterials allows the production of empty spaces over time that promote enhanced cell infiltration and vascularization. This infiltration of blood vessels within the scaffold due to the scaffold's degradation encourages the formation of new bone and offers adequate oxygen for regenerated tissues. As the scaffold starts to degrade, bone tissue extends to the scaffold's interior, which will regulate the regenerative environment. Hence, it adapts to the maturation of bone tissue. Meanwhile, the scaffold's mechanical properties will decrease, and the applied load will move from the scaffold to new bone tissue, avoiding stress shield impact [C. Chanlalit, D. R. Shukla, J. S. Fitzsimmons, K.-N. An, and S. W. O'Driscoll, “Stress shielding around radial head prostheses,” J. Hand Surg., vol. 37, no. 10, pp. 2118-2125 October 2012, incorporated herein by reference in its entirety]. Therefore, utilizing biodegradable biomaterials eliminates the injury and financial burden caused by a second surgery.

It has been marked that segmental bone fractures require a healing time of 10-18 months despite the differences in the type of bone and defect size [G. K. Naughton, W. R. Tolbert, and T. M. Grillot, “Emerging developments in tissue engineering and cell technology,” Tissue Eng., vol. 1, no. 2, pp. 211-219, 1995, incorporated herein by reference in its entirety]. Typically, polymers undergo bulk degradation. For example, D, L (PLA) and L (PLA) are biodegradable polymers used for fracture fixation and degrades within 12-16 months. Hence, this makes these biodegradable polymers a successful choice for bone repair since they retain good mechanical properties for a prolonged time post-transplantation as well as exhibit a degradation profile that is proportional to the formation of new bone; meaning, as the new bone fully forms, the polymers will completely degrade.

Besides the aforementioned advantages of utilizing biodegradable materials in bone grafts, they can easily be processed into 3D structures with patient-specific geometries and properties via the 3D printing technology; thanks to their unique thermal properties [I. Zein, D. W. Hutmacher, K. C. Tan, and S. H. Teoh, “Fused deposition modeling of novel scaffold architectures for tissue engineering applications,” Biomaterials, vol. 23, no. 4, pp. 1169-1185 February 2002, incorporated herein by reference in its entirety]. This highlights the advantage of combining biodegradable materials with 3D printing technology in the production of bone tissue engineering scaffolds that align with the patient's unique anatomy. These scaffolds are porous, biocompatible, biodegradable, and exhibit robust mechanical properties, all of which are expected to significantly enhance bone repair.

At step 110, the method 100 includes surgically implanting the bone repair implant adjacent to the bone fracture in the patient. The surgical implantation enables placement of the bone repair implant to properly align with and support the bone fracture during the healing process. During implantation, the bone scaffold component of the bone repair implant is positioned to fill the void of the missing bone portion between the proximal bone portion and the distal bone portion. The positioning of the bone scaffold ensures proper anatomical alignment while providing sufficient surface area for cell attachment and bone ingrowth. The surgical implantation establishes a repair configuration where the bone scaffold, support plate, and screws work as an integrated system to promote bone regeneration while maintaining proper alignment of the proximal bone portion and distal bone portion. The scaffold provides a framework for new bone growth, while the support plate and screws maintain mechanical stability of the repair site. The repair configuration established by the surgical implantation ensures stability of all components while the missing bone portion is regenerated through the scaffold structure.

Specifically, the support plate component of the bone repair implant is implanted to extend across the proximal bone portion, the bone scaffold, and the distal bone portion to provide structural stability during healing. The support plate is surgically positioned such that its inner surface profile maintains continuous contact with the outer surface profile of the bone fracture in the area covered by the support plate. This close conformity helps distribute mechanical loads across the interface between the support plate and bone tissue. The outer surface profile of the support plate is positioned to smoothly transition to match the outer surface profile of the bone fracture in areas not covered by the support plate, eliminating sharp edges that could cause tissue irritation. The screw component of the bone repair implant is surgically inserted to secure the support plate to the bone portions and scaffold, creating a unified support structure. The screws are implanted through appropriately positioned screw holes in the support plate that align with optimal attachment points on either the proximal bone portion or the distal bone portion. During implantation, the screws are positioned such that the diameter of the screw holes matches the diameter of the screws to ensure secure fixation.

In accordance with various embodiments of the present disclosure, the patient-specific geometry of the bone repair implant advantageously provides precise anatomical matching between the implant and the patient's bone structure. The method 100 enables fabrication of bone repair implants having geometries precisely matched to individual patient anatomy based on the three-dimensional models generated from computerized tomography scan data. Such precise geometric matching substantially improves implant fit and alignment during surgical implantation compared to conventional non-customized implants. The patient-specific geometry also facilitates proper anatomical positioning of the bone repair implant components relative to surrounding bone tissue. The customized geometric features of the bone scaffold enable precise filling of the missing bone portion void. The patient-specific surface profiles of the support plate provide optimal conformity with bone surfaces. The customized lengths of the screws match patient bone diameters for enhanced fixation.

The biodegradable nature of the implant materials provides controlled degradation synchronized with new bone formation. The degradation profile of the selected biodegradable material substantially matches natural bone healing timeframes. As new bone tissue progressively forms within the scaffold structure, the implant material gradually degrades and is absorbed by the body. This synchronized degradation-regeneration process advantageously eliminates the need for secondary surgical procedures to remove implant components. In particular embodiments, the method 100 provides a treatment approach that reduces surgical complications compared to conventional implants requiring removal surgery. The elimination of removal surgery decreases risks associated with additional surgical procedures, such as infection, bleeding, and anesthesia complications. Patient recovery time and healthcare costs are also reduced by avoiding secondary procedures.

Further embodiments of the present disclosure enable the bone repair implant to maintain sufficient mechanical properties during initial healing stages while allowing controlled degradation as bone regeneration progresses. The degradation rate of the implant materials is selected to provide adequate structural support until sufficient new bone formation occurs. This ensures stable fixation during critical healing periods while enabling gradual transfer of mechanical loads to regenerated bone tissue.

In an embodiment, the method 100 further comprises calculating a mechanical stress response of the model of a bone repair implant to an expected mechanical stress. In particular implementations, finite element analysis is performed to assess the mechanical integrity of the bone repair implant components in their capacity to withstand mechanical loads. This evaluation is crucial, as the bone repair implant will be placed in a load-bearing anatomical site. The finite element analysis is performed using computer aided design software wherein the boundaries are defined, including the constraint surfaces and the areas where physiologically relevant forces would be applied to the three-dimensional models. The mechanical stress analysis ensures the bone scaffold maintains sufficient mechanical strength to withstand physiological loads during healing as new bone tissue forms within the pore network. For support plates, the analysis verifies the chosen thickness can adequately distribute mechanical loads while maintaining a profile that minimizes tissue disruption. For screws, the analysis confirms the selected diameter can withstand the mechanical forces experienced during surgical installation and subsequent physiological loading.

Herein, the expected mechanical stress comprises at least one selected from the group consisting of a compressive stress, a torsional stress, and a bending stress. For evaluating compressive stress, the bone scaffold components are placed perpendicularly between two plates in the analysis software and compression load is applied until complete failure is predicted. The generated force and displacement data are used to calculate the compressive strength and compressive modulus. For evaluating torsional stress, particularly for the screw components, the analysis simulates positioning between two grips (one fixed and one rotating grip) with torsion force applied from one end until complete failure is predicted. The threshold torque, peak failure torque, and peak clamping torque values are calculated. For evaluating bending stress, particularly for the support plate components, a four-point bending analysis is conducted wherein the plates are simulated as being placed horizontally onto two supporting beams spaced at a defined distance, with loading forces applied until failure is predicted.

In present implementations, the expected mechanical stress is based on a location of the bone fracture in need of repair. Different anatomical locations experience different types and magnitudes of mechanical loads during normal physiological function. For instance, when the bone fracture is located in a femoral bone, the analysis applies forces equivalent to average human body weight (approximately 800 newtons) to evaluate the capacity of the bone repair implant to withstand typical load-bearing stresses at this location. The mechanical stress analysis takes into account the specific dimensional requirements of the location, including the distance from anatomical landmarks to the fracture site, and the geometric relationships between the proximal bone portion, missing bone portion, and distal bone portion at the specific anatomical location.

The method 100 further comprises, when the mechanical stress response of the model of the bone repair implant to the expected mechanical stress is calculated to be unacceptable, adjusting the model of the bone repair implant to produce an acceptable mechanical stress response. An unacceptable mechanical stress response occurs when the calculated stress values exceed the maximum yield value of the materials used, indicating potential failure under physiological loads. The adjustment process involves iterative modifications to various parameters of the bone repair implant components until acceptable stress responses are achieved. Each adjustment is followed by recalculation of mechanical stress response until acceptable values are achieved. An acceptable mechanical stress response is indicated when calculated stress values remain below the maximum yield value of the materials used, ensuring the bone repair implant can withstand expected physiological loads without failure.

In present embodiments, the adjusting of the model of the bone repair implant includes at least one selected from the group consisting of adjusting a pore volume of the bone scaffold, adjusting a thickness of the support plate, adjusting a length of the support plate, adjusting width profile of the support plate, adjusting a screw placement location, and adjusting a screw number of the bone repair implant. When adjusting the pore volume of the bone scaffold, modifications typically range between 30% to 50% of total scaffold volume. Increasing pore volume generally decreases overall mechanical strength, while decreasing pore volume enhances mechanical strength. The adjustment process seeks to balance mechanical competence with biological requirements, as sufficient porosity is necessary for cell infiltration and vascularization while maintaining adequate structural integrity. When adjusting the thickness of the support plate, modifications typically range between 5 to 7 millimeters. The thickness adjustment directly impacts the plate's ability to withstand bending forces and distribute loads. Increasing plate thickness enhances mechanical strength but may need to be balanced against considerations of patient comfort and tissue irritation. When adjusting the length of the support plate, modifications ensure the plate extends sufficiently beyond both ends of the fracture position to provide adequate fixation surfaces on the proximal bone portion and distal bone portion. The length adjustment ensures proper distribution of mechanical loads across the repair site. When adjusting the width profile of the support plate, modifications ensure the plate maintains proper contact with bone surfaces while providing sufficient area for screw placement. The width profile adjustment optimizes the plate's ability to distribute mechanical loads while maintaining anatomical fit. When adjusting screw placement location, modifications optimize the spacing and positioning of screw holes to distribute mechanical loads evenly across the repair site. The screw holes are typically spaced at defined intervals ranging from 5-10 millimeters apart to ensure optimal fixation while avoiding interference with the healing process. When adjusting screw number, modifications ensure sufficient fixation points while avoiding excessive stress concentration. Multiple screws are typically used, with specific numbers determined based on the length of the support plate and the mechanical requirements of the repair site.

In some embodiments, the bone repair implant further comprises a radiopaque marker. The radiopaque marker enables visualization and monitoring of the bone repair implant position and alignment after surgical implantation using medical imaging techniques. The radiopaque marker provides contrast during imaging procedures such as X-rays, computerized tomography scans, and other radiographic examinations, allowing medical professionals to verify proper placement and assess the healing progress of the bone fracture. The radiopaque marker is particularly important given that the bone repair implant is constructed from biodegradable materials like polylactic acid, polyglycolic acid, or poly lactic acid glycolic acid, which may not be clearly visible under standard medical imaging. By incorporating the radiopaque marker, the method enables post-surgical monitoring of: the position and alignment of the bone scaffold relative to the proximal bone portion and distal bone portion; the proper placement and continued stability of the support plate across the bone fracture site; the position and stability of the screws securing the support plate to the bone portions; the gradual degradation of the bone repair implant components over time; and the progression of new bone formation at the fracture site. The radiopaque marker thus helps in post-operative assessment and long-term monitoring of the bone repair process, allowing healthcare providers to verify successful implant placement and track the healing progression without the need for additional surgical procedures.

In implementations of the method 100, the patient does not require an additional surgery to remove the bone repair implant. This elimination of secondary removal surgery is achieved through the use of biodegradable materials in fabricating the bone repair implant, including the bone scaffold, support plate, and screws. The biodegradable materials undergo controlled degradation that corresponds with the natural bone healing process, which typically requires 10-18 months for completion in cases of segmental bone fractures. The biodegradable nature of the bone repair implant provides several advantages. As the scaffold starts to degrade, bone tissue extends into the scaffold's interior, regulating the regenerative environment and adapting to the maturation of bone tissue. Meanwhile, the mechanical properties of the bone repair implant gradually decrease, and the applied load transitions from the implant to the newly formed bone tissue, avoiding stress shield impact. The degradation creates empty spaces over time that promote enhanced cell infiltration and vascularization throughout the bone repair implant. The degradation profile of the selected biodegradable materials is specifically chosen to match the rate of new bone formation. For example, when polylactic acid is used, it undergoes bulk degradation over a period of 12-16 months, maintaining sufficient mechanical properties during the critical early healing stages while gradually degrading as new bone tissue forms. This timing aligns with the typical bone healing timeline, ensuring adequate support throughout the regeneration process while eliminating the need for surgical removal.

By eliminating the need for additional removal surgery, the method 100 reduces multiple burdens associated with conventional treatment approaches. The elimination of secondary surgical procedures significantly reduces physical trauma to the patient, as no additional tissue disruption or surgical intervention is required after the initial implantation. The recovery time is limited to a single post-surgical period rather than multiple recovery phases that would be necessary with non-biodegradable implants requiring removal. The method 100 provides substantial economic benefits in the treatment of bone fractures. The elimination of secondary removal surgeries through the method 100 reduces these costs by decreasing the number of surgical procedures, shortening the duration of hospitalization, and minimizing associated medical expenses. The reduced number of surgical procedures also decreases the demands on healthcare resources and medical personnel. The method 100 further reduces complications associated with secondary surgical procedures, including the risks of infection, anesthesia-related complications, and potential damage to surrounding tissues during implant removal. The single surgical intervention approach also reduces the cumulative risk of surgical site infections and other procedure-related complications that could arise from multiple surgeries. Further, the gradual degradation of the bone repair implant allows for continuous, uninterrupted bone regeneration. This continuous healing process promotes more efficient tissue regeneration and can lead to improved functional outcomes. Furthermore, the method 100 reduces the financial burden on healthcare systems by decreasing the overall resource utilization associated with treating bone fractures. The reduction in surgical procedures, hospitalization duration, and post-operative care requirements results in more efficient use of healthcare resources and reduced overall treatment costs.

The method 100 of the present disclosure addresses bone fractures, which represent prevalent musculoskeletal conditions requiring medical intervention. In cases of critical-sized segmental bone fractures, where the fracture size exceeds a repairable threshold of 2 centimeters, natural self-healing mechanisms become insufficient, necessitating surgical intervention. The method 100 provides improvements over current clinical treatments including autografts, allografts, and metallic implants, which exhibit limitations in addressing critical-sized segmental bone fractures. The method 100 implements regenerative engineering principles through integration of advanced technologies from material science and engineering fields to enable regeneration of damaged or lost bone tissue. The method 100 combines biodegradable materials with additive manufacturing technology to produce bone repair implants capable of addressing limitations of current treatments. The method 100 enables production of bone repair implants that align with patient-specific anatomy while providing appropriate porosity, biocompatibility, biodegradability, and mechanical properties for enhanced bone repair.

The method 100 includes generating bone repair implants comprising biodegradable bone scaffolds, support plates, and screws customized to patient anatomy. The biodegradable nature of the bone repair implant eliminates requirements for secondary removal surgeries. The method 100 includes evaluation of the bone repair implant through mechanical testing, physical characterization, morphological analysis, and biological testing to verify effectiveness and safety. The method 100 achieves treatment of critical-sized segmental bone defects through implementation of patient-specific biodegradable bone scaffolds, support plates, and screws. The method 100 provides secure fixation and stability while promoting formation and regeneration of fractured bone tissue. The biodegradable materials utilized in the method 100 undergo natural degradation, eliminating secondary surgeries for hardware removal. The method 100 employs additive manufacturing, specifically fused deposition modeling, to fabricate patient-specific bone repair implants using biocompatible and biodegradable materials with controlled mechanical properties. The additive manufacturing process of method 100 enables precise control of scaffold geometry, porosity, and interconnectivity for customizing biodegradable bone repair implants to match specific anatomical requirements of individual patients.

The method 100 includes multiple steps implemented in sequence. Initially, the method 100 employs advanced imaging techniques, specifically computer tomography scans, to acquire detailed anatomical data of patient femoral bones. The method 100 then processes this imaging data through segmentation software and computer-aided design software to generate three-dimensional models. This processing generates precise three-dimensional models of bone defects, enabling recreation of missing bone volume. In particular, these models serve as the basis for designing personalized bone substitutes that closely match the patient's unique anatomy, ensuring an optimal fit and reducing the risk of complications associated with non-customized implants. Subsequently, the method 100 implements additive manufacturing to fabricate porous patient-specific bone repair implants using biodegradable polylactic acid. The method 100 concludes with mechanical testing, biocompatibility evaluation, biodegradation assessment, and microstructural analysis to validate safety and effectiveness of the bone repair implant.

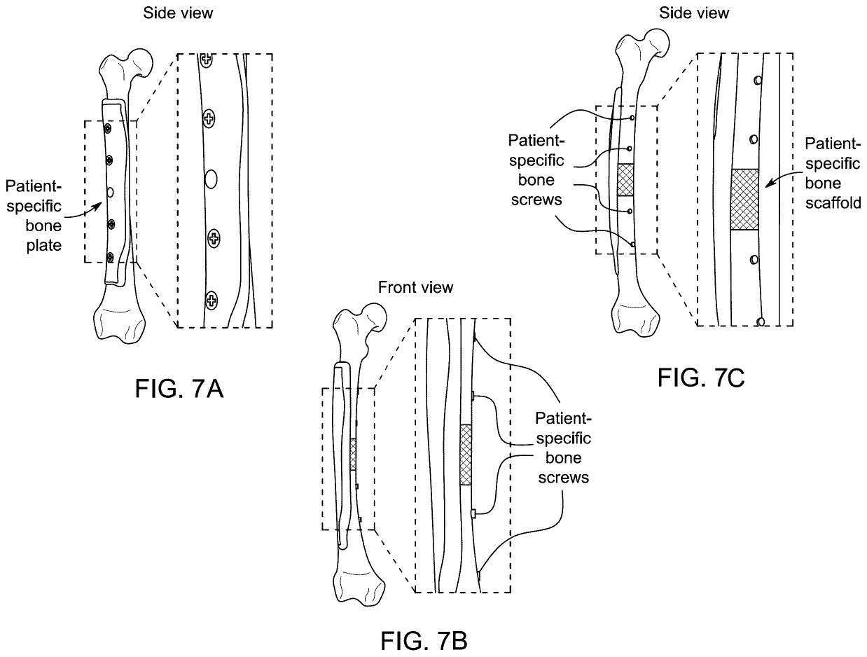

The following paragraphs discuss design and methodology for developing patient-specific and biodegradable 3D-printed bone substitutes, screws, and plates by evaluating the design alternatives and selecting the final design concept. Firstly, detailed anatomical data is obtained through advanced imaging techniques (CT scan). Then, the biodegradable materials used in manufacturing are selected. Finally, the model is designed with several geometrical and dimensional considerations and the results of each are analyzed by conducting tests to verify its efficiency. The proposed work has been broken down into a series of steps. First, advanced imaging techniques, such as computer tomography (CT) scans, are employed to acquire detailed anatomical data from the patient's femoral bones. Segmentation software (3D slicer) and computer-aided design (CAD) software (SolidWorks) are used to convert these CT scans into 3D models, respectively. This process ultimately results in the generation of precise 3D models of the patient's bone defects, which enables to recreate the 3D volume of the lost bone. After that, 3D printing technologies, specifically fused deposition modeling (FDM), are used to fabricate highly porous patient-specific bone substitutes, along with the screws and plates, using the biodegradable and biocompatible polylactic acid (PLA). Lastly, in vitro mechanical testing, biocompatibility evaluations, biodegradation assessments, and microstructural analyses are performed to evaluate and validate the efficacy of the printed bone substitutes, plates, and screws in terms of their safety and strength. The final design of this complete, patient-specific treatment scheme is illustrated in FIGS. 7A-7C.

In bone tissue engineering, biocompatibility and degradation rate are crucial factors to consider when designing bone scaffolds, plates, and screws. Since these components will be introduced into the human body, they may need to be made using a biocompatible material to eliminate immune rejection. Likewise, it is preferable to produce these components using a biodegradable material to eliminate the need for secondary removal surgeries. In this context, various biocompatible and biodegradable materials are available, including poly lactic acid (PLA), poly glycolic acid (PGA), and poly lactic glycolic acid (PLGA) [R. Song, M. Murphy, C. Li, K. Ting, C. Soo, and Z. Zheng, “Current development of biodegradable polymeric materials for biomedical applications,” Drug Des. Devel. Ther., vol. 12, pp. 3117-3145 September 2018, incorporated herein by reference in its entirety]. PLA takes up to 1-1.2 years to completely degrade. On the other hand, PGA and PLGA exhibit a faster degradation rate and completely degrade within 4 weeks and 4 months, respectively. Given the prolonged healing time of bone, which takes up to 1-1.2 years to completely heal and regenerate after critical-sized segmental bone fractures, PLA stands out as a more suitable choice for current application. Consequently, it was decided to choose PLA as the material for the fabrication of the bone scaffolds, plates, and screws.

In addition to biocompatibility and biodegradation, the mechanical competence and strength achieved in these components is an important factor to consider. This is due to the fact that these components will be placed in load-bearing sites within the bone structure. They will be continuously exposed to mechanical loads, making it crucial for them to withstand these stresses to prevent implant failure after transplantation into the human body. In this context, various design alternatives were explored for each component, including scaffold pore volume, screw diameter, and plate thickness. The alternatives for each component are thoroughly discussed below.

Scaffold is the main component in this entire treatment scheme as it will fill the bone defect site, acting as a temporary structure to promote bone formation. The bone scaffolds may need to display high mechanical strength to provide adequate support during the healing process. Additionally, introducing a high pore volume into the scaffold's structure is essential to enable enhanced cell infiltration, vascularization, and bone ingrowth and regeneration. However, it is important to note that while increasing pore volume is beneficial for tissue regeneration, it may impact the mechanical properties of the scaffold. Therefore, a careful balance between mechanical strength and porosity may need to be investigated and determined during the design process to ensure optimal scaffold performance. In this sense, various pore volume percentages were considered including, 50%, 40% and 30%. After careful considerations and literature search, it was decided to select 40% pore volume. This selection arises from the fact that, while bone scaffolds with 50% porosity may display enhanced cellular infiltration, vascularization, and bone regeneration; they may not be able to withstand the harsh mechanical loads being applied in the bony environment. Likewise, bone scaffolds with 30% porosity may indeed display adequate mechanical resilience, but may not promote sufficient cellular infiltration, vascularization, and bone regeneration as shown in previous research. Therefore, it is believed that a bone scaffold with 40% porosity may achieve a balance between mechanical strength and facilitating enhanced bone growth. Still, further computational testing and in vitro validation will be required to confirm this, as discussed in the proceeding paragraphs.

Bone plates are medical devices used during surgical procedures to stabilize fractured bones after an injury. When a bone is fractured, bone plates are affixed to the surface of the bone using screws. This helps in preserving the alignment and functionality of the bone, allowing for optimal healing. Therefore, it is extremely important that bone plates exhibit excellent mechanical competence, as the effectiveness of bone fixation and stability relies significantly on their strength. The currently available bone plates are mainly made from metals such as stainless steel or titanium, due to their biocompatibility and exceptional strength [F. D. Al-Shalawi et al., “Biomaterials as Implants in the Orthopedic Field for Regenerative Medicine: Metal versus Synthetic Polymers,” Polymers, vol. 15, no. 12, Art. no. 12, January 2023, incorporated herein by reference in its entirety]. However, the need for a secondary removal surgery after bone healing is complete is the main limitation of these metallic bone plates. Therefore, one of the aims of the present disclosure is to fabricate these bone plates using an entirely biodegradable material, specifically PLA, to ensure their absorption overtime thereby, eliminating the need for additional removal surgeries. Current metallic bone plates typically have thicknesses ranging between 2-2.5 mm. However, since the present implementations involves fabricating these plates using PLA, which is mechanically weaker, it is important to consider increasing the overall thickness of the biodegradable plate beyond the standard thicknesses currently used for metallic plates to ensure maintaining the plate's mechanical resilience. Therefore, various thicknesses were considered including, 5 mm, 6 mm, and 7 mm. After careful considerations, it was decided to fabricate the biodegradable bone plates with a final thickness of 7 mm to ensure the best possible mechanical competence. Thicknesses beyond 7 mm were not considered as these bone plates are meant to be placed on the surface of the bone inside of the body, and thicker plates than 7 mm may not be convenient to the patient. While it is believed that a PLA-based bone plate with an overall thickness of 7 mm may provide sufficient strength and fixation, further computational testing and in vitro validation is required to confirm this, as discussed later.

Bone screws are medical devices used during surgical procedures to secure or fixate bones, plates, or implants to the skeleton structure. Therefore, it is extremely important that bone screws exhibit excellent mechanical competence to withstand mechanical loads. Bone screws are typically made of biocompatible and strong materials such as stainless steel and titanium. However, like metallic bone plates, these metallic bone screws necessitate secondary removal surgeries after bone healing is achieved. Therefore, one of the aims of the present disclosure is to fabricate these bone screws using an entirely biodegradable material, such as PLA, to ensure their absorption overtime thereby, eliminating the need for additional removal surgeries. Current metallic bone screws typically have diameters ranging between 4-8 mm. However, since the proposed implementations involve fabricating these screws using PLA, which is mechanically weaker, it is important to consider fabricating these PLA-based screws with the largest possible diameter to ensure maintaining the screw's mechanical resilience. Therefore, various diameters were considered including, 6 mm, 7 mm, and 8 mm. After careful considerations, it was decided to fabricate the biodegradable bone screws with a final diameter of 8 mm to ensure the best possible mechanical competence. Diameters beyond 8 mm were not considered as screws with a diameter larger than 8 mm may cause soft tissue irritation, bone damage, limited bone healing, and compromised fixation stability. While it is believed that a PLA-based bone screw with an overall diameter of 8 mm may provide sufficient strength and fixation, further computational testing and in vitro validation is required to confirm this, as discussed later.

As discussed, the aim of the present disclosure is to design and develop patient-specific and biodegradable 3D-printed bone substitutes, screws, and plates for critical-sized segmental bone defects. In order to achieve this, CT scan images of patients' femoral bone is required. Herein, the design methodology used for designing the patient-specific bone scaffold, plate, and screws is included.

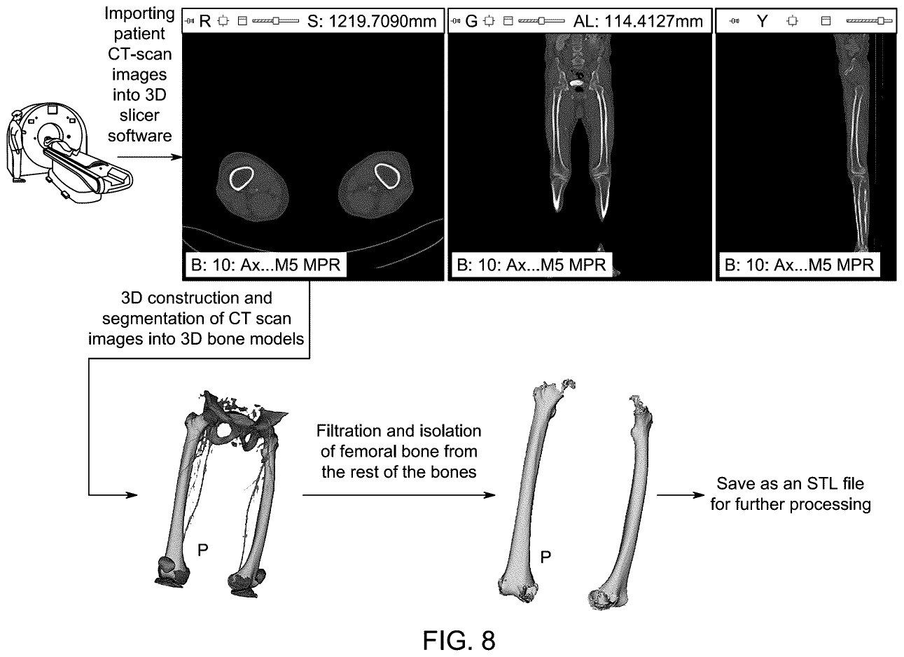

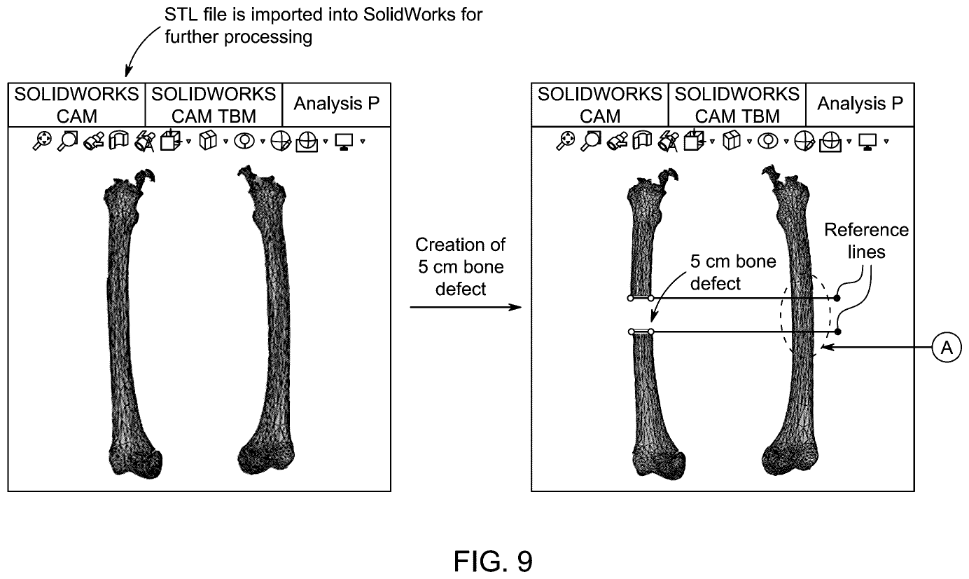

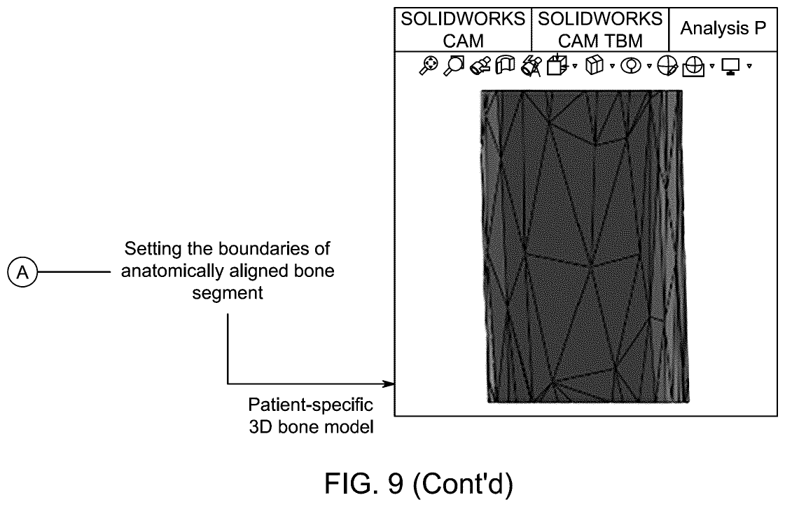

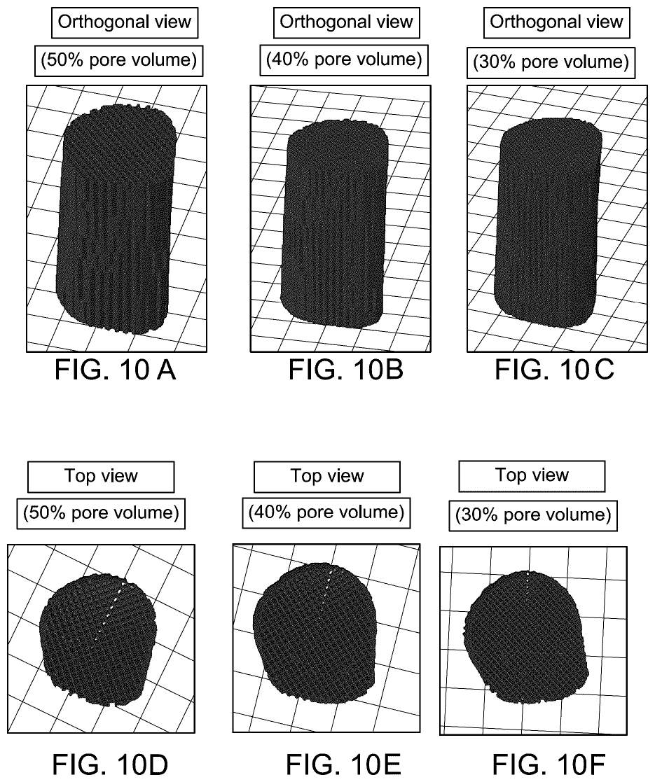

For the purpose of the present disclosure, CT scan images of patient's (54 years old, male patient) femoral bones were obtained from Imam Abdulrahman bin Faisal University Hospital after acquiring the patient's consent and proper documentations. These CT scans can then be imported into a segmentation software (3D Slicer, version 5.5.0), where they are converted into 3D models and subsequently exported as STL files (as depicted in FIG. 8). The 3D femoral bone STL models can then be imported into a computer aided design (CAD) software (SolidWorks) for further processing (as depicted in FIG. 9). Within SolidWorks, precise measurements were taken. Initially, two horizontal lines were drawn from the proximal and distal edges of the defect (assuming a defect size of 5 cm) and extended horizontally to the contralateral uninjured bone (as shown in FIG. 9). These lines serve as a baseline for recreating the bone defect in the contralateral uninjured femoral bone. By drawing these lines and extending them horizontally to the contralateral uninjured bone, it is possible to identify the segment that aligns anatomically with the defect in the injured bone (as shown in FIG. 9). This segment can then be mirrored to match the anatomy of the defect site in the injured bone, resulting in the final 3D model. This model serves as the basis for creating the patient-specific bone scaffold. Next, pore volumes of 30%, 40%, and 50% were introduced in the scaffold's design within the SolidWorks software (as depicted in FIGS. 10A-10F).

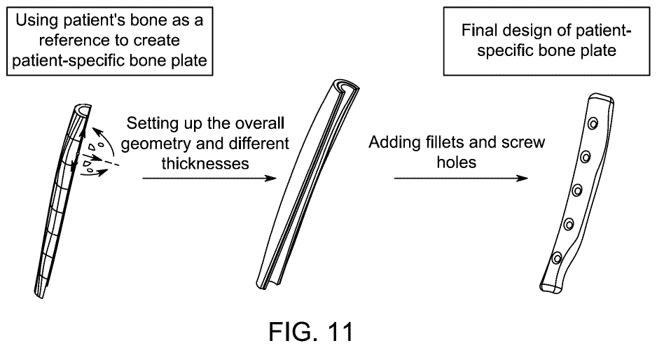

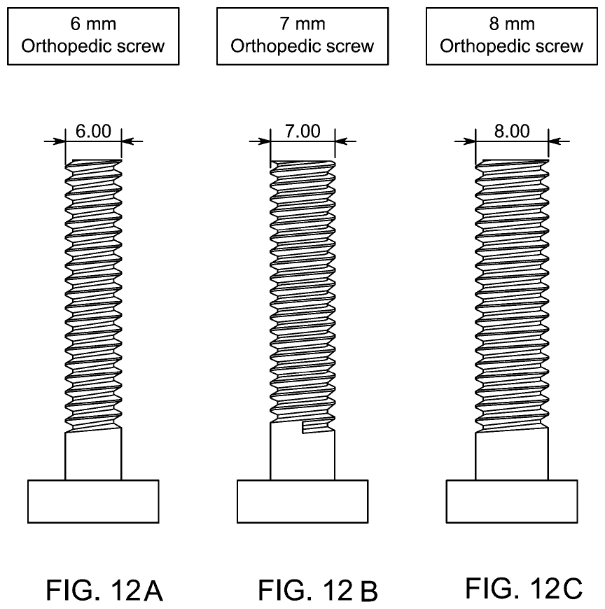

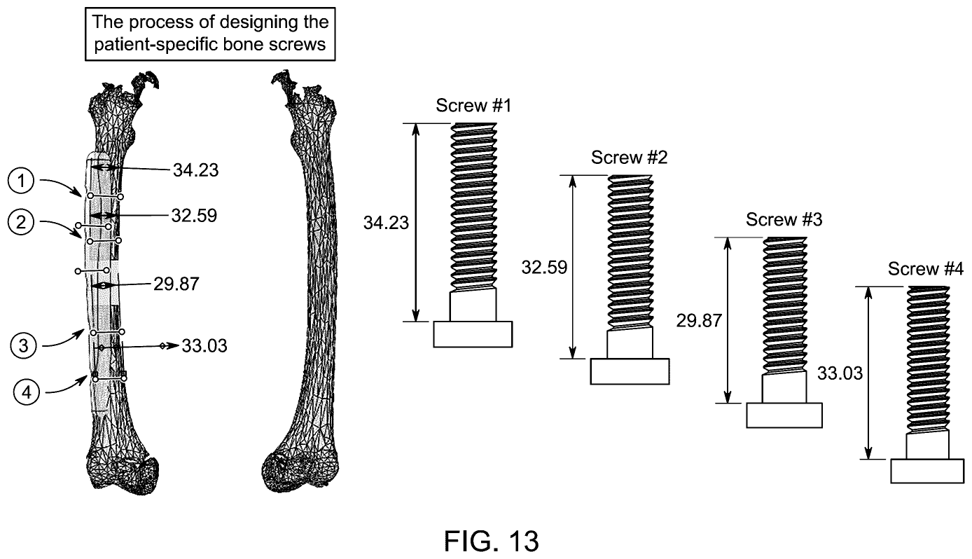

To design the patient-specific bone plates, the patient's bone can be used as a reference to create a bone plate that matches the patient's own anatomy (as depicted in FIG. 11). These bone plates morphologically matches the patient's geometry, whereas they are produced with verifying thicknesses of 5, 6, and 7 mm. To design the bone screws, the process can be started with a standard cortical bone screw design and make specific modifications. In summary, these adjustments will result in screws with final diameters of 6, 7, and 8 mm (as depicted in FIGS. 12A-12C). Additionally, the screws will be patient-specific, with their length adjusted to match the diameter of the patient's bone for proper fixation and patient convenience (as depicted in FIG. 13).

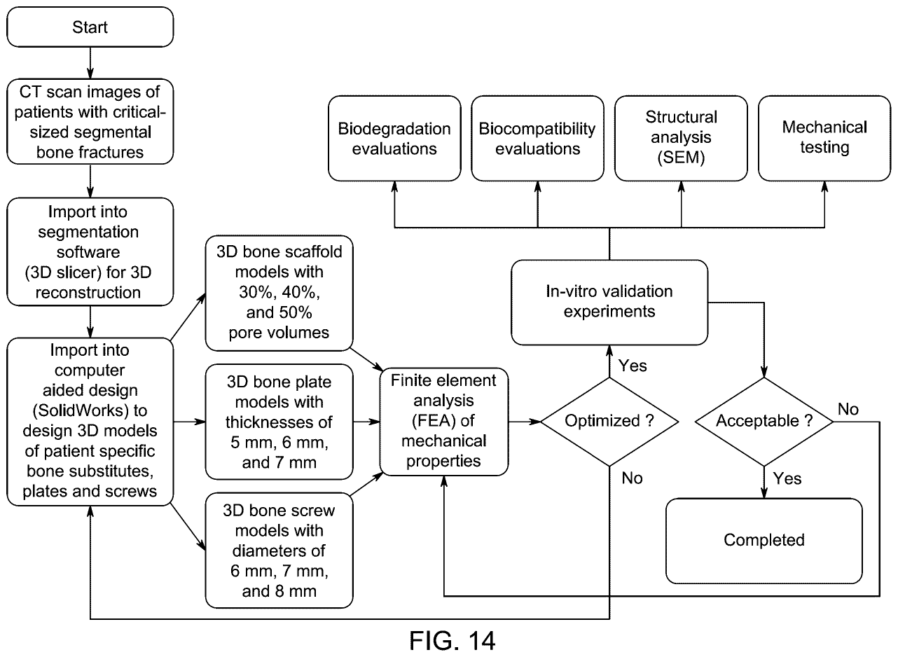

Further, in implementation of the present disclosure, the designed bone scaffolds, plates, and screws are subjected to numerical verification and subsequent in vitro validation to ensure safety and efficacy. FIG. 14 illustrates the design process flow chart for the scaffolds, plates, and screws.

Next, finite element analysis (FEA) of the mechanical properties of the bone scaffolds, plates, and screws are performed and numerical values are generated. To perform the FEA, SolidWorks software was used. SolidWorks is a computer-aided design (CAD) software developed by Dassault Systèmes. It is widely used in engineering and design for creating 3D models, simulations, and technical documentation. SolidWorks offers a range of tools and features that makes it a versatile and powerful solution for various design and engineering applications. One of the tools that SolidWorks offers is FEA of the mechanical properties of 3D models, as discussed below.

The first step involves applying the desired material to the 3D model from the library of materials found in the software. Given that the 3D models will ultimately be fabricated using PLA, the software was configured with PLA as the designated material for precise analysis. Next, the boundaries are defined, including the constraint surfaces and the areas where physiologically relevant compression forces would be applied to the 3D models. After defining the boundaries, a specific force value, in (newton, N) is applied, and the problem is solved to estimate the mechanical strength of the 3D models.

The FEA was performed on the three different components, the scaffolds, screws, and plates. For each component, FEA was conducted on all alternatives (as discussed later in the description). Herein, the analysis and results of the FEA performed is presented and analyzed.

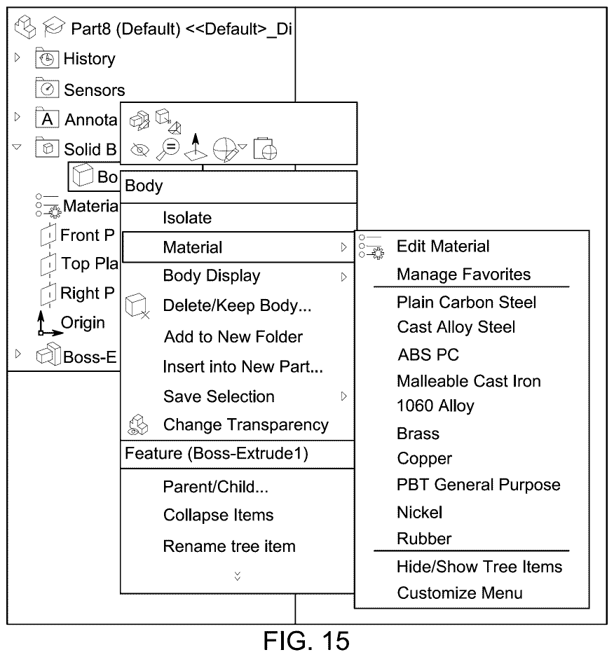

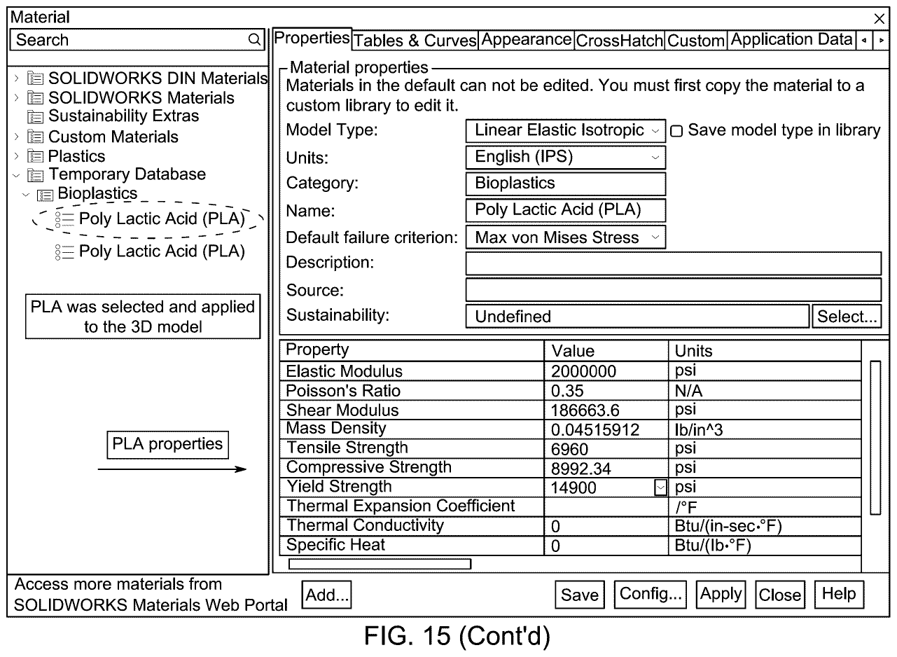

The mechanical test assesses the stress of a part or assembly when subjected to a specific load in compression or torsional modes. This evaluation is crucial for analyzing structural integrity, identifying regions of high stress, and predicting potential failure points. To perform the mechanical analysis, PLA was first set as the material for each component (as depicted in FIG. 15). Subsequently, the boundaries were defined, including the constraint surfaces and the areas where physiologically relevant forces would be applied to the 3D models (as depicted in FIG. 16). The force value was set to 800 N, which is equivalent to a body weight of 80 kg. This weight was selected as it represents the average weight of humans [S. C. Walpole, D. Prieto-Merino, P. Edwards, J. Cleland, G. Stevens, and I. Roberts, “The weight of nations: an estimation of adult human biomass,” BMC Public Health, vol. 12, no. 1, p. 439, June 2012, incorporated herein by reference in its entirety]. Next, the analysis was run to solve the problem. The stress and deformation data for each component's alternative [scaffolds (with pore volumes of 50%, 40%, and 30%), plates (with thicknesses of 5 mm, 6 mm, and 7 mm), and screws (with diameters of 6 mm, 7 mm, and 8 mm)] were exported. To mimic the in vivo loading conditions, the scaffolds underwent vertical compression testing, whereas the screws and plate underwent a torsional (torque) and a horizontal 4-points compression testing, respectively.

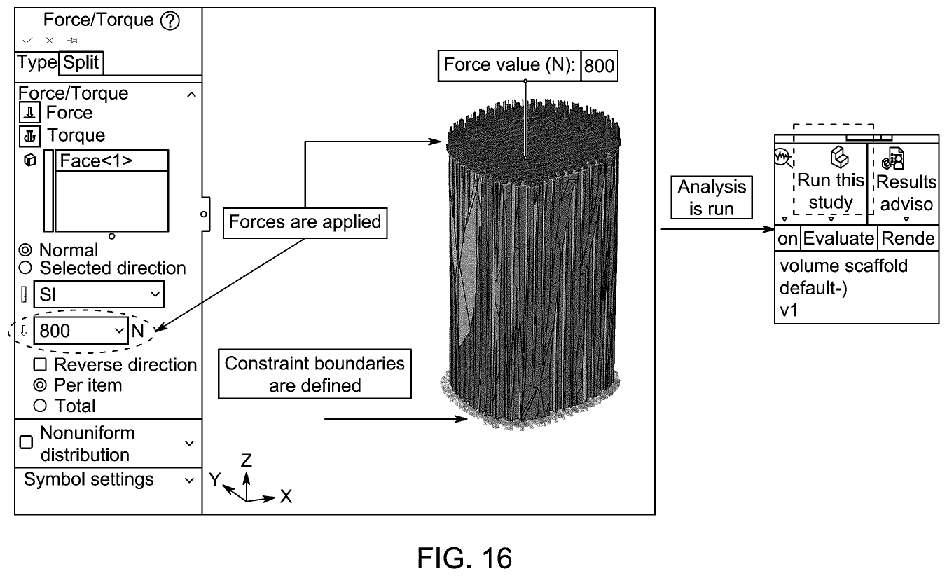

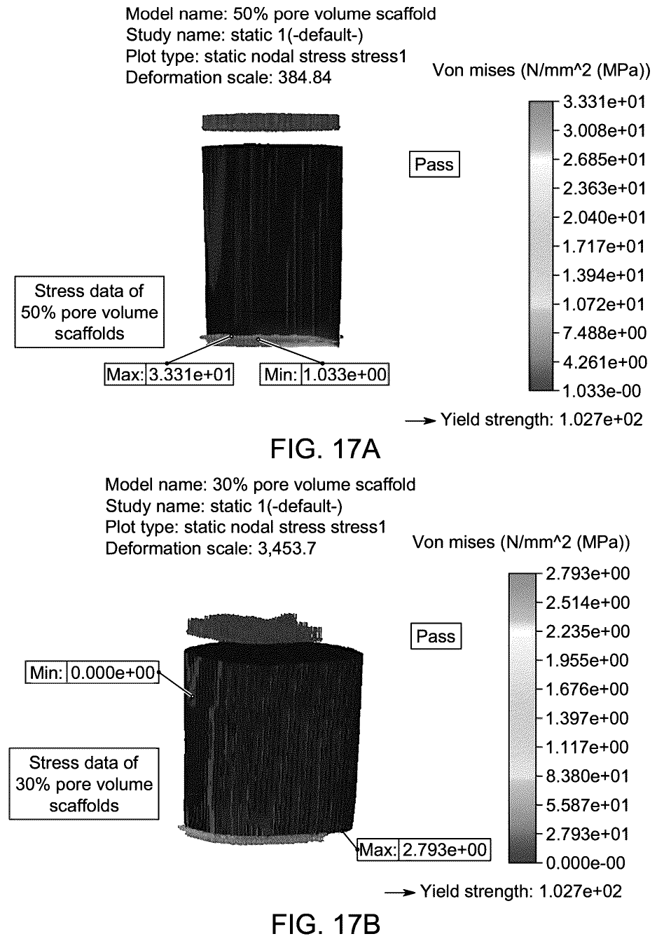

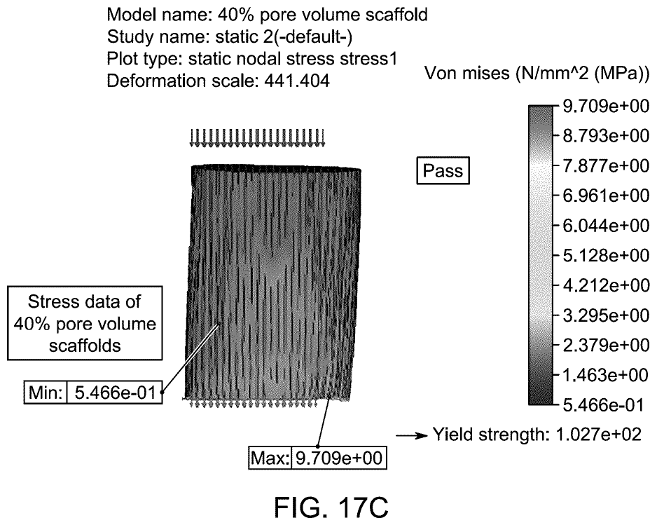

FIGS. 17A-17C shows the strain data for the different scaffolds. As expected, increasing the pore volume in the scaffold from 30% to 50% resulted in decreasing the scaffold's overall mechanical strength. Specifically, the stress values for the scaffolds with pore volumes of 30%, 40%, and 50% was found to be 2.79 MPa, 9.7 MPa, and 33.31 MPa, respectively. The observed increase in stress values with increasing the pore volume suggests a decrease in the scaffold's overall mechanical strength. It may be noted that higher stress values correspond to weaker scaffolds. Nevertheless, all scaffolds demonstrated the ability to withstand the applied load of 800 N without failure, indicating their suability for the intended load-bearing application. This is evident from the fact that all scaffolds exhibited stress values lower than their maximum yield value of 127 MPa (maximum stress value they can withstand before failing).

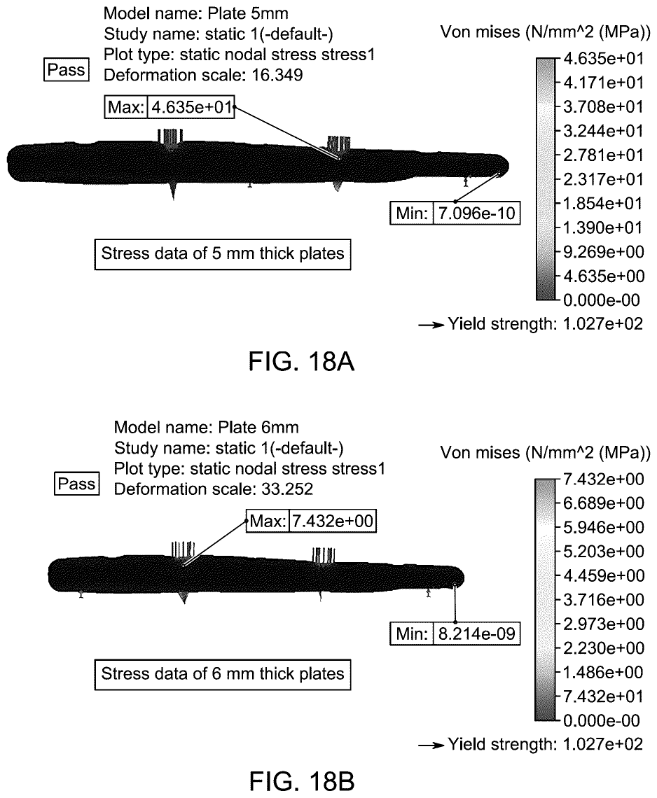

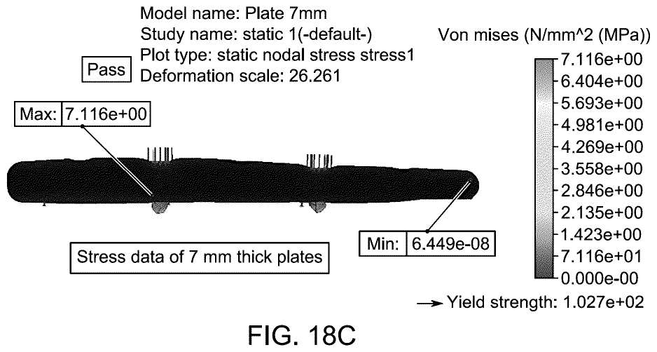

FIGS. 18A-18C shows the strain data for the different plates. As anticipated, increasing the thickness of the plate from 5 mm to 7 mm resulted in increasing the plate's overall mechanical strength. Specifically, the stress values for the plates with thicknesses of 5 mm, 6 mm, and 7 mm was found to be 46.35 MPa, 7.43 MPa, and 7.11 MPa, respectively. The observed increase in stress values with decreasing the plate's thickness suggests a decrease in the plate's overall mechanical strength. It may be noted that higher stress values correspond to weaker plates. Nevertheless, all plates demonstrated the ability to withstand the applied load of 800 N without failure, indicating their suability for the intended load-bearing application. This is evident from the fact that all plates exhibited stress values lower than their maximum yield value of 127 MPa (maximum stress value they can withstand before failing).

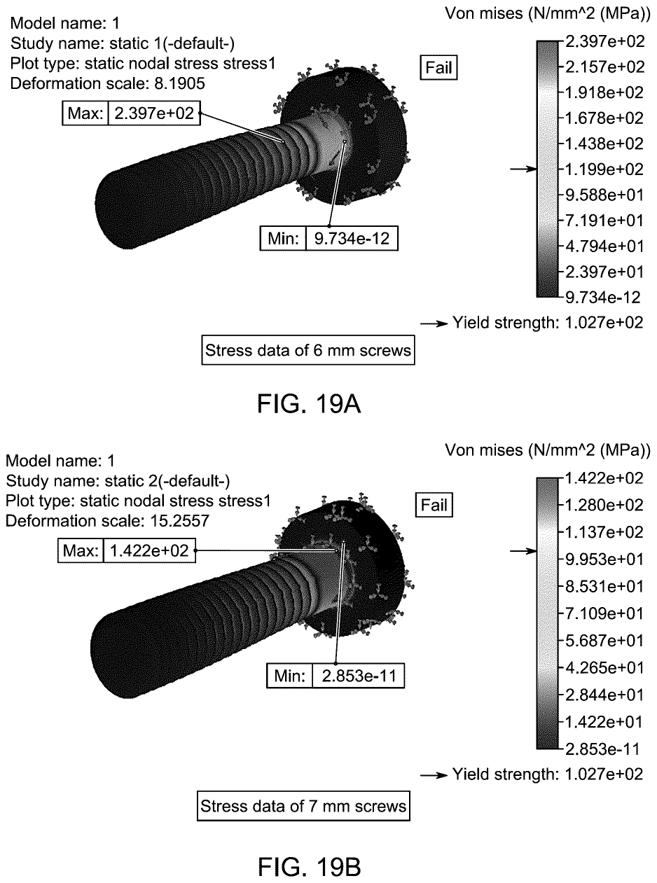

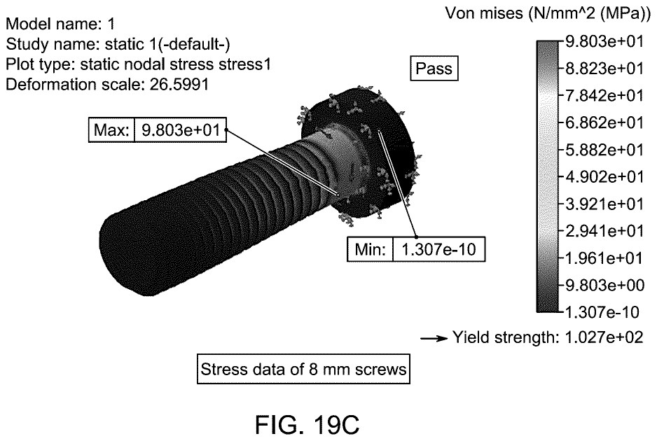

FIGS. 19A-19C shows the strain data for the different screws. As predicted, increasing the screw's diameter from 6 mm to 8 mm resulted in increasing the screw's overall mechanical strength. Specifically, the stress values for the screws with diameters of 6 mm, 7 mm, and 8 mm was found to be 239 MPa, 142 MPa, and 98.03 MPa, respectively. This indicates that as the screw's overall diameter was increased, its mechanical strength increased. However, the data revealed that only screw with a thickness of 8 mm could withstand the applied load without failure, whereas the screws having diameters of 6 mm and 7 mm experienced complete failure. This is evident by the fact the screw with a thickness of 8 mm had a stress value of 98.03 MPa, which is lower than its yield strength value of 127 MPa. On the other hand, the stress values for the screws with thicknesses of 6 mm and 7 mm were 239 MPa and 142.2 MPa, respectively. These values exceeded the screw's yield strength, indicating failure.

Further, since these components are intended to be utilized in load-bearing anatomical sites, they may need to be designed with robust mechanical properties. Consequently, the scaffolds, plates, and screws were initially designed with varying pore volumes, thicknesses, and diameters, respectively. Computational analysis of their mechanical competence and robustness was then conducted to finalized the optimal design for each component. Based on the FEA conducted above, it was found that all scaffolds demonstrated the ability to withstand the applied mechanical load without failure, indicating their suitability. However, considering previous literature suggesting that scaffolds with a pore volume of 30% may not promote sufficient cellular infiltration, vascularization, and bone regeneration [E. K. Cushnie et al., “Simple Signaling Molecules for Inductive Bone Regenerative Engineering,” PLOS ONE, vol. 9, no. 7, p. e101627, incorporated herein by reference in its entirety], and recognizing the potential failure in vivo with scaffolds exhibiting 50% pore volume, it was decided that the final scaffold design will exhibit a 40% pore volume. This decision aims to achieve a balance by maintaining mechanical strength while offering an adequate pore volume for the intended purposes. Further FEA revealed that a bone plate and screw with a final thickness of 7 mm and a diameter of 8 mm, respectively, exhibited the best resistance to the applied mechanical load without failure, confirming their suitability. Therefore, it was concluded that the final design for the plates and screws would feature a thickness of 7 mm and a diameter of 8 mm, respectively.

As discussed, all components including the scaffolds, plates, and screws are fabricated using PLA. This material was chosen since it is biocompatible, biodegradable, and can easily be 3D printed. Table 2 (below) outlines the cost of materials utilized in the design and fabrication of scaffolds, screws, and plates as per embodiments of the present disclosure.

TABLE 2
A list of the Materials utilized in fabrication of
scaffolds, screws, and plates and associated cost.

| Item Name | Amount/volume/QTY | Total Price (SAR) |
|---|---|---|
| CT scans | NA | Provided by university |
| | | hospital |
| SolidWorks software | NA | Provided by IAU |
| 3D Slicer software | NA | Free |
| 3D slicing software | NA | Free |
| FDM 3D printer | NA | Provided by IAU |
| PLA filament | 2 kg | 200 |

*NA indicated (Not Applicable)

As shown in Table 2, it is evident that the proposed approach is cost-effective, as the only expense to be considered is the cost of the polymer. For more accurate cost analysis, the weight of the scaffold, plate, and screws intended for a single patient was measured, which allowed to precisely estimate the total cost for all components based on their weight. Furthermore, while the PLA (as used) is biocompatible and biodegradable, it is not medical-grade, and therefore, it is cost-effective. Medical grade PLA costs around 26.25 SAR per gram. In cost analysis, the price of the medical grade PLA was considered for a more accurate reflection of real-world costs. Yet, a cost analysis using the PLA (as used) was also included for documentation purposes. Table 3 (below) outlines the weight of each component and its corresponding cost:

TABLE 3
Cost analysis of proposed technology per patient.

| | | Price/ gram | Price/ gram | | Total price | Total price |
|---|---|---|---|---|---|---|
| | Weight | (Medical | (non-medical | QTY | (medical | (non-medical |
| Component | (g) | Grade) (SAR) | Grade) (SAR) | needed | grade) (SAR) | grade) (SAR) |
| Scaffold | 19 | 26.25 | 0.1 | 1 | 498.75 | 1.9 |
| Plate | 204 | 26.25 | 0.1 | 1 | 5355 | 20.4 |
| Screw | 2.53 | 26.25 | 0.1 | 5 | 332.06 | 1.26 |
| | | | | Total net | 6185.81 | 23.56 |
| | | | | price | | |

As shown in Table 3, the total cost for implementing the proposed technology, which includes a complete set of biodegradable scaffolds, plates, and screws, is estimated to cost 6185.81 SAR and 23.56 SAR for the medical and non-medical grade PLA, respectively, per 10 patient. Compared to current technologies, including allografts, autografts, and metallic implants, and the associated metallic plates and screws often used with them, the proposed technology represents a more cost-effective alternative, as further demonstrated in Table 4 (below).

TABLE 4
Cost analysis of current technologies per patient.

| Component | Price (SAR) | QTY needed | Total price (SAR) |
|---|---|---|---|
| Autograft | 9,375-13125 | Patient-specific | 9,375-13125 (Price |
| | | | of harvesting) |
| Allograft | 3312.49 | Patient-specific | 3312.49 |
| Metallic | 6088.50 | Patient-specific | 6,088.50 |
| implant | | | |
| Metallic | 2500-5000 | 1 | 2500-5000 |
| plate | | | |
| Metallic | 500-1500 | 5 | 2500-7500 |
| screw | | | |
| | | Total net price | 14,375-25,625 |
| | | for autograft- | |
| | | based treatment | |
| | | Total net price | 19481.9625 |
| | | for allograft- | |
| | | based treatment | |
| | | Total net price | 11,088.5-18,588.5 |
| | | for metallic | |
| | | implant-based | |
| | | treatment | |

It may be emphasized that the cost of hospitalization, procedure, and rehabilitation were excluded in the cost analysis, since the focus here is to estimate the cost of the developed technology compared to other currently available alternatives. However, it is assumed that the overall cost for these elements would decrease if the proposed technology was to be implemented. This reduction is attributed to the fact that secondary removal surgeries are eliminated, given that biodegradable materials are being used that will degrade overtime. Thus, any costs associated with hospitalization, procedure, and rehabilitation for the second removal surgeries are effectively eliminated.

Furthermore, a series of in vitro experiments were conducted to assess and confirm the accuracy, safety, and efficacy of the proposed system, including microstructural scanning electron microscopy (SEM) analysis, mechanical testing, biodegradation assessments, and biocompatibility evaluations. A detailed description of the design of the experimental setup is presented in the following paragraphs. In addition, the below description presents the results obtained from the conducted experiments, highlighting the data collected and a comprehensive analysis of the findings.

To design the patient-specific bone scaffolds, computer tomography (CT) scans of patients' femoral bones were obtained from Imam Abdulrahman bin Faisal University Hospital after acquiring the patients' consent and proper documentations. These CT scans were imported into a segmentation software (3D Slicer, version 5.5.0), where they were converted into 3D models and subsequently exported as STL files. The 3D femoral bone STL models were then imported into a computer aided design (CAD) software (SolidWorks) for further processing. Within SolidWorks, precise measurements were taken. Initially, the distance from the hip joint down to the proximal edge of the defect was recorded. Subsequently, the distance from the proximal edge of the defect to the distal edge was also measured and documented. These measurements served as a baseline for recreating the bone defect in the contralateral uninjured femoral bone. By applying the same measurements to the uninjured contralateral femoral bone, the segment that aligns anatomically with the defect in the injured bone were identified. This segment was mirrored to match the anatomy of the defect site in the injured bone, resulting in the final 3D model. This model served as the basis for creating the patient-specific bone scaffold. Next, the generated model was imported into a slicing software (PrusaSlicer version 2.7.1) for further refinement. Within the PrusaSlicer, infill percentages of 50%, 60%, and 70% were implemented, yielding 3D bone scaffolds with corresponding porosities of 50%, 40%, and 30%.

To design patient-specific bone plates, the 3D model of the patient's femoral bone was imported into SolidWorks software. The Power Surfacing feature was employed to design a bone plate tailored to the specific anatomy of the lateral part of the patient's femoral bone, yielding a patient-specific bone plate. Subsequently, the same bone plate was replicated with varying final thicknesses of 5, 6, and 7 mm.

To design patient-specific bone screws, the diameter of the patient's femoral bone 3D model was initially measured at four separate locations (two distally and two proximally). These diameters were then utilized to determine the length of the screws, ensuring that they are tailored to the patient's specific bone dimensions for enhanced convenience. Subsequently, the same screws were replicated with different final diameters of 6, 7, and 8 mm.

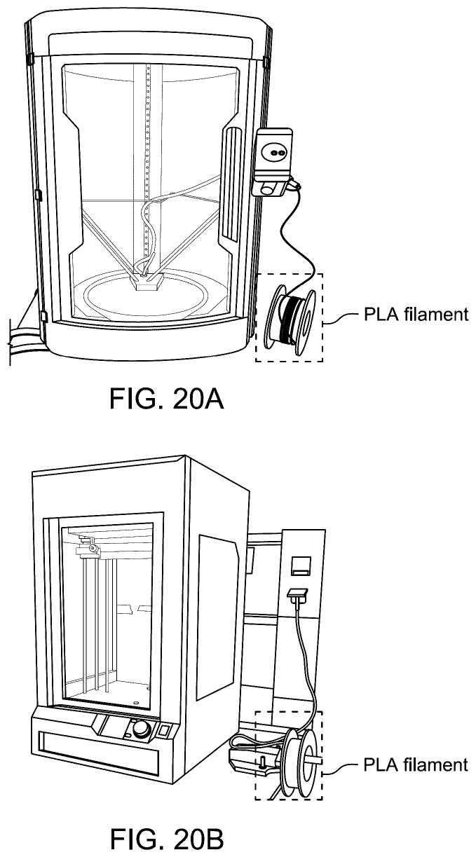

The patient-specific bone scaffolds, along with the screws and plates, were manufactured using biodegradable poly lactic acid (PLA) through 3D printing technology, using a fused deposition modeling (FDM) technique. A 3D printer (Prusa TRILAB AzteQ Industrial) was used to fabricate the scaffolds (as depicted in FIG. 20A), while the (MakerBot Replicator Z18) was used to fabricate the screws and plates (as depicted in FIG. 20B).

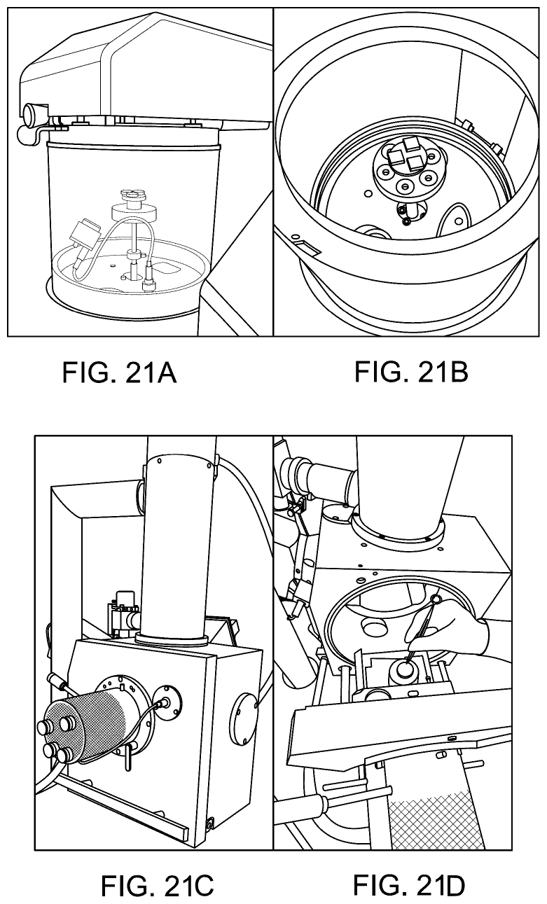

In an implementation, for in vitro experiments, Scanning Electron Microscopy (SEM) was employed. The goal of this procedure was to visualize the internal microstructure of the fabricated scaffolds. This assessment may help determine the printing accuracy (pore size) and evaluate the interconnectivity within the scaffolds' internal structure. To assess the microstructure of the scaffolds, they were mounted on 15 mm stubs, gold sputter-coated using a sputter cotter machine for 3 minutes to eliminate surface charging and imaged using an SEM machine at a working distance of 5 mm and an acceleration voltage of 18 kV (as depicted in FIGS. 21A-21D). The SEM images were used to measure the pore size in each scaffold group using the ImageJ software (version 1.4 g, National Institute of Health, USA).

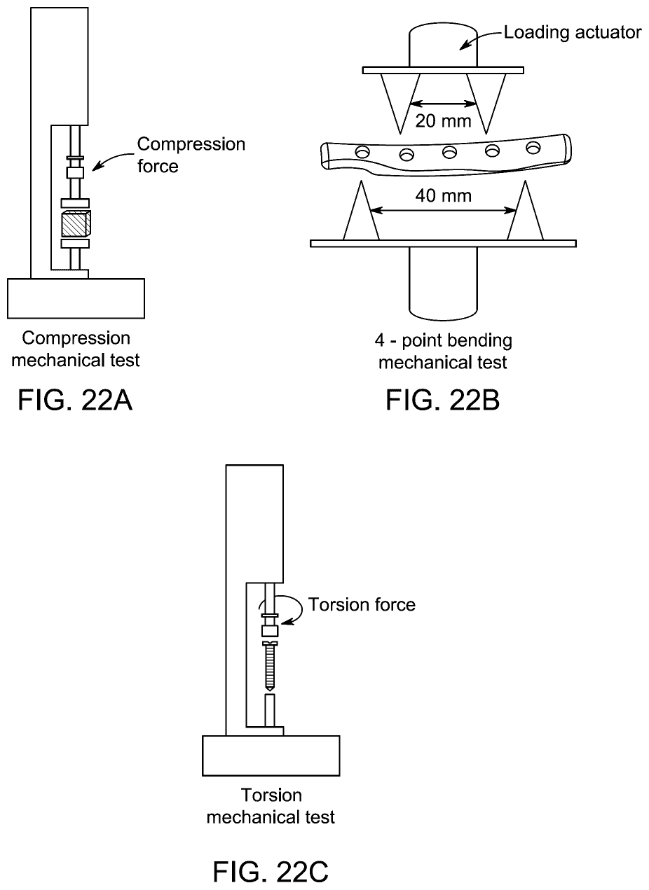

Further, mechanical testing was employed. The goal of this procedure was to assess the mechanical integrity of the scaffolds, screws, and plates in their capacity to withstand compression and torsion loads. This evaluation was crucial, as they may be placed in a load-bearing anatomical site. To assess the mechanical strength of the scaffolds, they were placed perpendicularly between two metallic plates of a mechanical tester machine (Instron) and compression load was applied onto the scaffold until a complete scaffold failure is achieved (N=6/group). The generated force (in newtons) and displacement (in mm) data were used to calculate the compressive strength and compressive modulus. To assess the mechanical strength of the bone plates, a 4-point bending mechanical test was conducted. Briefly, the bone plates were placed horizontally onto two supporting beams spaced at a distance of 40 mm apart within the Instron mechanical tester machine (N=6/group). Subsequently, two loading piece spaced at a distance of 20 mm was descend towards the sample until contact was made, and the test proceeded until failure occurred. The generated force (in newtons) and displacement (in mm) data was used to calculate the compressive strength and compressive modulus. To assess the mechanical strength of the bone screws, a torsion test was performed using a mechanical tester (Instron machine) (N=6/group). Briefly, the screws were positioned between two grips (one fixed and one rotating grip), and each end of the screws was attached to the grips. Next, a torsion force was applied from one end until a complete failure was achieved. The threshold torque (TT), peak failure torque (PFT), and peak clamping torque (PCT) values were measured and recorded for each sample. FIGS. 22A-22C illustrate the setup of the mechanical tests that were performed on the fabricated bone scaffolds, plates, and screws.

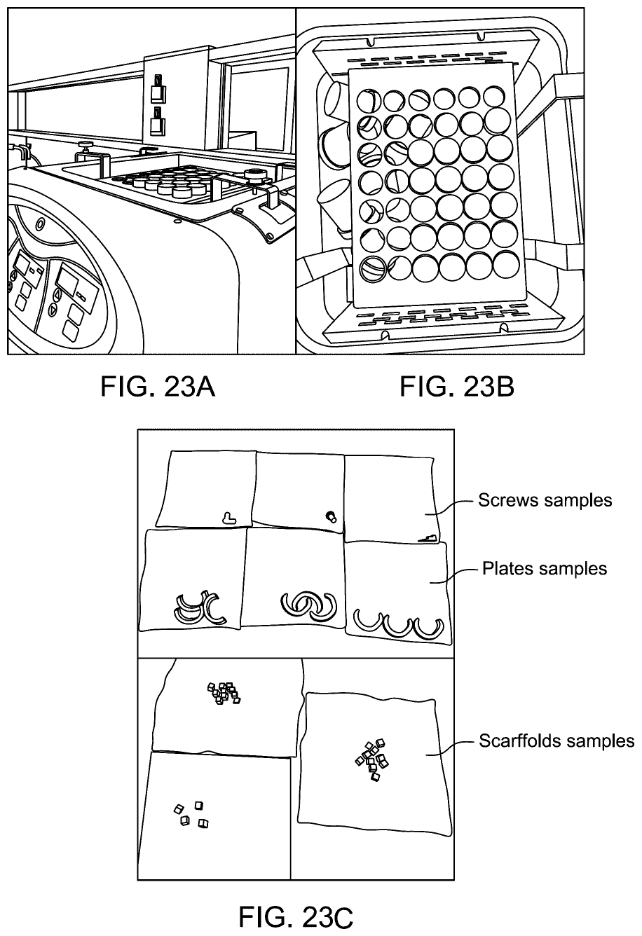

Further, biodegradation testing was employed. The objective of this procedure was to evaluate the biodegradation rate of the implants. This assessment may guide in designing implants with structures that biodegrade in proportion to the rate of new bone formation. To assess the biodegradation of the bone scaffolds, plates, and screws, their initial weights (Wi) was recorded. The samples were then transferred into 50-mL tubes containing 50 mL of water and maintained at 37° C. under gentle agitation (as depicted in FIGS. 23A-23C). The water was changed every one week. After two months, the samples were harvested, frozen, freeze dried, then weighed (Wf). The biodegradation of the samples (N=3/group) was calculated using the following formula:

$$Biodegradation \% = \frac{W i - W f}{W i} \times 100 \qquad ( 1 )$$

where (Wi) is the initial weight, and (Wf) is the final weight.

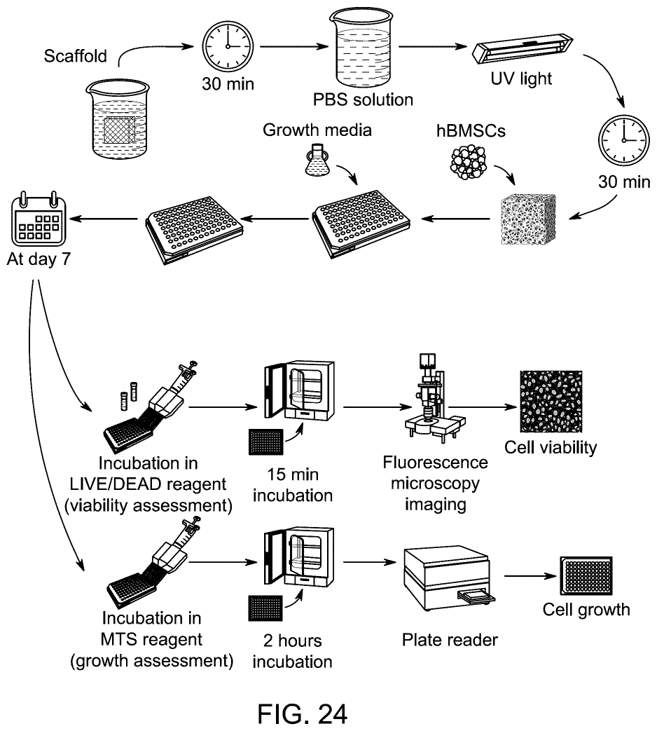

Further, biocompatibility testing was employed. Human Bone Marrow-derived stem cells (BMSCs) were used to assess the biocompatibility of the resultant scaffolds. Briefly, BMSCs were cultured in growth media (DMEM: F12, Gibco, USA) supplemented with 1% penicillin and streptomycin (P/S, Gibco) and 10% fetal bovine serum (FBS, Gibco) in T-182 flasks and maintained in a humidified tissue culture incubator at 37° C. and 5% CO₂. Cells at the 4^{th}-6^{th} passage were used for all experiments. Media was changed every 2 days for all experiments. The objective of this procedure was to assess the biocompatibility of the resulting scaffolds, evaluating their capability to support cell viability and growth within their structures. To assess the biocompatibility of the scaffolds, they were first sterilized. For sterilization, the scaffolds were submerged in 70% ethanol for 30 minutes, washed twice with PBS, then exposed to ultra-violet (UV) light for 30 minutes each side. Next, BMSCs were seeded onto 5 mm×5 mm scaffolds at a final density of 100k cells/scaffold. The seeded scaffolds were then transferred into 96-well plates, and 200 μL of growth media was added to each well. Cell viability was assessed using LIVE/DEAD cytotoxicity assay kit (Invitrogen, USA) at day 7 of culture. Briefly, at day 7, the scaffolds were transferred to new wells, rinsed twice with phosphate buffered saline (PBS Gibco), and then incubated with 100 μL of the kit is staining solution (100 μL PBS, 0.05 μL calcein AM, and 0.2 μL ethidium homodimer-1) for 15 minutes at room temperature. Afterward, the samples were washed twice with PBS and imaged in PBS using an inverted fluorescence microscope. Cell growth was assessed and quantified (N=3/group) using the CellTiter 96® AQueous One Solution Cell Proliferation Assay (MTS, Promega Inc, USA) at day 7 of culture. Briefly, at day 7, the samples were transferred to new wells, washed twice with PBS, and then incubated with 200 μL of the MTS solution (40 uL MTS solution, 160 uL growth media) for 2 hours at 37° C. Next, 100 μL from the supernatant of each sample was transferred to 96-well plates, and the absorbance was read at 490 nm using a plate reader. To eliminate the interface of MTS reagent with the samples, acellular scaffolds were incubated with the MTS reagent, and their absorbance values were deducted from the absorbance values measured for each cellular scaffold. FIG. 24 illustrates the process employed for assessing cell viability and growth.

Furthermore, all quantitative data were expressed as mean±standard deviation. All statistical analyses were performed using the statistical software Prism GraphPad version 8 (GraphPad, USA). Statistical analyses were performed using the one-way analysis of variance (one-way ANOVA) with Tukey's post hoc test. Statistical significance was evaluated at *p<0.05, **p<0.01, ***p<0.001, and ****p<0.0001.

The outcomes of the in vitro experiments conducted to characterize the fabricated scaffolds with 30%, 40%, and 50% pore volumes are discussed hereinafter. The analysis encompassed scanning electron microscopy, mechanical testing, biodegradation assessment, and biocompatibility evaluations (cell growth and viability). It presented the collected data and offers a comprehensive analysis and discussion of the results obtained.

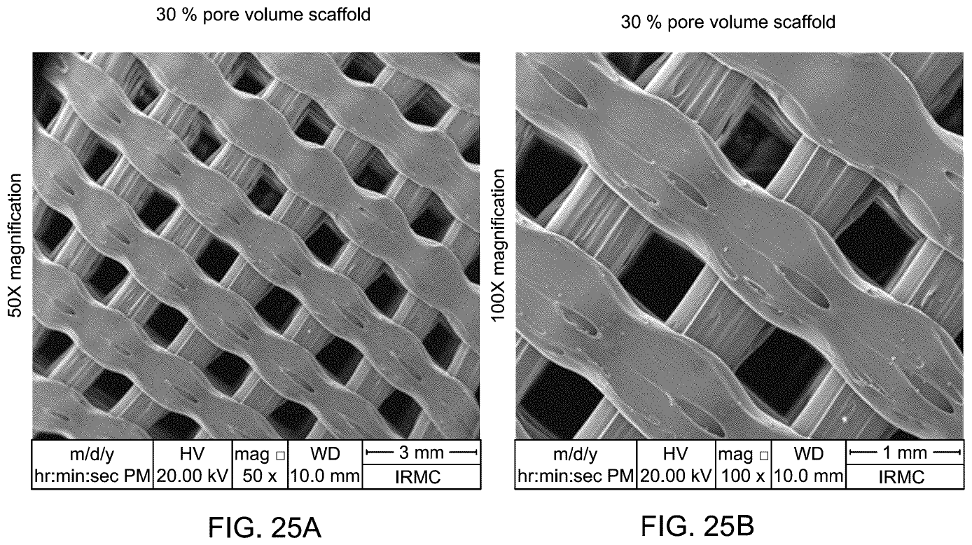

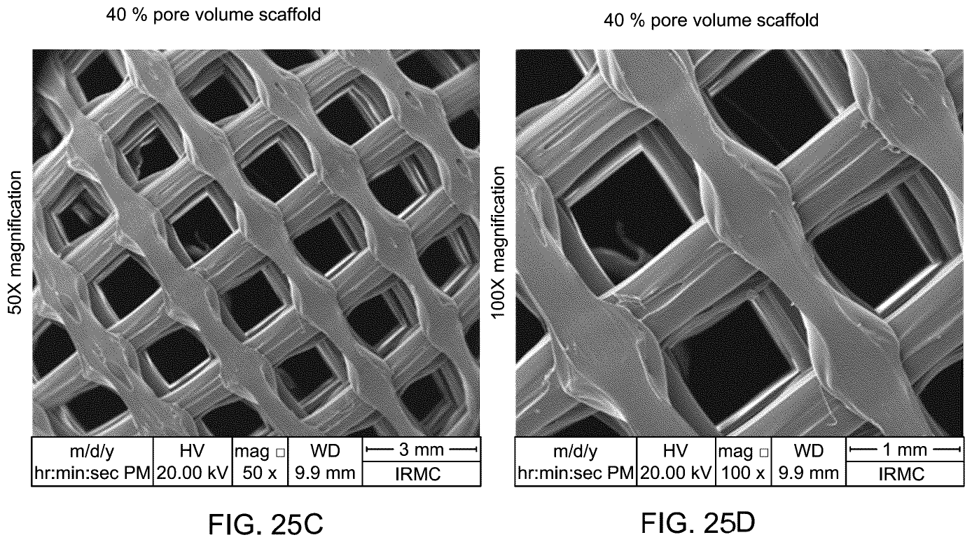

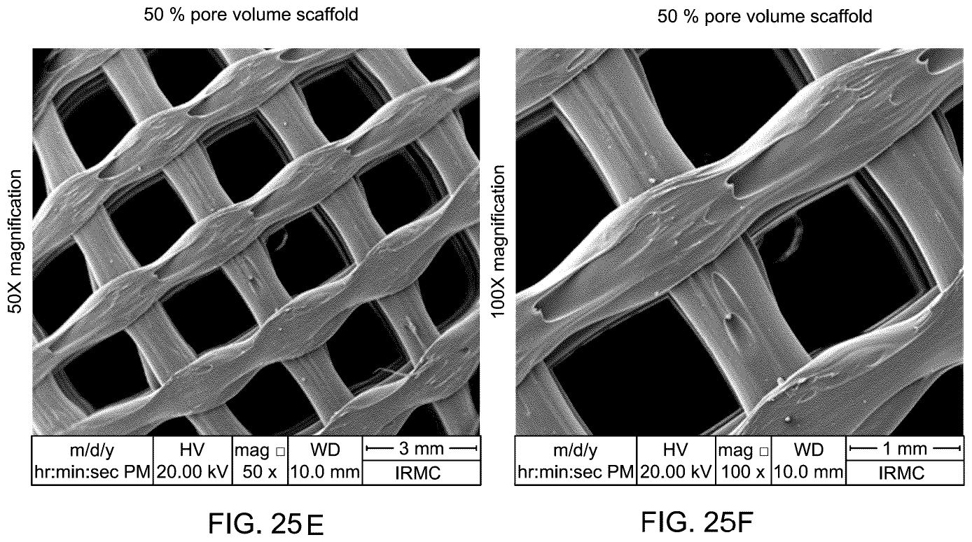

Scanning electron microscopy (SEM) analysis was employed to examine the microstructure and variation in pore sizes within the fabricated scaffolds. FIGS. 25A-25F shows the SEM images of the 30%, 40%, and 50% scaffolds captured at 50× and 100× magnifications. These scaffolds were fabricated using 3D printing techniques, specifically fused deposition modeling (FDM). The SEM images reveal that these scaffolds maintain a highly porous microstructure and 3D interconnectivity, which is a crucial feature to ensure facilitating nutrient and oxygen delivery, waste exchange, cell infiltration, and vascular ingrowth throughout the scaffold in vivo.

Qualitatively, it was evident that the pore size in the 50% pore volume scaffold group was the largest, followed by the 40% pore volume scaffold, with the 30% pore volume scaffold group exhibiting the smallest pores. These qualitative findings were further confirmed quantitatively by measuring the pore size in each scaffold group using the ImageJ software. Raw data of the bone scaffolds is provided below in Table 5 (below). Quantitative pore size analysis revealed that the pore size was the highest in the 50% pore volume scaffold group, followed by the 40% and 30% pore volume scaffold groups (as shown in FIG. 25G). Statistically, the pore size was significantly higher in the 50% and 40% pore volume scaffold groups compared to the 30% pore volume scaffold group. However, no statistically significant differences were observed between the 50% and 40% pore volume scaffold groups. Overall, the observed variations in pore size among the scaffold groups was an indication of the printing accuracy.

TABLE 5
Raw Data of microstructural analysis of the scaffold groups.

| Scaffold Pore | | Pore Size | | Standard |
|---|---|---|---|---|
| volume | Trials | [mm] | Mean | Deviation |
| 30% | 1 | 0.533 | 0.530 | 0.016 |
| | 2 | 0.538 | | |
| | 3 | 0.525 | | |
| | 4 | 0.503 | | |
| | 5 | 0.552 | | |
| 40% | 1 | 1.015 | 0.956 | 0.106 |
| | 2 | 1.016 | | |
| | 3 | 0.992 | | |
| | 4 | 0.744 | | |
| | 5 | 1.013 | | |
| 50% | 1 | 1.049 | 1.030 | 0.022 |
| | 2 | 1.029 | | |
| | 3 | 1.015 | | |
| | 4 | 0.998 | | |
| | 5 | 1.060 | | |

Indeed, bone tissue engineering scaffolds may need to display high mechanical strength to provide adequate support during the healing process. Additionally, introducing a high pore volume into the scaffold's structure is essential to enable enhanced cell infiltration, vascularization, and bone ingrowth and regeneration. However, it is important to note that while increasing pore volume is beneficial for tissue regeneration, it may impact the mechanical properties of the scaffold. Therefore, a careful balance between mechanical strength and porosity may need to be investigated and determined during the design process to ensure optimal scaffold performance. While bone scaffolds with 50% porosity may display enhanced cellular infiltration, vascularization, and bone regeneration; they may not be able to withstand the harsh mechanical loads being applied in the bony environment. Likewise, bone scaffolds with 30% porosity may indeed display adequate mechanical resilience, but may not promote sufficient cellular infiltration, vascularization, and bone regeneration as shown in previous research. Therefore, it is believed that a bone scaffold with 40% porosity may achieve a balance between mechanical strength and facilitating enhanced cellular infiltration. Yet, further in vitro mechanical validation may be required to confirm this hypothesis, as discussed in the following paragraphs.

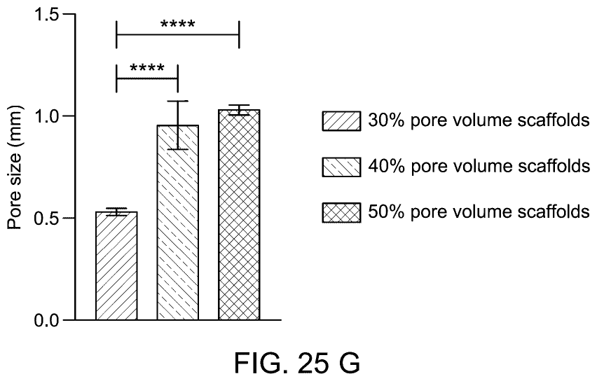

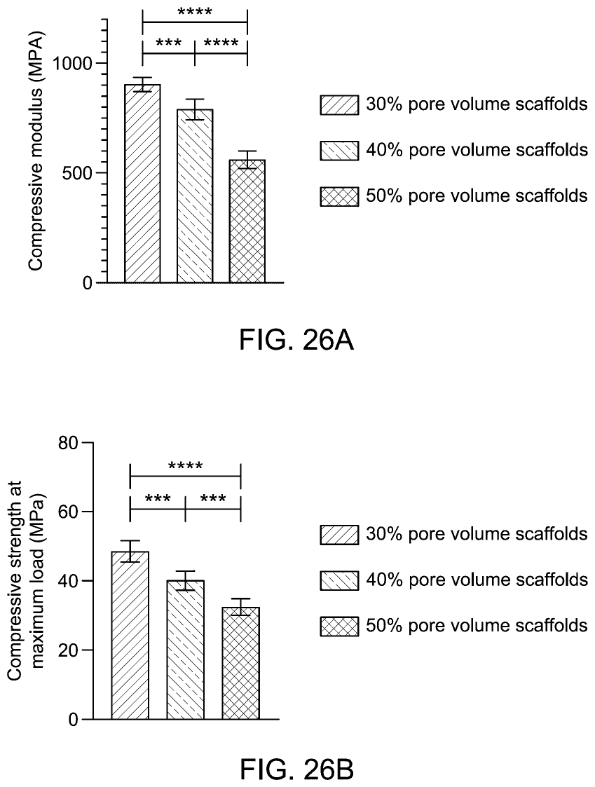

Compression mechanical testing was conducted on the various scaffold groups to assess their mechanical strength, given their intended placement in a load-bearing anatomical site. FIGS. 26A and 26B present the recorded values of compressive modulus and compressive strength for the 30%, 40%, and 50% pore volume scaffolds following the application of compression force, accompanied by the performed statistical analysis. Compressive stress refers to the maximum load an object can withstand before initiating breakage, while compressive modulus indicates the maximum load the object can withstand before complete failure [C. E. Misch, Z. Qu, and M. W. Bidez, “Mechanical properties of trabecular bone in the human mandible: implications for dental implant treatment planning and surgical placement,” J. Oral Maxillofac. Surg. Off. J. Am. Assoc. Oral Maxillofac. Surg., vol. 57, no. 6, pp. 700-706; discussion 706-708, June 1999, incorporated herein by reference in its entirety]. Results indicate that increasing the scaffold's pore volume from 30% to 50% led to a reduction in overall mechanical strength. Specifically, the average compressive modulus values for scaffolds with pore volumes of 30%, 40%, and 50% were 903.28 MPa, 790.27 MPa, and 560.2651 MPa, respectively. Additionally, the average compressive strength values for the same groups were 48.48304 MPa, 40.09623 MPa, and 32.401432 MPa, respectively. These values fall within the same range as those reported for human bone, indicating their suitability for application in boney load bearing applications.

These findings are in line with numerical values acquired from finite element analysis (FEA) conducted earlier, which demonstrated a decrease in scaffold's overall mechanical strength with increasing pore volume. Tables 6-8 (below) provide raw Data of the mechanical properties of the scaffold groups. FIGS. 26A and 26B further present the statistical analysis of compressive modulus and compressive strength of the different scaffold groups, respectively. Significant differences were observed among all scaffold groups, indicating a substantial enhancement in scaffold capability to withstand mechanical forces and maintain structural integrity as pore volume decreases. This improvement in mechanical properties with decreasing pore volume is attributed to the fact that as the pore volume decreases, more material occupies the scaffold volume and less empty space, enabling the scaffold to tolerate greater compression forces. Regardless of the variation in strength among the different scaffold groups, all scaffolds demonstrated the ability to withstand loads significantly exceeding 800 N (average human weight) without failure. Overall, these data indicate the suitability of all scaffold groups for the intended load-bearing applications.

TABLE 6
Raw Data of the mechanical properties of the 30% pore volume scaffold.

| | Compressive | | Compressive | Extension | | |
|---|---|---|---|---|---|---|
| | Strength at | | strain at | at | Load | Energy |
| | Maximum | Compressive | Maximum | Maximum | at | at |
| | Load | Modulus | Load | Load | Break | Break |
| Specimen | [MPa] | [MPa] | [mm/mm] | [mm] | [N] | [J] |
| 1 | 46.60519 | 926.5665 | 0.12954 | 1.29771 | 3812.878 | 0.17675 |
| 2 | 44.53454 | 863.523 | 0.1294 | 1.30021 | 3643.475 | 0.1674 |
| 3 | 47.56378 | 927.2706 | 0.12779 | 1.29271 | 3889.499 | 0.18118 |
| 4 | 51.96281 | 925.5054 | 0.13004 | 1.30021 | 4251.197 | 0.20073 |
| 5 | 52.53202 | 915.5102 | 0.12967 | 1.29812 | 4297.765 | 0.18873 |
| 6 | 47.69993 | 861.3064 | 0.12988 | 1.29917 | 3902.441 | 0.16769 |
| Mean | 48.48304 | 903.2804 | 0.129387 | 1.298022 | 3966.209 | 0.180413 |
| Standard | 2.860019 | 29.16549 | 0.000744 | 0.002557 | 234.082 | 0.011741 |
| Deviation | | | | | | |

TABLE 7
Raw Data of the mechanical properties of the 40% pore volume scaffold.

| | Compressive | | Compressive | Extension | | |
|---|---|---|---|---|---|---|
| | Strength at | | strain at | at | Load | Energy |
| | Maximum | Compressive | Maximum | Maximum | at | at |
| | Load | Modulus | Load | Load | Break | Break |
| Specimen | [MPa] | [MPa] | [mm/mm] | [mm] | [N] | [J] |
| 1 | 40.4208 | 816.2369 | 0.12996 | 1.30021 | 3306.92 | 0.1695 |
| 2 | 38.06894 | 722.0519 | 0.13004 | 1.30021 | 3114.507 | 0.13437 |
| 3 | 36.5165 | 833.8281 | 0.13004 | 1.30021 | 2987.5 | 0.14593 |
| 4 | 39.35993 | 836.8454 | 0.13004 | 1.30021 | 3220.127 | 0.15513 |
| 5 | 41.94967 | 748.7975 | 0.13008 | 1.30021 | 3432 | 0.16032 |
| 6 | 44.26152 | 783.8644 | 0.12683 | 1.28417 | 3620.472 | 0.17139 |
| Mean | 40.09623 | 790.2707 | 0.129498 | 1.297537 | 3280.255 | 0.156107 |
| Standard | 2.528395 | 43.11915 | 0.001194 | 0.005978 | 206.6714 | 0.01296 |
| Deviation | | | | | | |

TABLE 8
Raw Data of the mechanical properties of the 50% pore volume scaffold.

| | Compressive | | Compressive | Extension | | |
|---|---|---|---|---|---|---|
| | Strength at | | strain at | at | Load | Energy |
| | Maximum | Compressive | Maximum | Maximum | at | at |
| | Load | Modulus | Load | Load | Break | Break |
| Specimen | [MPa] | [MPa] | [mm/mm] | [mm] | [N] | [J] |
| 1 | 34.57358 | 523.9476 | 0.12975 | 1.29875 | 2828.545 | 0.13417 |
| 2 | 34.68439 | 609.9996 | 0.13004 | 1.30021 | 2837.609 | 0.14305 |
| 3 | 31.4323 | 535.7506 | 0.13004 | 1.30021 | 2571.549 | 0.11999 |
| 4 | 33.81756 | 584.6861 | 0.13004 | 1.30021 | 2766.693 | 0.1299 |
| 5 | 31.53667 | 590.7095 | 0.13004 | 1.30021 | 2580.088 | 0.12736 |
| 6 | 28.36409 | 516.4971 | 0.13004 | 1.30021 | 2320.532 | 0.10566 |
| Mean | 32.40143 | 560.2651 | 0.129992 | 1.299967 | 2650.836 | 0.126688 |
| Standard | 2.233187 | 36.13012 | 0.000108 | 0.000544 | 182.7019 | 0.011701 |
| Deviation | | | | | | |

Further, a biodegradation assessment was implemented to assess the rate of biodegradation of implants, facilitating the evaluation and selection of biomaterials for bone scaffolds. Ideally, these materials should exhibit slow degradation rates during the early healing stages (first 2-4 months) to maintain their mechanical strength and structural integrity and degrade in proportion to the rate of new bone formation. The biodegradation rate of all scaffold groups under physiological conditions (37° C. and gentle agitation) were investigated to determine if scaffold pore volume affects the biodegradation rate. This involved measuring the initial and final weights of the samples and calculating the weight loss percentage using Equation 1, where Wi is the initial weight and W is the final weight:

$$Biodegradation \% = \frac{W i - W f}{W i} \times 100 \qquad Equation ( 1 )$$

Table 9 provides raw data of the biodegradation test of the scaffold groups. The results revealed that after two months, the weight loss percentage for all scaffold groups was approximately 0.54%, demonstrating the suitability of the selected material for the intended application (as depicted in FIG. 27). Furthermore, no statistically significant differences were observed between the groups, suggesting that pore volume does not influence the scaffold biodegradation rate. Within just two months of implanting the scaffolds, regardless of pore volume, the degradation rate remained relatively stable since they were composed of the same material. Moreover, the observed degradation rate over this period is advantageous, as substantial bone growth has not yet occurred [M. Laubach et al., “Clinical translation of a patient-specific scaffold-guided bone regeneration concept in four cases with large long bone defects,” J. Orthop. Transl., vol. 34, pp. 73-84, May 2022, incorporated herein by reference in its entirety]. Significant weight loss from the scaffolds during this timeframe could compromise their mechanical strength, potentially leading to implant failure. Typically, the degradation rate of PLA-fabricated bone scaffolds during the initial six months is relatively slow [P. Feng, J. Jia, 5 M. Liu, S. Peng, Z. Zhao, and C. Shuai, “Degradation mechanisms and acceleration strategies of poly(lactic acid) scaffold for bone regeneration,” Mater. Des., vol. 210, p. 110066, November 2021, incorporated herein by reference in its entirety]. However, as the scaffolds approach their breakdown point, the biodegradation process accelerates due to the increased ease of breaking bonds and polymers. By this time, a reliable amount of bone should have grown, reinforcing the mechanical integrity of the defect site along with the scaffold remnants until complete bone formation occurs.

TABLE 9
Raw Data of the Biodegradation test of the scaffold groups.

| Scaffold | | Initial | Final | Weight | |
|---|---|---|---|---|---|
| Pore | | Weight | Weight | Loss | Average |
| volume | Specimen | [mg] | [mg] | % | % |
| 30% | 1 | 855.7 | 851.9 | 0.444 | 0.544 |
| | 2 | 856.99 | 851.22 | 0.673 | |
| | 3 | 856.32 | 851.92 | 0.514 | |
| 40% | 1 | 760.86 | 756.93 | 0.517 | 0.531 |
| | 2 | 751.03 | 747 | 0.537 | |
| | 3 | 757.28 | 753.19 | 0.540 | |
| 50% | 1 | 644.39 | 641.05 | 0.518 | 0.543 |
| | 2 | 653.64 | 650.07 | 0.546 | |
| | 3 | 644.22 | 640.59 | 0.563 | |

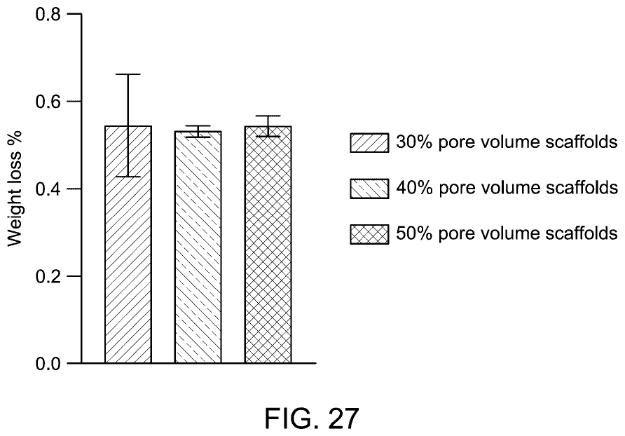

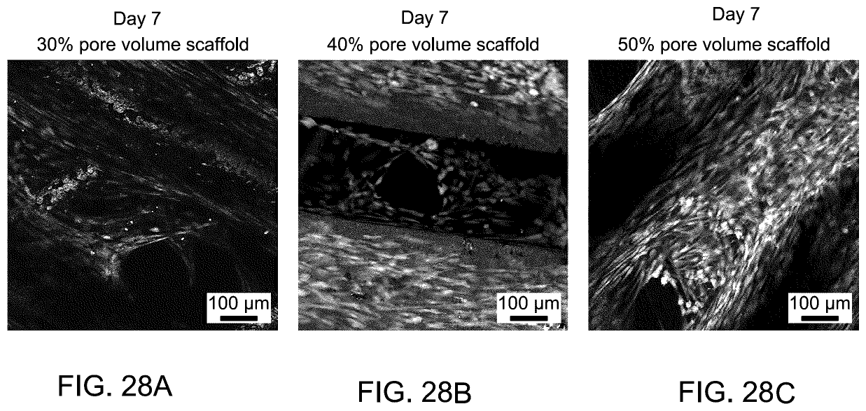

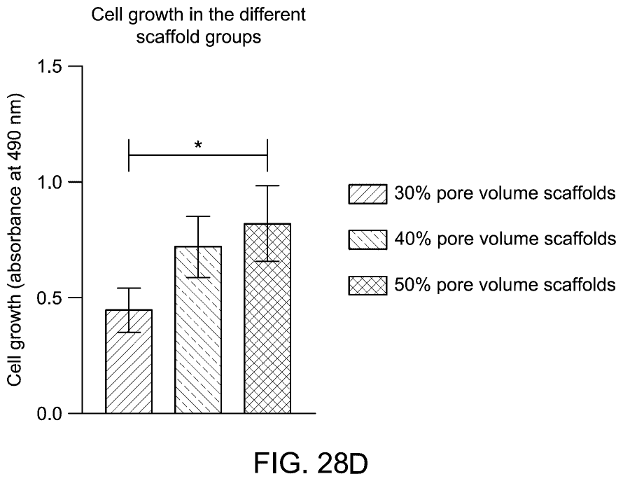

Further, biocompatibility evaluations were implemented. Scaffold biocompatibility is a critical consideration in bone tissue engineering, predominantly influenced by material selection and scaffold design [A. G. Abdelaziz et al., “A Review of 3D Polymeric Scaffolds for Bone Tissue Engineering: Principles, Fabrication Techniques, Immunomodulatory Roles, and Challenges,” Bioengineering, vol. 10, no. 2, Art. no. 2, February 2023, incorporated herein by reference in its entirety]. Herein, PLA was utilized as the primary material across all scaffold groups due to its established biocompatibility. However, the key difference among these groups was the variance in scaffold pore volume. Generally, scaffolds with larger pore volumes support enhanced cell growth and infiltration into their cores, whereas those with smaller pore volumes often exhibit limited cell infiltration and growth. The investigation involved fabricating scaffolds with varying pore volumes of 30%, 40%, and 50%, aiming to examine the impact of pore volume in the scaffold design on cell viability, infiltration, and growth. Table 10 (below) provides raw data of the cell growth of the scaffold groups. For biological evaluations, human Bone marrow-derived stem cells (hBMSCs) were used due to their relevance and widespread use in bone tissue engineering research, given their role in bone tissue formation. Cell viability and growth assessments were conducted by culturing hBMSCs on all scaffold groups for 7 days. The viability assessment primarily focused on assessing the infiltration of cells into the scaffold cores using the live/dead assay. Notably, greater cell infiltration was observed in the 40% and 50% scaffold groups compared to the 30% group (as depicted in FIGS. 28A-28C). In the 30% scaffold group, minimal cell presence was noted at the core, accompanied by some dead cells, likely attributed to inadequate oxygen and nutrient delivery due to smaller pore size, as indicated by SEM analysis (as shown in FIGS. 25A-25G). Conversely, by day 7, cells had successfully infiltrated the cores of the 40% and 50% scaffold groups, maintaining viability possibly due to larger pore sizes facilitating sufficient oxygen and nutrient delivery, as indicated by SEM analysis. These results also further validate the biocompatibility of the chosen material. In addition to viability evaluations, growth analysis using an MTS assay revealed cell growth was the highest in the 50% group, followed by the 40% and then the 30% groups (as depicted in FIG. 28D). Statistically, the cell growth was significantly higher in the 50% scaffold groups compared to the 30% scaffold group. However, no statistically significant difference was observed between the 40% and 50% groups, indicating similar support for cell growth. In conclusion, while the 50% scaffold group demonstrated the highest cell growth, the lack of significant difference between it and the 40% scaffold group suggests that the 40% scaffold group may be more suitable for load-bearing bone tissue engineering applications. This preference stems from the 40% group exhibiting significantly higher mechanical strength compared to the 50% group, highlighting its potential superiority in supporting the demands of load-bearing scenarios.

TABLE 10
Raw Data of the cell growth of the scaffold groups.

| Scaffold Pore | | Cell | Average |
|---|---|---|---|
| volume | Specimen | Growth | % |
| 30% | 1 | 0.346 | 0.446667 |
| | 2 | 0.46 | |
| | 3 | 0.534 | |
| 40% | 1 | 0.73 | 0.722667 |
| | 2 | 0.586 | |
| | 3 | 0.852 | |
| 50% | 1 | 0.639 | 0.822 |
| | 2 | 0.869 | |
| | 3 | 0.958 | |

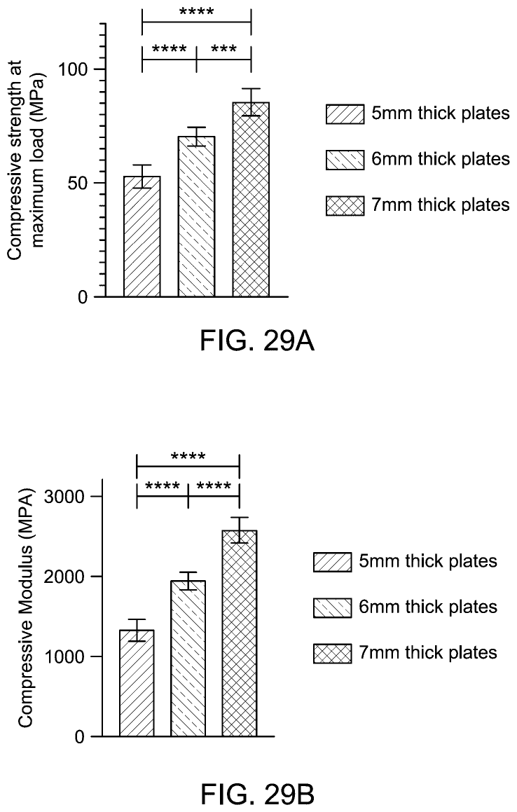

Further, the outcomes of the in vitro experiments conducted to characterize the fabricated plates with 5 mm, 6 mm, and 7 mm thicknesses are presented in the following paragraphs. The analysis encompasses mechanical testing and biodegradation assessment. Further, the experiments presents the collected data and offers a comprehensive analysis and discussion of the results obtained. The mechanical analysis of bone plates was conducted using the four-point bending method, a standard approach mandatory by ASTM F382 for assessing the mechanical properties of bone plates [F. A. Bologna, A. L. Audenino, and M. Terzini, “Bone Plates Runout Prediction Through Tensile Strength and Geometric Properties for Regulatory Mechanical Testing,” Ann. Biomed. Eng., vol. 52, no. 2, pp. 239-249, February 2024, incorporated herein by reference in its entirety]. This method involves subjecting the specimen to bending forces at two distinct points, creating a central region where deformation occurs while maintaining support at both ends (as shown in FIG. 22B). This setup allows for a more controlled assessment of mechanical behavior compared to other bending tests, making it particularly suitable for evaluating the strength and stiffness of materials, including bone plates. Following the four-point bending mechanical test, data on compressive strength and compressive modulus were recorded for plates with varying thicknesses of 5 mm, 6 mm, and 7 mm. Statistical analysis was then performed to interpret the results. In terms of compressive strength at maximum load, thicker plates exhibited higher mean values. Specifically, the 7 mm plate thickness showed the highest mean compressive strength at a maximum load of 85.39 MPa, followed by the 6 mm plate thickness at 70.19 MPa, with the 5 mm plate thickness displaying the lowest mean value of 52.89 MPa. This trend suggests that increasing plate thickness enhances the plates' ability to resist deformation and failure under compressive forces. Similarly, the compressive modulus demonstrated a parallel trend, with thicker plates showing higher mean values, indicating increased stiffness and resistance to elastic deformation. The 7 mm plate thickness recorded the highest mean compressive modulus of 2577.97 MPa, followed by the 6 mm plate thickness at 1941.85 MPa, and the 5 mm plate thickness at the lowest mean value of 1331.08 MPa. These results imply that augmenting plate thickness enhances their load-bearing capacity and structural integrity. Tables 11-13 (below) provide raw data of the bone plates. Further, FIGS. 29A and 29B illustrate the statistical analysis of compressive stress and compressive modulus for different plate groups, respectively, revealing significant differences between all plate groups (5 mm vs. 6 mm, 6 mm vs. 7 mm, and 5 mm vs. 7 mm thickness). This highlights the importance of increasing these parameters to strengthen plates' ability to withstand mechanical forces and loads. In conclusion, this highlights that augmenting bone plate thickness positively impacts their mechanical strength. Thicker plates exhibit higher compressive stress and compressive modulus, suggesting improved resistance to deformation and increased load-bearing capacity. Additionally, these findings are in line with numerical values acquired from finite element analysis (FEA) conducted earlier, which demonstrated an increase in plate's overall mechanical strength with increasing the plate thickness.

TABLE 11
Raw Data of the mechanical properties of the 5 mm thick plates.

| | | Compressive | |
|---|---|---|---|
| | Compressive | Strength at | Load at |
| | Modulus | Maximum | Break |
| Specimen | [MPa] | Load [MPa] | [N] |
| 1 | 1407.583 | 55.93416 | 2796.708 |
| 2 | 1361.684 | 54.11022 | 2705.511 |
| 3 | 1514.682 | 60.19002 | 3009.501 |
| 4 | 1198.486 | 47.6251 | 2381.255 |
| 5 | 1305.584 | 51.88096 | 2594.048 |
| 6 | 1198.486 | 47.6251 | 2381.255 |
| Mean | 1331.084 | 52.89426 | 2644.713 |
| Standard | 112.7767 | 4.48149 | 224.075 |
| Deviation | | | |

TABLE 12
Raw Data of the mechanical properties of the 6 mm thick plates.

| | | Compressive | |
|---|---|---|---|
| | Compressive | Strength at | Load at |
| | Modulus | Maximum | Break |
| Specimen | [MPa] | Load [MPa] | [N] |
| 1 | 1822.24 | 65.8645 | 3293.225 |
| 2 | 1939.985 | 70.12036 | 3506.018 |
| 3 | 1996.054 | 72.14696 | 3607.348 |
| 4 | 1945.592 | 70.32302 | 3516.151 |
| 5 | 1833.454 | 66.26982 | 3313.491 |
| 6 | 2113.799 | 76.40282 | 3820.141 |
| Mean | 1941.854 | 70.18791 | 3509.396 |
| Standard | 98.80796 | 3.571393 | 178.570 |
| Deviation | | | |

TABLE 13
Raw Data of the mechanical properties of the 7 mm thick plates.

| | | Compressive | |
|---|---|---|---|
| | Compressive | Strength at | Load at |
| | Modulus | Maximum | Break |
| Specimen | [MPa] | Load [MPa] | [N] |
| 1 | 2453.562 | 81.26666 | 4063.333 |
| 2 | 2520.867 | 83.49592 | 4174.796 |
| 3 | 2435.207 | 80.65868 | 4032.934 |
| 4 | 2643.239 | 87.54912 | 4377.456 |
| 5 | 2912.458 | 96.46616 | 4823.308 |
| 6 | 2502.511 | 82.88794 | 4144.397 |
| Mean | 2577.974 | 85.38741 | 4269.371 |
| Standard | 163.7863 | 5.424915 | 271.246 |
| Deviation | | | |

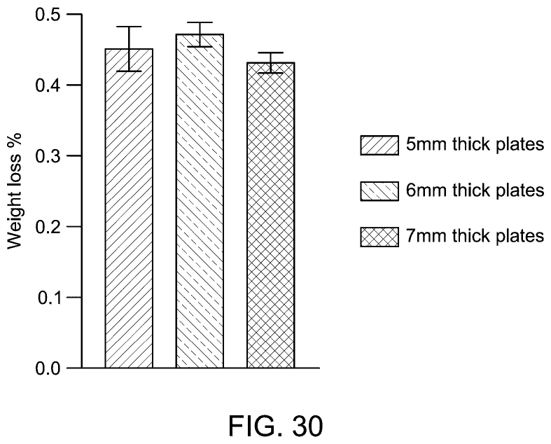

Further, biodegradation assessments were employed. The biodegradation test was conducted on the plate samples following the same protocol as the scaffold assessment. Table 14 (below) provides raw data of the biodegradation test of the plate groups. The results shown in FIG. 30 indicate that the average degradation rates for plates with thicknesses of 5 mm, 6 mm, and 7 mm are 0.45%, 0.47%, and 0.43%, respectively. Notably, there were no statistically significant differences observed among the plate groups, as shown in FIG. 30. These findings suggest that the bone plate maintains its mechanical integrity during the initial stages of implantation, which is a crucial requirement for ensuring successful fracture healing, stability, and functional recovery of the affected bone during the critical healing period. They also suggest that the plate thickness does not influence the plate biodegradation rate.

TABLE 14
Raw Data of the Biodegradation test of the plate groups.

| | | Initial | Final | Weight | |
|---|---|---|---|---|---|
| Plate | | Weight | Weight | Loss | Average |
| Thickness | Specimen | [mg] | [mg] | % | % |
| 5 mm | 1 | 3139.52 | 3126.49 | 0.415 | 0.451 |
| | 2 | 3002.86 | 2989.01 | 0.46123 | |
| | 3 | 3084.96 | 3070.25 | 0.4768 | |
| 6 mm | 1 | 3751.97 | 3734.52 | 0.465 | 0.472 |
| | 2 | 3781.18 | 3762.63 | 0.491 | |
| | 3 | 3892.29 | 3874.43 | 0.459 | |
| 7 mm | 1 | 4693.23 | 4672.44 | 0.443 | 0.432 |
| | 2 | 4714.72 | 4694.14 | 0.437 | |
| | 3 | 4710.36 | 4690.79 | 0.415 | |

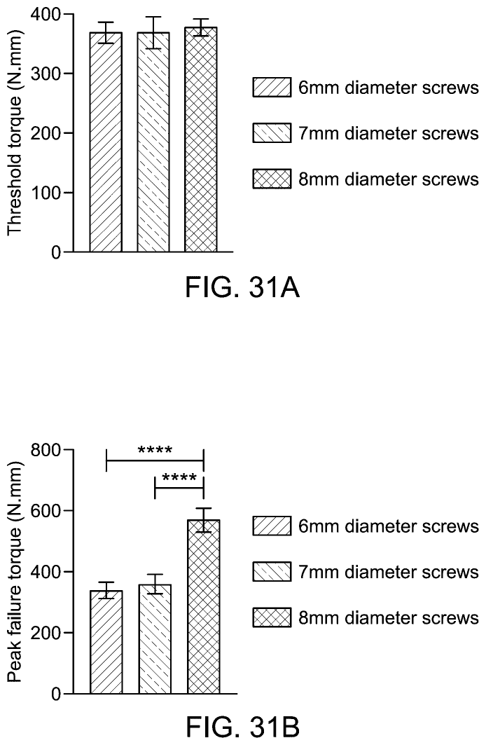

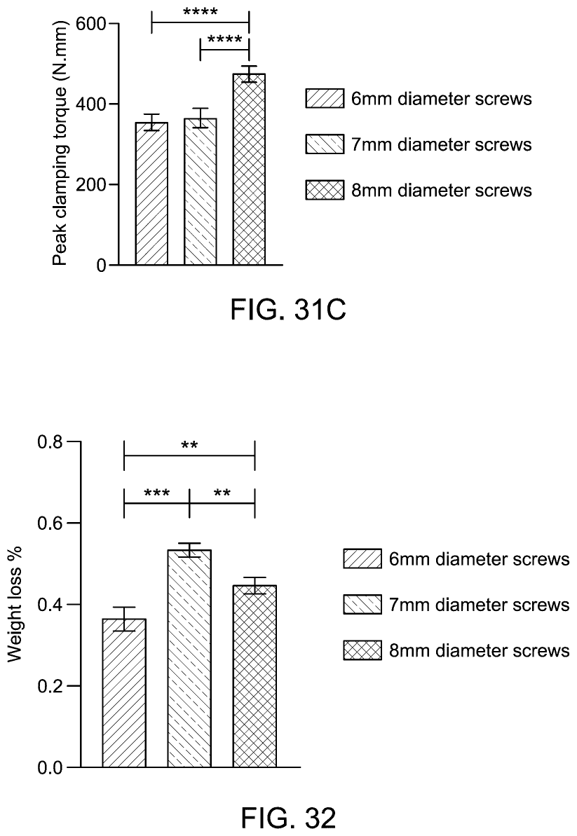

Further, bone screws characterizations were employed. The outcomes of the in vitro experiments conducted to characterize the fabricated screws with 6 mm, 7 mm, and 8 mm diameters are presented herein. The analysis encompasses mechanical testing and biodegradation assessment. It presents the collected data and offers a comprehensive analysis and discussion of the results obtained. The torsional mechanical analysis of bone screws, as mandated by ASTM F543 [ASTM F543 Standard Specification and Test Methods for Metallic Medical Bone Screws.”, incorporated herein by reference in its entirety], provides a comprehensive assessment of their ability to withstand torsion loads, crucial in orthopedic applications. This method was employed to evaluate the mechanical properties of the designed bone screws, utilizing different diameters (6 mm, 7 mm, and 8 mm). During the analysis, values of threshold torque (TT), peak failure torque (PFT), and peak clamping torque (PCT) were measured and recorded, offering significant insights into the screws' mechanical behavior. Tables 15-17 (below) provide raw data of the mechanical properties of bone screws. The results are depicted in FIGS. 31A-31C. TT represents the minimum torque required for proper fixation and stability of the bone screw, while PFT is the maximum torque a screw can withstand under torsion before it starts failing. Typically, the PFT value needs to surpass the TT value to prevent failure. In the torsion test results (as shown in FIG. 31A), TT values were determined to be 368.6224 N·mm, 368.4719 N·mm, and 377.3779 N·mm for 6 mm, 7 mm, and 8 mm diameter screws, respectively. Although no statistically significant difference was observed between screw groups, it is essential to note the relevance of TT in ensuring screw stability. Moving to PFT values (as shown in FIG. 31B), screws with diameters of 6 mm, 7 mm, and 8 mm exhibited PFT values of 338.9238 N·mm, 359.6666 N·mm, and 570.6356 N·mm, respectively. Significant differences were noted between 6 mm and 8 mm screws, and between 7 mm and 8 mm screws. Only the 8 mm screw could withstand applied torsion loads without failure, evidenced by its PFT exceeding the TT value. Earlier finite element analysis (FEA) simulations corroborated these findings, indicating that only 8 mm diameter screws may be able to resist torsion without failure. The experimental results aligned well with the FEA predictions. Furthermore, peak clamping torque (PCT) values (as shown in FIG. 31C) were assessed to determine the maximum torque required for optimal compression between bone and screws, essential for promoting stability and healing. Screws with 6 mm, 7 mm, and 8 mm diameters recorded PCT values of 353.7731 N·mm, 381.9641 N·mm, and 474.0068 N·mm, respectively. Again, the 8 mm screw achieved optimal compression without failure, supported by its PCT value being lower than the PFT value. In conclusion, the torsional mechanical analysis provided valuable insights into the performance of bone screws. Only the 8 mm diameter screw demonstrated sufficient mechanical strength to withstand torsion loads without failure, consistent with FEA predictions. These findings highlight the importance of selecting appropriate screw dimensions to ensure optimal stability and fracture healing in orthopedic applications.

TABLE 15
Raw Data of the mechanical properties
of the 6 mm diameter screws.

| | | Peak | Peak |
|---|---|---|---|
| | Threshold | Failure | Clamping |
| | Torque | Torque | Torque |
| Specimen | [N · mm] | [N · mm] | [N · mm] |
| 1 | 346.6726 | 322.8564 | 334.765 |
| 2 | 361.7675 | 348.0094 | 354.888 |
| 3 | 386.3365 | 387.9254 | 387.131 |
| 4 | 377.5446 | 328.9032 | 353.224 |
| 5 | 351.953 | 332.2945 | 342.124 |
| 6 | 387.46 | 313.5536 | 350.507 |
| Mean | 368.6224 | 338.9238 | 353.773 |
| Standard | 16.09815 | 24.25515 | 16.442 |
| Deviation | | | |

TABLE 16
Raw Data of the mechanical properties
of the 7 mm diameter screws.

| | | Peak | Peak |
|---|---|---|---|
| | Threshold | Failure | Clamping |
| | Torque | Torque | Torque |
| Specimen | [N · mm] | [N · mm] | [N · mm] |
| 1 | 374.9736 | 338.0924 | 356.533 |
| 2 | 355.6687 | 398.2454 | 376.957 |
| 3 | 387.0297 | 340.2851 | 363.658 |
| 4 | 376.5632 | 389.385 | 382.974 |
| 5 | 321.7122 | 322.9476 | 322.330 |
| 6 | 394.8839 | 369.0443 | 381.964 |
| Mean | 368.4719 | 359.6666 | 364.0693 |
| Standard | 24.1527 | 27.8353 | 20.988 |
| Deviation | | | |

TABLE 17
Raw Data of the mechanical properties
of the 8 mm diameter screws.

| | | | Peak |
|---|---|---|---|
| | Threshold | Peak Failure | Clamping |
| | Torque | Torque | Torque |
| Specimen | [N · mm] | [N · mm] | [N · mm] |
| 1 | 387.8562 | 592.2696 | 490.063 |
| 2 | 357.7693 | 601.3874 | 479.578 |
| 3 | 390.2845 | 593.5871 | 491.936 |
| 4 | 375.6593 | 499.3852 | 437.522 |
| 5 | 389.0039 | 558.2856 | 473.645 |
| 6 | 363.6942 | 578.8987 | 471.297 |
| Mean | 377.3779 | 570.6356 | 474.007 |
| Standard | 12.82077 | 34.73206 | 18.023 |
| Deviation | | | |

Further, biodegradation assessment (as part of bone screws characterizations) was employed. Ensuring the durability and stability of screws, coupled with controlled degradation, is crucial for maintaining stable fixation and load-bearing capacity during the critical initial 6-month period post-implantation. Minimizing degradation rates is paramount to sustain the screws' support and structural integrity, vital for successful healing and sustained implant effectiveness. Table 18 (below) provides raw data of the biodegradation test of the screw groups. The results are shown in FIG. 32, which reveal that over a two-month period, a 6 mm diameter screw experienced a weight loss of 0.36%, a 7 mm diameter screw saw a loss of 0.53%, and a 8 mm diameter screw exhibited a loss of 0.45%. These findings are promising, indicating that the screws maintained their mechanical strength and structural integrity during this early duration, ensuring proper fixation and stabilization of the bone plate. Statistical analysis conducted on the 6 mm, 7 mm, and 8 mm diameter bone screws reveals notable variations in the evaluated parameter (weight loss), as illustrated in FIG. 32. These variations suggest potential changes in performance or efficacy associated with the diverse measurements, highlighting the need for further investigation into their implications on long-term implant stability and efficacy. Furthermore, the biodegradation data indicate that the screw diameter did not significantly influence its biodegradation rate, as all screws displayed low degradation rates over the tested duration.

TABLE 18
Raw Data of the Biodegradation test of the screw groups.

| Screw | | Initial Weight | Final Weight | Weight | Average |
|---|---|---|---|---|---|
| Diameter | Specimen | [mg] | [mg] | Loss % | % |
| 6 mm | 1 | 274.28 | 273.24 | 0.379 | 0.363 |
| | 2 | 278.31 | 277.39 | 0.331 | |
| | 3 | 269.31 | 268.29 | 0.379 | |
| 7 mm | 1 | 326.6 | 324.82 | 0.545 | 0.532 |
| | 2 | 326.25 | 324.49 | 0.539 | |
| | 3 | 329.59 | 327.9 | 0.5128 | |
| 8 mm | 1 | 474.56 | 472.54 | 0.426 | 0.447 |
| | 2 | 477.18 | 474.99 | 0.459 | |
| | 3 | 474.85 | 472.68 | 0.457 | |

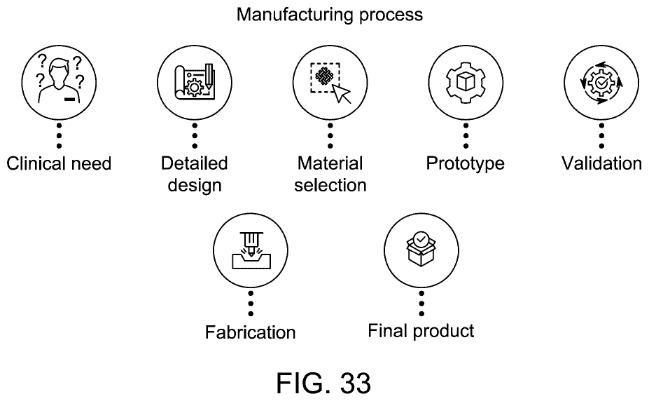

FIG. 33 illustrates intricacies of the manufacturing process, highlighting key stages and their significance in ensuring the bone substitutes, plate, and screw quality and efficiency. The manufacturing process is a critical aspect of any product's development, encompassing a series of steps that transform raw materials into finished goods. After determining the clinical need for critical-sized segmental bone fractures, a detailed design is performed to determine the exact specifications, dimensions, and structural features needed for successful bone substitutes, plates, and screw development. Materials may then be carefully selected, considering factors such as biocompatibility, biodegradability, and mechanical properties to determine the most suitable materials for the fabrication of bone substitutes, plates, and screws. Prototypes were developed to evaluate the design concept, allowing the scaffold's performance and manufacturability to be tested and improved before its real-world application. Validation was a substantial step to ensure the system's components meet the essential regulatory standards and requirements. Fabrication techniques were employed, such as 3D printing and translating design and material selections into a physical product that can be sold and used in the health field later. Finally, the final product was obtained, a fully functional, biodegradable patient-specific 3D printed bone substitutes, plates, and screws ready for use in bone regeneration applications.

The purpose of the prototype was to provide a complete representation of the final product, ensuring that all components are consistent and well-fitted with the patient's defected bone. The patient-specific biodegradable 3D models of bone substitutes, plates, and screws was successfully fabricated that contribute to an improved treatment paradigm for critical-sized segmental bone fractures. The final prototype of this complete, patient-specific treatment scheme is illustrated in FIG. 34 and is composed of the patient's fractured bone along with the fabricated patient-specific 40% pore volume bone scaffold, 7 mm thick bone plate, and 8 mm diameter bone screws. This prototype was fabricated using biodegradable polylactic acid (PLA) through 3D printing technology, using a fused deposition modeling (FDM) technique, ultimately resulting in a patient-specific and completely biodegradable system for critical size segmental bone fractures treatment.

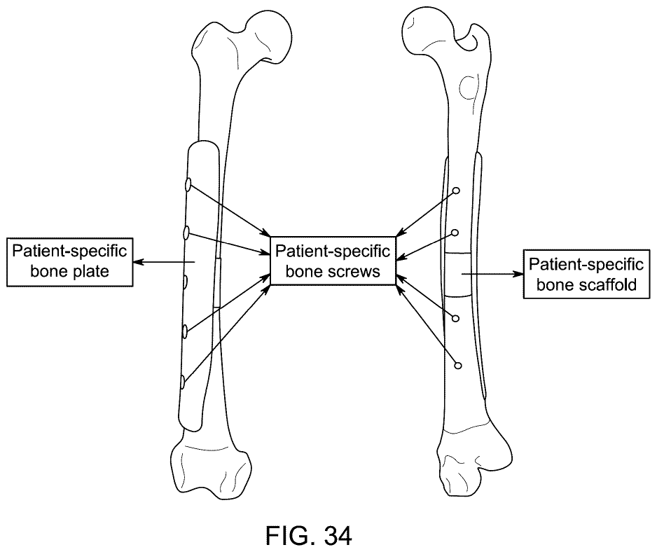

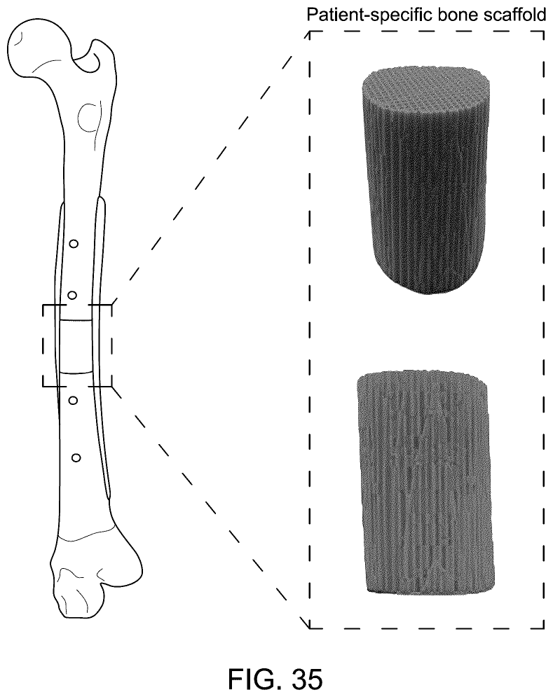

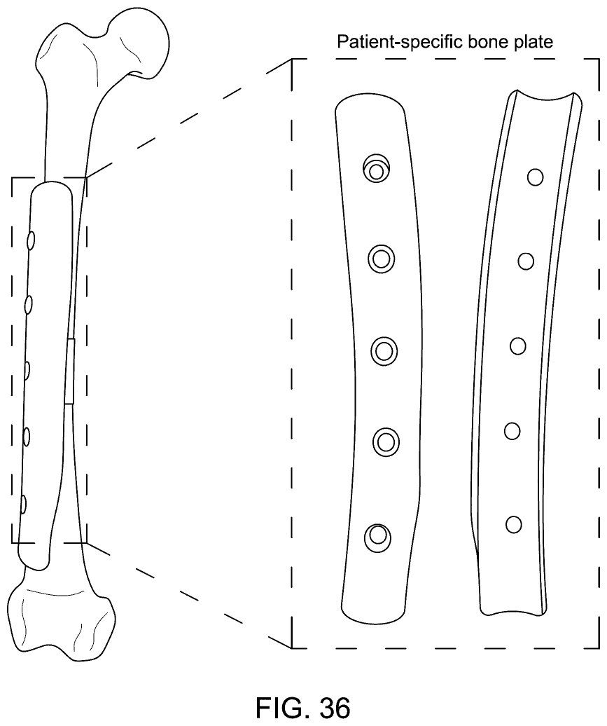

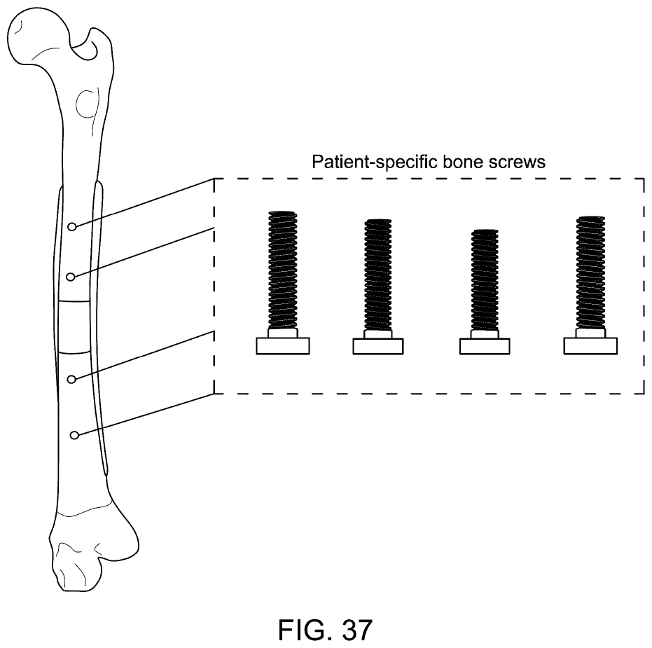

The patient-specific 40% pore volume fabricated bone scaffold is depicted in FIG. 35. This scaffold serves as a framework to support the formation of functional bone tissue by maintaining mechanical strength while offering an adequate pore volume for promoting sufficient cellular infiltration, vascularization, and bone regeneration. The patient-specific 7 mm thick fabricated bone plate is depicted in FIG. 36. This plate is used during surgical procedures to stabilize fractured bones after an injury, preserving the alignment and functionality of the bone and allowing for optimal healing. The patient-specific 8 mm diameter fabricated bone screws are depicted in FIG. 37. These screws are used during surgical procedures to secure or fixate bone plates to the skeleton structure. Additionally, the length of screws was adjusted to match the diameter of the patient's bone to prevent bone tissue irritation and ensure mechanical stability (as shown in FIG. 37).

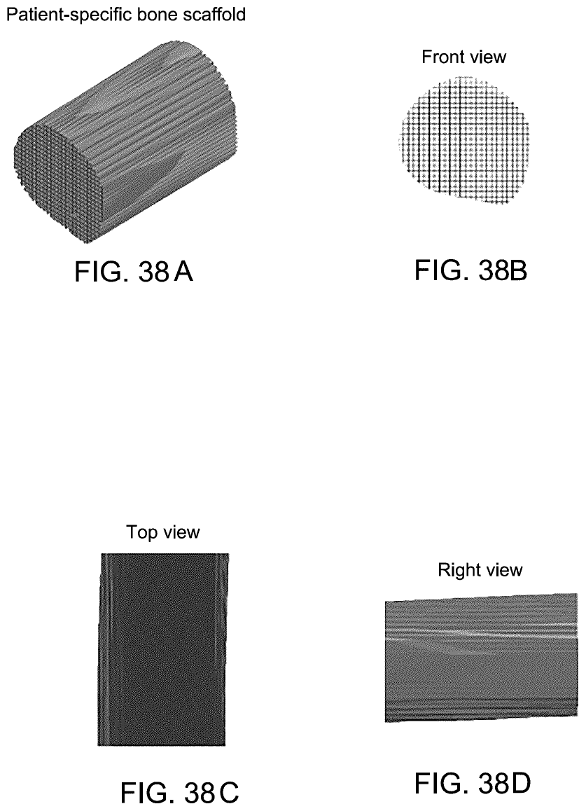

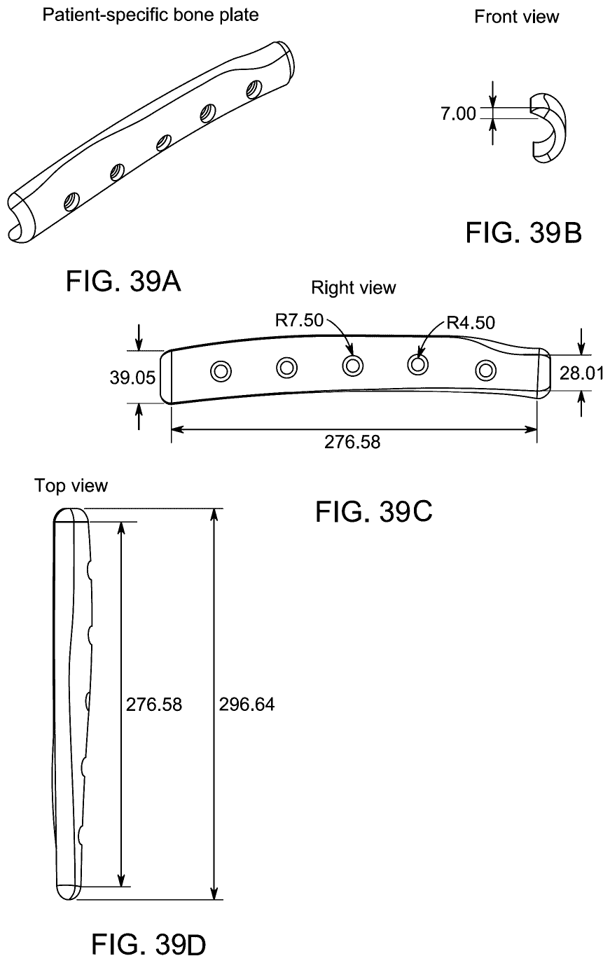

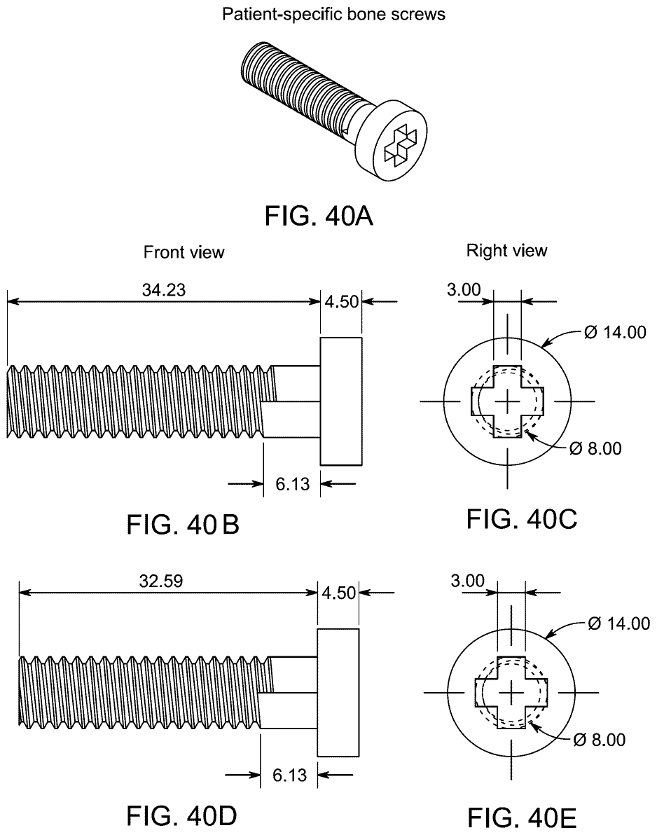

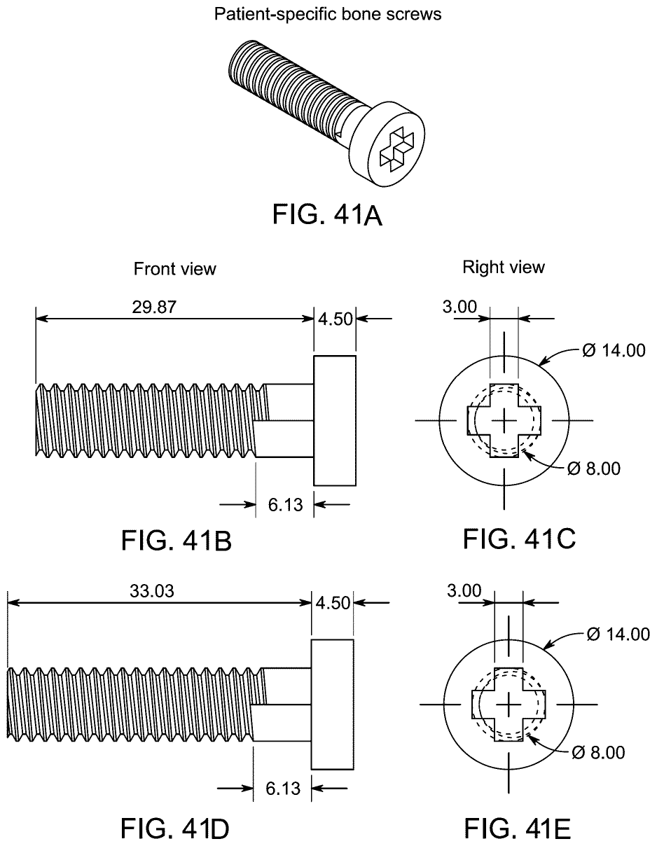

Final Engineering drawings of the designed patient-specific bone scaffold, plates, and screws are depicted in FIGS. 38-41, which include detailed drawings of scaffold with 40% pore volume, plate with a thickness of 7 mm, and screw with a diameter of 8 mm. Specifically, FIGS. 38A-38D depict final drawing of patient-specific bone scaffold with 40% pore volume. FIGS. 39A-39D depict final engineering drawing of patient-specific bone plate with 7 mm thickness. FIGS. 40A-40E depict final engineering drawing of the first and second patient-specific bone screws with 8 mm diameter. FIGS. 41A-41E depict final engineering drawing of the third and fourth patient-specific bone screws with 8 mm diameter.

The present disclosure provides a comprehensive bone repair method integrating patient-specific design with complete biodegradability. The method 100 of the present disclosure combines imaging-based fracture modeling, customized implant design, and additive manufacturing to create a bone repair implant with interconnected pore network precisely matched to patient anatomy. The bone repair implant uniquely incorporates a bone scaffold, support plate, and screws all manufactured from the same biodegradable material, with surface profiles specifically configured to match the bone fracture geometry. This integration of patient-specific design with biodegradable materials in a complete bone repair system represents an approach not previously achieved in bone fracture treatment.

The method 100 overcomes significant limitations of conventional treatments. Unlike autografts which require harvesting from a donor site and have limited availability, the bone repair implant is manufactured specifically for each patient's fracture geometry. The support plate of the bone repair implant has an inner surface profile configured to match the outer surface profile of the bone fracture in the covered area, and an outer surface profile that smoothly transitions to match the bone surface in uncovered areas, eliminating sharp edges that cause tissue irritation in traditional implants. The biodegradable material construction eliminates both the stress shielding associated with metallic implants and the need for secondary removal surgery required with non-biodegradable implants. The interconnected pore network of the bone scaffold promotes tissue integration while maintaining mechanical strength, addressing the limited integration seen with allografts.

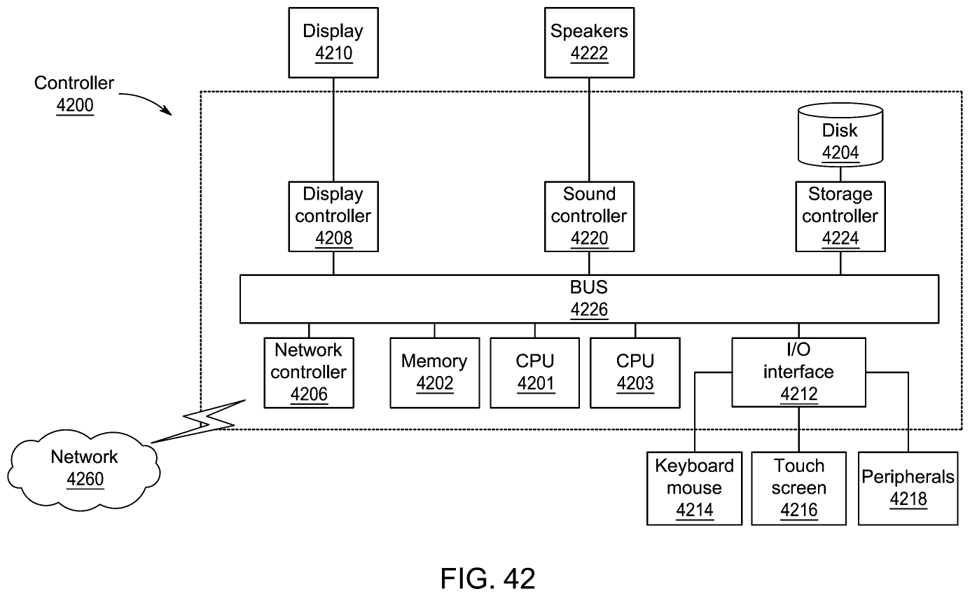

Next, further details of the hardware description of a computing environment according to exemplary embodiments is described with reference to FIG. 42. In FIG. 42, a controller 4200 is described, in which the controller 4200 is a computing device which includes a CPU 4201 which performs the processes described above/below. The process data and instructions may be stored in memory 4202. These processes and instructions may also be stored on a storage medium disk 4204 such as a hard drive (HDD) or portable storage medium or may be stored remotely.

Further, the claims are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the computing device communicates, such as a server or computer.

Further, the claims may be provided as a utility application, background daemon, or component of an operating system, or combination thereof, executing in conjunction with CPU 4201, 4203 and an operating system such as Microsoft Windows 7, Microsoft Windows 8, Microsoft Windows 10, UNIX, Solaris, LINUX, Apple MAC-OS and other systems known to those skilled in the art.

The hardware elements in order to achieve the computing device may be realized by various circuitry elements, known to those skilled in the art. For example, CPU 4201 or CPU 4203 may be a Xenon or Core processor from Intel of America or an Opteron processor from AMD of America, or may be other processor types that would be recognized by one of ordinary skill in the art. Alternatively, the CPU 4201, 4203 may be implemented on an FPGA, ASIC, PLD or using discrete logic circuits, as one of ordinary skill in the art would recognize. Further, CPU 4201, 4203 may be implemented as multiple processors cooperatively working in parallel to perform the instructions of the inventive processes described above.

The computing device in FIG. 42 also includes a network controller 4206, such as an Intel Ethernet PRO network interface card from Intel Corporation of America, for interfacing with network 4260. As can be appreciated, the network 4260 can be a public network, such as the Internet, or a private network such as an LAN or WAN network, or any combination thereof and can also include PSTN or ISDN sub-networks. The network 4260 can also be wired, such as an Ethernet network, or can be wireless such as a cellular network including EDGE, 3G, 4G and 5G wireless cellular systems. The wireless network can also be WiFi, Bluetooth, or any other wireless form of communication that is known.

The computing device further includes a display controller 4208, such as a NVIDIA GeForce GTX or Quadro graphics adaptor from NVIDIA Corporation of America for interfacing with display 4210, such as a Hewlett Packard HPL2445w LCD monitor. A general purpose I/O interface 4212 interfaces with a keyboard and/or mouse 4214 as well as a touch screen panel 4216 on or separate from display 4210. General purpose I/O interface also connects to a variety of peripherals 4218 including printers and scanners, such as an OfficeJet or DeskJet from Hewlett Packard.

A sound controller 4220 is also provided in the computing device such as Sound Blaster X-Fi Titanium from Creative, to interface with speakers/microphone 4222 thereby providing sounds and/or music.

The general purpose storage controller 4224 connects the storage medium disk 4204 with communication bus 4226, which may be an ISA, EISA, VESA, PCI, or similar, for interconnecting all of the components of the computing device. A description of the general features and functionality of the display 4210, keyboard and/or mouse 4214, as well as the display controller 4208, storage controller 4224, network controller 4206, sound controller 4220, and general purpose I/O interface 4212 is omitted herein for brevity as these features are known.

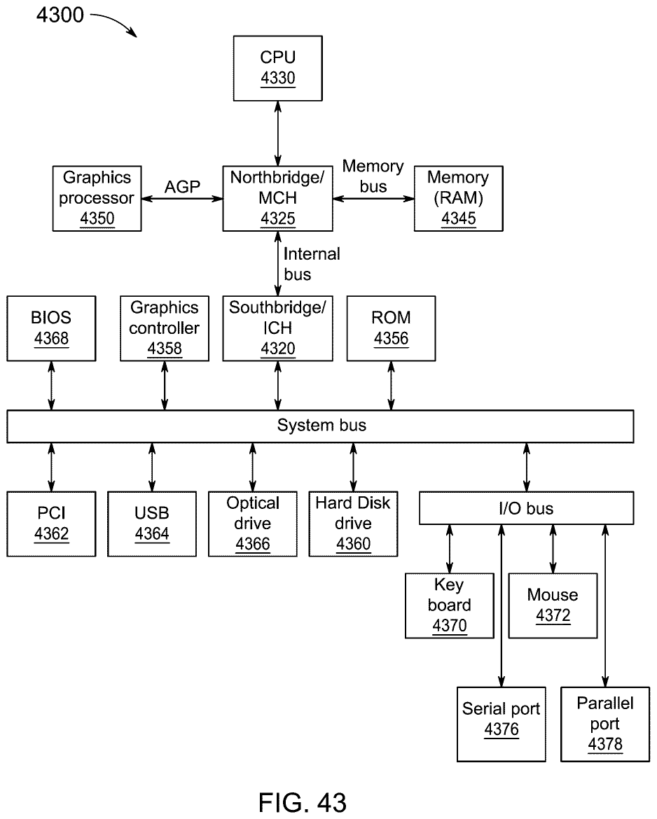

The exemplary circuit elements described in the context of the present disclosure may be replaced with other elements and structured differently than the examples provided herein. Moreover, circuitry configured to perform features described herein may be implemented in multiple circuit units (e.g., chips), or the features may be combined in circuitry on a single chipset, as shown on FIG. 43.

FIG. 43 shows a schematic diagram of a data processing system, according to certain embodiments, for performing the functions of the exemplary embodiments. The data processing system is an example of a computer in which code or instructions implementing the processes of the illustrative embodiments may be located.

In FIG. 43, data processing system 4300 employs a hub architecture including a north bridge and memory controller hub (NB/MCH) 4325 and a south bridge and input/output (I/O) controller hub (SB/ICH) 4320. The central processing unit (CPU) 4330 is connected to NB/MCH 4325. The NB/MCH 4325 also connects to the memory 4345 via a memory bus, and connects to the graphics processor 4350 via an accelerated graphics port (AGP). The NB/MCH 4325 also connects to the SB/ICH 4320 via an internal bus (e.g., a unified media interface or a direct media interface). The CPU Processing unit 4330 may contain one or more processors and even may be implemented using one or more heterogeneous processor systems.

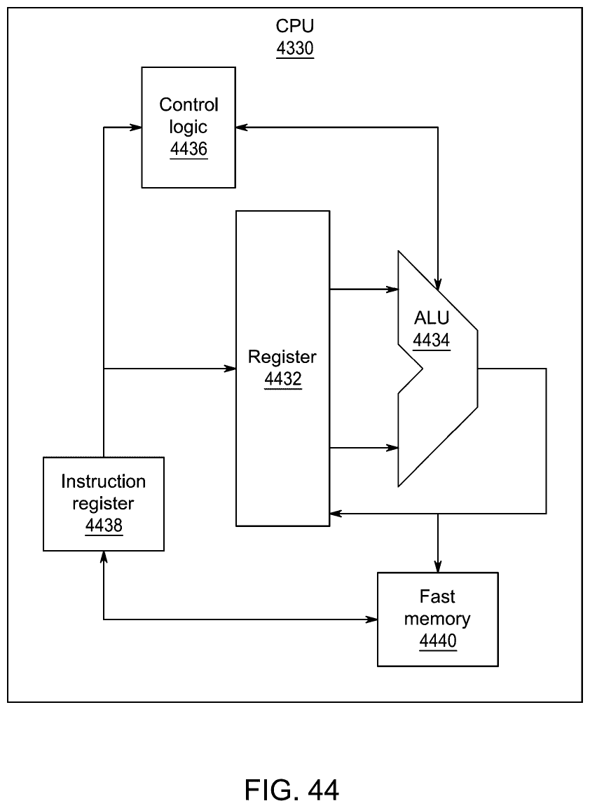

For example, FIG. 44 shows one implementation of CPU 4330. In one implementation, the instruction register 4438 retrieves instructions from the fast memory 4440. At least part of these instructions are fetched from the instruction register 4438 by the control logic 4436 and interpreted according to the instruction set architecture of the CPU 4330. Part of the instructions can also be directed to the register 4432. In one implementation the instructions are decoded according to a hardwired method, and in another implementation the instructions are decoded according a microprogram that translates instructions into sets of CPU configuration signals that are applied sequentially over multiple clock pulses. After fetching and decoding the instructions, the instructions are executed using the arithmetic logic unit (ALU) 4434 that loads values from the register 4432 and performs logical and mathematical operations on the loaded values according to the instructions. The results from these operations can be feedback into the register and/or stored in the fast memory 4440. According to certain implementations, the instruction set architecture of the CPU 4330 can use a reduced instruction set architecture, a complex instruction set architecture, a vector processor architecture, a very large instruction word architecture. Furthermore, the CPU 4330 can be based on the Von Neuman model or the Harvard model. The CPU 4330 can be a digital signal processor, an FPGA, an ASIC, a PLA, a PLD, or a CPLD. Further, the CPU 4330 can be an x86 processor by Intel or by AMD; an ARM processor, a Power architecture processor by, e.g., IBM; a SPARC architecture processor by Sun Microsystems or by Oracle; or other known CPU architecture.

Referring again to FIG. 43, the data processing system 4300 can include that the SB/ICH 4320 is coupled through a system bus to an I/O Bus, a read only memory (ROM) 4356, universal serial bus (USB) port 4364, a flash binary input/output system (BIOS) 4368, and a graphics controller 4358. PCI/PCIe devices can also be coupled to SB/ICH 4388 through a PCI bus 4362.

The PCI devices may include, for example, Ethernet adapters, add-in cards, and PC cards for notebook computers. The Hard disk drive 4360 and CD-ROM 4366 can use, for example, an integrated drive electronics (IDE) or serial advanced technology attachment (SATA) interface. In one implementation the I/O bus can include a super I/O (SIO) device.

Further, the hard disk drive (HDD) 4360 and optical drive 4366 can also be coupled to the SB/ICH 4320 through a system bus. In one implementation, a keyboard 4370, a mouse 4372, a parallel port 4378, and a serial port 4376 can be connected to the system bus through the I/O bus. Other peripherals and devices that can be connected to the SB/ICH 4320 using a mass storage controller such as SATA or PATA, an Ethernet port, an ISA bus, a LPC bridge, SMBus, a DMA controller, and an Audio Codec.

Moreover, the present disclosure is not limited to the specific circuit elements described herein, nor is the present disclosure limited to the specific sizing and classification of these elements. For example, the skilled artisan will appreciate that the circuitry described herein may be adapted based on changes on battery sizing and chemistry or based on the requirements of the intended back-up load to be powered.

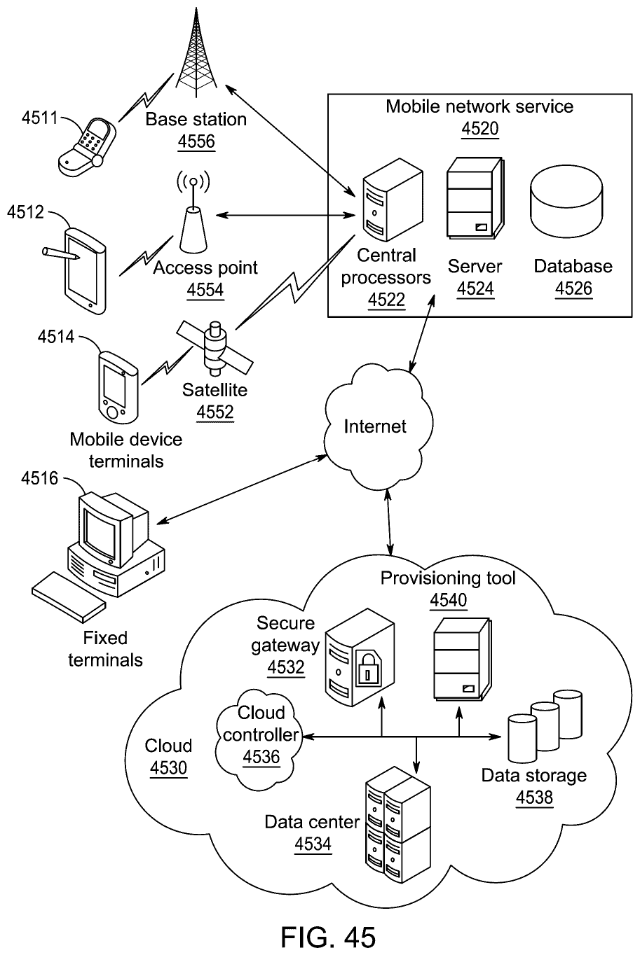

The functions and features described herein may also be executed by various distributed components of a system. For example, one or more processors may execute these system functions, wherein the processors are distributed across multiple components communicating in a network. The distributed components may include one or more client and server machines, such as cloud 4530 including a cloud controller 4536, a secure gateway 4532, a data center 4534, data storage 4538 and a provisioning tool 4540, and mobile network services 4520 including central processors 4522, a server 4524 and a database 4526, which may share processing, as shown by FIG. 45, in addition to various human interface and communication devices (e.g., display monitors 4516, smart phones 4510, tablets 4512, personal digital assistants (PDAs) 4514). The network may be a private network, such as a LAN, satellite 4552 or WAN 4554, or be a public network, may such as the Internet. Input to the system may be received via direct user input and received remotely either in real-time or as a batch process. Additionally, some implementations may be performed on modules or hardware not identical to those described. Accordingly, other implementations are within the scope that may be claimed.

While specific embodiments of the invention have been described, it should be understood that various modifications and alternatives may be implemented without departing from the spirit and scope of the invention. For example, different cellular automata rules or encryption algorithms could be employed, or alternative feature extraction and face recognition techniques could be integrated into the system.

### EXAMPLES

The treatment of critical-sized segmental bone fractures remains a clinical challenge to date. The gold standard treatments for such a condition include the utility of autografts (using a patient's bone), allografts (using a donor's bone), and metallic implants. However, these only available clinical treatments suffer from serious drawbacks and limitations that motivate the investigation of alternative approaches. The focus was on designing, fabricating, and characterizing biodegradable patient-specific three-dimensionally (3D)-printed bone substitutes, plates, and screws. This innovative approach creates a fully biodegradable system customized to each patient's unique anatomy, presenting a promising alternative to conventional treatments.

First, advanced imaging techniques, such as computer tomography (CT) scans, were employed to acquire detailed anatomical data from the patient's defected femoral bones. Segmentation software (3D slicer) and computer-aided design (CAD) software (SolidWorks) were then used to convert these CT scans into 3D models, respectively. This process ultimately resulted in the generation of precise 3D models of the patient's bone defects, enabling us to recreate the volume of the lost bone in three dimensions. Additionally, it facilitated the utilization of the patient's own bone to design custom-fit bone plates and screws tailored to the exact anatomy of their bone. This approach promises superior outcomes by ensuring precise adaptation to the individual patient's needs. After that, 3D printing technologies, specifically fused deposition modeling (FDM), were used to fabricate these 3D models. This process yielded highly porous patient-specific bone substitutes and screws and plates, all made from the biodegradable and biocompatible material polylactic acid (PLA). Lastly, a series of in vitro analyses, including microstructural examinations, mechanical testing, biodegradation assessments, and biocompatibility evaluations, were conducted to assess and affirm the efficacy of the printed system (bone scaffolds, plates, and screws) in terms of their safety and strength.

The results obtained from in vitro experiments were highly promising, highlighting the potential of the designed system in treating critical-sized segmental bone fractures. Microstructural scanning electron microscopy (SEM) analysis revealed that the fabricated scaffolds exhibited a highly porous and intricately interconnected 3D microstructure—a critical feature for improved vascular and cellular infiltration in vivo. Furthermore, SEM imaging highlighted the precision of scaffold printing, as evidenced by the observed variations in pore size among the scaffold groups. Notably, the largest pores were found in scaffolds printed with a 50% pore volume, followed by those with 40% and 30% pore volumes.

Moreover, compression mechanical testing revealed that increasing the pore volume from 30% to 50% led to a decrease in the scaffold's overall mechanical strength. Nevertheless, all scaffold groups demonstrated the capacity to withstand loads significantly surpassing 800 N (the average human weight), affirming their suitability for load bearing applications. Subsequently, torsional mechanical testing was conducted on screws with diameters ranging from 6 to 8 mm. Results indicated that only screws with an 8 mm diameter could withstand the torsional loads typically encountered during placement in the operating room without failure. Conversely, screws with diameters of 6 mm and 7 mm experienced complete failure under similar conditions. As for the plates, they were subjected to a four-point bending mechanical testing. The findings show that increasing the plate thickness from 5 to 7 mm significantly enhances the plates' load-bearing capacity and structural integrity.

The outcomes from the biodegradation test conducted on scaffolds, plates, and screws over two months revealed minimal weight loss %. This outcome is advantageous, as it indicates that they will retain their mechanical strength in the early stages of implantation. Hence, they provide stability and facilitate the functional recovery of the fractured bone during the healing process.

Cell viability assessments using human bone marrow-derived stem cells (hBMSCs) revealed high viability across all scaffold groups at day 7 post-seeding, indicating their excellent biocompatibility. Additionally, cell growth evaluations demonstrated a significant enhancement in hBMSCs' proliferation with increasing scaffold pore volume. Evaluation of cell infiltration revealed successful penetration to the core of scaffolds with 50% and 40% pore volumes, with no obvious differences. However, scaffolds with a 30% pore volume exhibited poor cellular infiltration into their core.

Optimal outcomes were achieved with a patient-specific bone scaffold featuring a 40% pore volume, a 7 mm thick bone plate, and 8 mm diameter bone screws. These findings underscore the potential of this approach, tailored to these specific design parameters, in effectively addressing critical-sized segmental bone fractures. Furthermore, the biodegradable nature of this system offers the added benefit of promoting natural tissue regeneration and eliminating the need for implant removal surgeries, enhancing patient recovery and long-term outcomes.

### Concept Design

First, advanced imaging techniques, such as computer tomography (CT) scans, are employed to acquire detailed anatomical data from the patient's femoral bones. Segmentation software (3D slicer) and computer-aided design (CAD) software (SolidWorks) are used to convert these CT scans into 3D models, respectively. This process ultimately results in the generation of precise 3D models of the patient's bone defects, enabling us to recreate the 3D volume of the lost bone. After that, 3D printing technologies, specifically fused deposition modeling (FDM), are used to fabricate highly porous patient-specific bone substitutes, along with the screws and plates, using the biodegradable and biocompatible polylactic acid (PLA). Lastly, in vitro mechanical testing, biocompatibility evaluations, biodegradation assessments, and microstructural analyses are performed to evaluate and validate the efficacy of the printed bone substitutes, plates, and screws in terms of their safety and strength. The final design of this complete, patient-specific treatment scheme is illustrated in FIGS. 7A-7C.

See also Tafish, N. W, Abdalhaleem, R. T, Albasha, K., Alharbi, M, Alghafli, A. T., Design, Fabrication, and Characterization of Patient-Specific 3D Printed Biodegradable Bone Substitutes, Innovation Exhibition at King Saud University, Riyadh, Saudi Arabia, and Tafish, N. W, Abdalhaleem, R. T, Albasha, K., Alharbi, M, Alghafli, A. T., Design, Fabrication and Characterization of Patient-Specific3D Printed Biodegradable Bone Substitutes [Undergraduate dissertation, Imam Abdulrahman Bin Faisal University], each incorporated herein by reference in its entirety.

### Material Selection

In bone tissue engineering, biocompatibility and degradation rate are crucial factors to consider when designing bone scaffolds, plates, and screws. Since these components will be introduced into the human body, they must be made using a biocompatible material to eliminate immune rejection. Likewise, it is preferable to produce these components using a biodegradable material to eliminate the need for secondary removal surgeries.

In this context, various biocompatible and biodegradable materials are available, including poly lactic acid (PLA), poly glycolic acid (PGA), and poly lactic glycolic acid (PLGA). PLA takes up to 1-1.2 years to completely degrade. On the other hand, PGA and PLGA exhibit a faster degradation rate and completely degrade within 4 weeks and 4 months, respectively. Given the prolonged healing time of bone, which takes up to 1-1.2 years to completely heal and regenerate after critical-sized segmental bone fractures, PLA stands out as a more suitable choice for the application. Consequently, PLA was chosen as the material for the fabrication of the bone scaffolds, plates, and screws.

### Geometrical and Dimensional Considerations Toward Mechanical Competence

In addition to biocompatibility and biodegradation, the mechanical competence and strength achieved in these components is an important factor to consider. This is due to the fact that these components will be placed in load-bearing sites within the bone structure. They will be continuously exposed to mechanical loads, making it crucial for them to withstand these stresses to prevent implant failure after transplantation into the human body.

In this context, various design alternatives were explored for each component, including scaffold pore volume, screw diameter, and plate thickness. The alternatives for each component are thoroughly discussed below.

### Scaffold Pore Volume

Scaffold is the main component in this entire treatment scheme as it will fill the bone defect site, acting as a temporary structure to promote bone formation. The bone scaffolds must display high mechanical strength to provide adequate support during the healing process. Additionally, introducing a high pore volume into the scaffold's structure is essential to enable enhanced cell infiltration, vascularization, and bone ingrowth and regeneration. However, it is important to note that while increasing pore volume is beneficial for tissue regeneration, it may impact the mechanical properties of the scaffold. Therefore, a careful balance between mechanical strength and porosity must be investigated and determined during the design process to ensure optimal scaffold performance. In this sense, various pore volume percentages were considered including, 50%, 40% and 30%, 40% pore volume was selected. This selection arises from the fact that, while bone scaffolds with 50% porosity may display enhanced cellular infiltration, vascularization, and bone regeneration; they may not be able to withstand the harsh mechanical loads being applied in the bony environment. Likewise, bone scaffolds with 30% porosity may indeed display adequate mechanical resilience, but may not promote sufficient cellular infiltration, vascularization, and bone regeneration. Therefore, a bone scaffold with 40% porosity may achieve a balance between mechanical strength and facilitating enhanced bone growth.

### Plate Thickness

Bone plates are medical devices used during surgical procedures to stabilize fractured bones after an injury. When a bone is fractured, bone plates are affixed to the surface of the bone using screws. This helps in preserving the alignment and functionality of the bone, allowing for optimal healing. Therefore, it is extremely important that bone plates exhibit excellent mechanical competence, as the effectiveness of bone fixation and stability relies significantly on their strength.

The currently available bone plates are mainly made from metals such as stainless steel or titanium, due to their biocompatibility and exceptional strength. However, the need for a secondary removal surgery after bone healing is complete is the main limitation of these metallic bone plates. Therefore, one of the aims of this work is to fabricate these bone plates using an entirely biodegradable material, specifically PLA, to ensure their absorption overtime thereby, eliminating the need for additional removal surgeries.

Current metallic bone plates typically have thicknesses ranging between 2-2.5 mm. However, since the plates will be fabricated using PLA, which is mechanically weaker, it is important to consider increasing the overall thickness of the biodegradable plate beyond the standard thicknesses currently used for metallic plates to ensure maintaining the plate's mechanical resilience. Therefore, various thicknesses were considered including, 5 mm, 6 mm, and 7 mm. The biodegradable bone plates were fabricated with a final thickness of 7 mm to ensure the best possible mechanical competence. Thicknesses beyond 7 mm were not considered as these bone plates are meant to be placed on the surface of the bone inside of the body, and thicker plates than 7 mm may not be convenient to the patient. APLA-based bone plate with an overall thickness of 7 mm may provide sufficient strength and fixation.

### Screw Diameter

Bone screws are medical devices used during surgical procedures to secure or fixate bones, plates, or implants to the skeleton structure. Therefore, it is extremely important that bone screws exhibit excellent mechanical competence to withstand mechanical loads.

Bone screws are typically made of biocompatible and strong materials such as stainless steel and titanium. However, like metallic bone plates, these metallic bone screws necessitate secondary removal surgeries after bone healing is achieved. Therefore, one of the aims of this work is to fabricate these bone screws using an entirely biodegradable material, such as PLA, to ensure their absorption overtime thereby, eliminating the need for additional removal surgeries.

Current metallic bone screws typically have diameters ranging between 4-8 mm. However, since the screws were fabricated using PLA, which is mechanically weaker, it is important to consider fabricating these PLA-based screws with the largest possible diameter to ensure maintaining the screw's mechanical resilience. Therefore, various diameters were considered including, 6 mm, 7 mm, and 8 mm. Diameters beyond 8 mm were not considered as screws with a diameter larger than 8 mm may cause soft tissue irritation, bone damage, limited bone healing, and compromised fixation stability. A PLA-based bone screw with an overall diameter of 8 mm may provide sufficient strength and fixation.

### Patient-Specific Bone Scaffold Design

CT scan images of patient's (54 years old, male patient) femoral bones were obtained from Imam Abdulrahman bin Faisal University Hospital after acquiring the patient's consent and proper documentations. These CT scans can then be imported into a segmentation software (3D Slicer, version 5.5.0), where they are converted into 3D models and subsequently exported as STL files (FIG. 8). The 3D femoral bone STL models can then be imported into a computer aided design (CAD) software (SolidWorks) for further processing (FIG. 9). Within SolidWorks, precise measurements have been taken. Initially, two horizontal lines are drawn from the proximal and distal edges of the defect (assuming a defect size of 5 cm) and extended horizontally to the contralateral uninjured bone (FIG. 9). These lines serve as a baseline for recreating the bone defect in the contralateral uninjured femoral bone. By drawing these lines and extending them horizontally to the contralateral uninjured bone, it is possible to identify the segment that aligns anatomically with the defect in the injured bone (FIG. 9). This segment can then be mirrored to match the anatomy of the defect site in the injured bone, resulting in the final 3D model (FIG. 9). This model serves as the basis for creating the patient-specific bone scaffold. Next, pore volumes of 30%, 40%, and 50% were introduced in the scaffold's design within the SolidWorks software (FIGS. 10A-10F).

### Patient-Specific Bone Plate Design

To design the patient-specific bone plates, the patient's bone can be used as a reference to create a bone plate that matches the patient's own anatomy (FIG. 11). These bone plates will morphologically match the patient's geometry (FIG. 11), whereas they will be produced with verifying thicknesses of 5, 6, and 7 mm.

### Patient-Specific Bone Screw Design

To design the bone screws, a standard cortical bone screw design is first used then specific modifications are made. In summary, these adjustments will result in screws with final diameters of 6, 7, and 8 mm (FIGS. 12A-12). Additionally, the screws will be patient-specific, with their length adjusted to match the diameter of the patient's bone for proper fixation and patient convenience (FIG. 13).

### Software Descriptions

To perform the FEA, SolidWorks software was used. SolidWorks is a computer-aided design (CAD) software developed by Dassault Systèmes. It is widely used in engineering and design for creating 3D models, simulations, and technical documentation. SolidWorks offers a range of tools and features that makes it a versatile and powerful solution for various design and engineering applications. One of the tools that SolidWorks offers is FEA of the mechanical properties of 3D models, which will be illustrated in the next section.

### Numerical Modeling

The first step involves applying the desired material to the 3D model from the library of materials found in the software. Given that the 3D models will ultimately be fabricated using PLA, the software was configured with PLA as the designated material for precise analysis. Next, the boundaries are defined, including the constraint surfaces and the areas where physiologically relevant compression forces would be applied to the 3D models. After defining the boundaries, a specific force value, in (newton, N) is applied, and the problem is solved to estimate the mechanical strength of the 3D models.

### Mechanical Analysis

The mechanical test assesses the stress of a part or assembly when subjected to a specific load in compression or torsional modes. This evaluation is crucial for analyzing structural integrity, identifying regions of high stress, and predicting potential failure points.

To perform the mechanical analysis, PLA was first set as the material for each component (FIG. 15). Subsequently, the boundaries were defined, including the constraint surfaces and the areas where physiologically relevant forces would be applied to the 3D models (FIG. 16). The force value was set to 800N, which is equivalent to a body weight of 80 kg. This weight was selected as it represents the average weight of humans. Next, the analysis was run to solve the problem. The stress and deformation data for each component's alternative scaffolds (with pore volumes of 50%, 40%, and 30%), plates (with thicknesses of 5 mm, 6 mm, and 7 mm), and screws (with diameters of 6 mm, 7 mm, and 8 mm) were exported. To mimic the in vivo loading conditions, the scaffolds underwent vertical compression testing, whereas the screws and plate underwent a torsional (torque) and a horizontal 4-points compression testing, respectively.

### Scaffolds Compressive Analysis Results

FIGS. 17A-17C show the strain data for the different scaffolds. As expected, increasing the pore volume in the scaffold from 30% to 50% resulted in decreasing the scaffold's overall mechanical strength. Specifically, the stress values for the scaffolds with pore volumes of 30%, 40%, and 50% was found to be 2.79 MPa, 9.7 MPa, and 33.31 MPa, respectively. The observed increase in stress values with increasing the pore volume suggests a decrease in the scaffold's overall mechanical strength. Higher stress values correspond to weaker scaffolds.

Nevertheless, all scaffolds demonstrated the ability to withstand the applied load of 800N without failure, indicating their suability for the intended load-bearing application. This is evident from the fact that all scaffolds exhibited stress values lower than their maximum yield value of 127 MPa (maximum stress value they can withstand before failing).

### Plates 4-Point Bending Compressive Analysis Results

FIGS. 18A-18C show the strain data for the different plates. As anticipated, increasing the thickness of the plate from 5 mm to 7 mm resulted in increasing the plate's overall mechanical strength. Specifically, the stress values for the plates with thicknesses of 5 mm, 6 mm, and 7 mm was found to be 46.35 MPa, 7.43 MPa, and 7.11 MPa, respectively. The observed increase in stress values with decreasing the plate's thickness suggests a decrease in the plate's overall mechanical strength. Higher stress values correspond to weaker plates.

Nevertheless, all plates demonstrated the ability to withstand the applied load of 800N without failure, indicating their suability for the intended load-bearing application. This is evident from the fact that all plates exhibited stress values lower than their maximum yield value of 127 MPa (maximum stress value they can withstand before failing).

### Screws Torsional Analysis Results

FIGS. 19A-19C show the strain data for the different screws. As predicted, increasing the screw's diameter from 6 mm to 8 mm resulted in increasing the screw's overall mechanical strength. Specifically, the stress values for the screws with diameters of 6 mm, 7 mm, and 8 mm was found to be 239 MPa, 142 MPa, and 98.03 MPa, respectively. This indicates that as the screw's overall diameter was increased, its mechanical strength increased.

However, the data revealed that only screw with a thickness of 8 mm could withstand the applied load without failure, whereas the screws having diameters of 6 mm and 7 mm experienced complete failure. This is evident by the fact the screw with a thickness of 8 mm had a stress value of 98.03 MPa, which is lower than its yield strength value of 127 MPa. On the other hand, the stress values for the screws with thicknesses of 6 mm and 7 mm were 239 MPa and 142.2 MPa, respectively. These values exceeded the screw's yield strength, indicating failure.

### Detailed Description of the Final Design

The goal was to design and develop patient-specific, biodegradable, and mechanically robust 3D-printed bone substitutes, screws, and plates tailored for critical-sized segmental bone defects. The design of a comprehensive approach for producing anatomically matched and patient-specific 3D models of bone substitutes, plates, and screws was demonstrated. This advancement contributes to an improved treatment paradigm for critical-sized segmental bone fractures.

The use of biodegradable materials, specifically PLA, in fabricating these 3D models of scaffolds, plates, and screws was an advantageous approach. One of these advantages is that the components that will be absorbed overtime to eliminate the need for additional removal surgeries and accelerate the overall healing process.

Since these components are intended to be utilized in load-bearing anatomical sites, they must be designed with robust mechanical properties. Consequently, the scaffolds, plates, and screws were initially designed with varying pore volumes, thicknesses, and diameters, respectively. Computational analysis of their mechanical competence and robustness was then conducted to finalized the optimal design for each component. Based on the FEA conducted above, all scaffolds demonstrated the ability to withstand the applied mechanical load without failure, indicating their suitability.

However, considering previous literature suggesting that scaffolds with a pore volume of 30% may not promote sufficient cellular infiltration, vascularization, and bone regeneration, and recognizing the potential failure in vivo with scaffolds exhibiting 50% pore volume, the final scaffold design used a 40% pore volume. This decision aims to achieve a balance by maintaining mechanical strength while offering an adequate pore volume for the intended purposes.

Further FEA revealed that a bone plate and screw with a final thickness of 7 mm and a diameter of 8 mm, respectively, exhibited the best resistance to the applied mechanical load without failure, confirming their suitability. Therefore, the final design for the plates and screws would feature a thickness of 7 mm and a diameter of 8 mm, respectively.

### Cost Analysis

The table below outlines the cost of materials utilized in the design and fabrication of scaffolds, screws, and plates.

TABLE 19
A list of the Materials utilized in the design and fabrication
of scaffolds, screws, and plates and their associated cost.

| | Amount/volume/ | |
|---|---|---|
| Item Name | QTY | Total Price (SAR) |
| CT scans | NA | Provided by university |
| | | hospital |
| SolidWorks software | NA | Provided by IAU |
| 3D Slicer software | NA | Free |
| 3D slicing software | NA | Free |
| FDM 3D printer | NA | Provided by IAU |
| PLA filament | 2 kg | 200 |

*NA indicated (Not Applicable)

As shown in table 19, it is evident that the approach is cost-effective, as the only expense to be considered is the cost of the polymer. For more accurate cost analysis, the weight of the scaffold, plate, and screws intended for a single patient were measured, which allowed more precise estimation the total cost for all components based on their weight. Furthermore, while the PLA is biocompatible and biodegradable, it is not medical-grade, and therefore, it is cost-effective. Medical grade PLA costs around 26.25 SAR per gram. In the cost analysis, the price of the medical grade PLA was used for a more accurate reflection of real-world costs. Yet, a cost analysis using the PLA used was also included for documentation purposes. The table below outlines the weight of each component and its corresponding cost:

TABLE 20
Cost analysis of the technology/ patient.

| | | Price/ | Price/ gram | | | Total |
|---|---|---|---|---|---|---|
| | | gram | (non- | | Total price | price (non- |
| | | (Medical | medical | | (medical | medical |
| | Weight | Grade) | Grade) | QTY | grade) | grade) |
| Component | (g) | (SAR) | (SAR) | needed | (SAR) | (SAR) |
| Scaffold | 19 | 26.25 | 0.1 | 1 | 498.75 | 1.9 |
| Plate | 204 | 26.25 | 0.1 | 1 | 5355 | 20.4 |
| Screw | 2.53 | 26.25 | 0.1 | 5 | 332.06 | 1.26 |
| | | | | Total | 6185.81 | 23.56 |
| | | | | net price | | |

As shown in table 20, the total cost for implementing the technology, which includes a complete set of biodegradable scaffolds, plates, and screws, is estimated to cost 6185.81 SAR and 23.56 SAR for the medical and non-medical grade PLA, respectively, per patient. Compared to current technologies, including allografts, autografts, and metallic implants, and the associated metallic plates and screws often used with them, the technology represents a more cost-effective alternative as demonstrated in table 21.

TABLE 21
Cost analysis of current technologies/patient.

| Component | Price (SAR) | QTY needed | Total price (SAR) |
|---|---|---|---|
| Autograft | 9,375-13125 | Patient-specific | 9,375-13125 (Price |
| | | | of harvesting) |
| Allograft | 3312.49 | Patient-specific | 3312.49 |
| Metallic implant | 6088.50 | Patient-specific | 6,088.50 |
| Metallic plate | 2500-5000 | 1 | 2500-5000 |
| Metallic screw | 500-1500 | 5 | 2500-7500 |
| | | Total net price for | 14,375-25,625 |
| | | autograft-based | |
| | | treatment | |
| | | Total net price for | 19481.9625 |
| | | allograft-based | |
| | | treatment | |
| | | Total net price for | 11,088.5-18,588.5 |
| | | metallic implant- | |
| | | based treatment | |

The cost of hospitalization, procedure, and rehabilitation were excluded from the cost analysis, since the focus here was to estimate the cost of the developed technology compared to other currently available alternatives. However, the overall cost for these elements would likely decrease if this technology was to be implemented. This reduction is attributed to the fact that secondary removal surgeries are eliminated, given that biodegradable materials that will degrade overtime are used. Thus, any costs associated with hospitalization, procedure, and rehabilitation for the second removal surgeries are effectively eliminated.

### Experimental Tests

A series of in vitro experiments were conducted to assess and confirm the accuracy, safety, and efficacy of the system, including microstructural scanning electron microscopy (SEM) analysis, mechanical testing, biodegradation assessments, and biocompatibility evaluations.

### Patient-Specific Bone Scaffold, Plate, and Screws Design

To design the patient-specific bone scaffolds, computer tomography (CT) scans of patients' femoral bones were obtained from Imam Abdulrahman bin Faisal University Hospital after acquiring the patients' consent and proper documentations. These CT scans were imported into a segmentation software (3D Slicer, version 5.5.0), where they were converted into 3D models and subsequently exported as STL files. The 3D femoral bone STL models were then imported into a computer aided design (CAD) software (SolidWorks) for further processing. Within SolidWorks, precise measurements were taken. Initially, the distance from the hip joint down to the proximal edge of the defect was recorded. Subsequently, the distance from the proximal edge of the defect to the distal edge was also measured and documented. These measurements served as a baseline for recreating the bone defect in the contralateral uninjured femoral bone. By applying the same measurements to the uninjured contralateral femoral bone, the segment that aligns anatomically with the defect in the injured bone was identified. This segment was mirrored to match the anatomy of the defect site in the injured bone, resulting in the final 3D model. This model served as the basis for creating the patient-specific bone scaffold. Next, the generated model was imported into a slicing software (PrusaSlicer version 2.7.1) for further refinement. Within the PrusaSlicer, infill percentages of 50%, 60%, and 70%, yielding 3D bone scaffolds with corresponding porosities of 50%, 40%, and 30% were implemented.

To design patient-specific bone plates, the 3D model of the patient's femoral bone was imported into SolidWorks software. The Power Surfacing feature was employed to design a bone plate tailored to the specific anatomy of the lateral part of the patient's femoral bone, yielding a patient-specific bone plate. Subsequently, the same bone plate was replicated with varying final thicknesses of 5, 6, and 7 mm.

To design patient-specific bone screws, the diameter of the patient's femoral bone 3D model was measured at four separate locations (two distally and two proximally). These diameters were then utilized to determine the length of the screws, ensuring that they are tailored to the patient's specific bone dimensions for enhanced convenience. Subsequently, the same screws were replicated with different final diameters of 6, 7, and 8 mm.

### Patient-Specific Bone Scaffold, Plate, and Screws Fabrication

The patient-specific bone scaffolds, along with the screws and plates, were manufactured using biodegradable poly lactic acid (PLA) through 3D printing technology, using a fused deposition modeling (FDM) technique. A 3D printer (Prusa TRILAB AzteQ Industrial) was used to fabricate the scaffolds, while the (MakerBot Replicator Z18) was used to fabricate the screws and plates (FIGS. 20A-20B).

### In Vitro Experiments

### Scanning Electron Microscopy (SEM)

To assess the microstructure of the scaffolds, they were mounted on 15 mm stubs, gold sputter-coated using a sputter cotter machine for 3 minutes to eliminate surface charging and imaged using an SEM machine at a working distance of 5 mm and an acceleration voltage of 18 kV (FIGS. 21A-21D). The SEM images were used to measure the pore size in each scaffold group using the ImageJ software (version 1.4 g, National Institute of Health, USA).

### Mechanical Testing

To assess the mechanical strength of the scaffolds, they were placed perpendicularly between two metallic plates of a mechanical tester machine (Instron) and compression load was applied onto the scaffold until a complete scaffold failure is achieved (N=6/group). The generated force (in newtons) and displacement (in mm) data were used to calculate the compressive strength and compressive modulus. To assess the mechanical strength of the bone plates, a 4-point bending mechanical test was conducted. Briefly, the bone plates were placed horizontally onto two supporting beams spaced at a distance of 40 mm apart within the Instron mechanical tester machine (N=6/group). Subsequently, two loading piece spaced at a distance of 20 mm was descend towards the sample until contact is made, and the test proceeded until failure occurs. The generated force (in newtons) and displacement (in mm) data was used to calculate the compressive strength and compressive modulus.

To assess the mechanical strength of the bone screws, a torsion test was performed using a mechanical tester (Instron machine) (N=6/group). Briefly, the screws were positioned between two grips (one fixed and one rotating grip), and each end of the screws was attached to the grips. Next, a torsion force was applied from one end until a complete failure was achieved. The threshold torque (TT), peak failure torque (PFT), and peak clamping torque (PCT) values were measured and recorded for each sample. FIGS. 22A-22C illustrate the setup of the mechanical tests that were performed on the fabricated bone scaffolds, plates, and screws.

### Biodegradation Testing

To assess the biodegradation of the bone scaffolds, plates, and screws, their initial weights (Wi) was recorded. The samples were then transferred into 50-mL tubes containing 50 mL of water and maintained at 37° C. under gentle agitation (FIGS. 23A-23C). The water was changed every one week. After two months, the samples were harvested, frozen, freeze dried, then weighed (Wf). The biodegradation of the samples (N=3/group) was calculated using the following formula:

$$Biodegradation \% = \frac{W i - W f}{W i} \times 100 \qquad Equation ( 1 )$$

Where (Wi) is the initial weight, and (Wf) is the final weight.

### Bone Marrow-Derived Stem Cells Culture

Human Bone Marrow-derived stem cells (BMSCs) were used to assess the biocompatibility of the resultant scaffolds. Briefly, BMSCs were cultured in growth media (DMEM: F12, Gibco, USA) supplemented with 1% penicillin and streptomycin (P/S, Gibco) and 10% fetal bovine serum (FBS, Gibco) in T-182 flasks and maintained in a humidified tissue culture incubator at 37° C. and 5% CO₂. Cells at the 4^{th}-6^{th} passage were used for all experiments. Media was changed every 2 days for all experiments.

### Biocompatibility Evaluations

The objective of this procedure is to assess the biocompatibility of the resulting scaffolds, evaluating their capability to support cell viability and growth within their structures. To assess the biocompatibility of the scaffolds, they were first sterilized. For sterilization, the scaffolds were submerged in 70% ethanol for 30 minutes, washed twice with PBS, then exposed to ultra-violet (UV) light for 30 minutes each side. Next, BMSCs were seeded onto 5 mm×5 mm scaffolds at a final density of 100k cells/scaffold. The seeded scaffolds were then transferred into 96-well plates, and 200 μL of growth media was added to each well. Cell viability was assessed using LIVE/DEAD cytotoxicity assay kit (Invitrogen, USA) at day 7 of culture. Briefly, at day 7, the scaffolds were transferred to new wells, rinsed twice with phosphate buffered saline (PBS Gibco), and then incubated with 100 μL of the kit's staining solution (100 uL PBS, 0.05 uL calcein AM, and 0.2 uL ethidium homodimer-1) for 15 minutes at room temperature. Afterward, the samples were washed twice with PBS and imaged in PBS using an inverted fluorescence microscope. Cell growth was assessed and quantified (N=3/group) using the CellTiter 96® AQueous One Solution Cell Proliferation Assay (MTS, Promega Inc, USA) at day 7 of culture. Briefly, at day 7, the samples were transferred to new wells, washed twice with PBS, and then incubated with 200 μL of the MTS solution (40 uL MTS solution, 160 uL growth media) for 2 hours at 37° C. Next, 100 μL from the supernatant of each sample was transferred to 96-well plates, and the absorbance was read at 490 nm using a plate reader. To eliminate the interface of MTS reagent with the samples, acellular scaffolds were incubated with the MTS reagent, and their absorbance values were deducted from the absorbance values measured for each cellular scaffold. FIG. 24 illustrates the process employed for assessing cell viability and growth.

All quantitative data were expressed as mean±standard deviation. All statistical analyses were performed using the statistical software Prism GraphPad version 8 (GraphPad, USA). Statistical analyses were performed using the one-way analysis of variance (one-way ANOVA) with Tukey's post hoc test. Statistical significance was evaluated at *p<0.05. **p<0.01, ***p<0.001, and ****p<0.0001.

### Scaffolds Characterizations

The outcomes of the in vitro experiments conducted to characterize the fabricated scaffolds with 30%, 40%, and 50% pore volumes are presented below. The analysis encompasses scanning electron microscopy, mechanical testing, biodegradation assessment, and biocompatibility evaluations (cell growth and viability).

### Scanning Electron Microscopy (SEM) Analysis

Scanning electron microscopy (SEM) analysis was employed to examine the microstructure and variation in pore sizes within the fabricated scaffolds. FIGS. 25A-25F shows the SEM images of the 30%, 40%, and 50% scaffolds captured at 50× and 100× magnifications. These scaffolds were fabricated using 3D printing techniques, specifically fused deposition modeling (FDM). The SEM images reveal that these scaffolds maintain a highly porous microstructure and 3D interconnectivity—a crucial feature to ensure facilitating nutrient and oxygen delivery, waste exchange, cell infiltration, and vascular ingrowth throughout the scaffold in vivo.

Qualitatively, it was evident that the pore size in the 50% pore volume scaffold group was the largest, followed by the 40% pore volume scaffold, with the 30% pore volume scaffold group exhibiting the smallest pores. These qualitative findings were further confirmed quantitatively by measuring the pore size in each scaffold group using the ImageJ software (See appendix A for detailed data). Quantitative pore size analysis revealed that the pore size was the highest in the 50% pore volume scaffold group, followed by the 40% and 30% pore volume scaffold groups FIG. 25G. Statistically, the pore size was significantly higher in the 50% and 40% pore volume scaffold groups compared to the 30% pore volume scaffold group. However, no statistically significant differences were observed between the 50% and 40% pore volume scaffold groups. Overall, the observed variations in pore size among the scaffold groups is an indication of the printing accuracy.

Indeed, bone tissue engineering scaffolds must display high mechanical strength to provide adequate support during the healing process. Additionally, introducing a high pore volume into the scaffold's structure is essential to enable enhanced cell infiltration, vascularization, and bone ingrowth and regeneration. However, it is important to note that while increasing pore volume is beneficial for tissue regeneration, it may impact the mechanical properties of the scaffold. Therefore, a careful balance between mechanical strength and porosity must be investigated and determined during the design process to ensure optimal scaffold performance. While bone scaffolds with 50% porosity may display enhanced cellular infiltration, vascularization, and bone regeneration; they may not be able to withstand the harsh mechanical loads being applied in the bony environment. Likewise, bone scaffolds with 30% porosity may indeed display adequate mechanical resilience, but may not promote sufficient cellular infiltration, vascularization, and bone regeneration as shown in previous research. Therefore, a bone scaffold with 40% porosity may achieve a balance between mechanical strength and facilitating enhanced cellular infiltration. Yet, further in vitro mechanical validation will be required to confirm this hypothesis, which will be achieved in the following section.

### Compression Mechanical Testing

Compression mechanical testing was conducted on the various scaffold groups to assess their mechanical strength, given their intended placement in a load-bearing anatomical site. FIGS. 26A-26B presents the recorded values of compressive modulus and compressive strength for the 30%, 40%, and 50% pore volume scaffolds following the application of compression force, accompanied by the performed statistical analysis.

Compressive stress refers to the maximum load an object can withstand before initiating breakage, while compressive modulus indicates the maximum load the object can withstand before complete failure. Results indicate that increasing the scaffold's pore volume from 30% to 50% led to a reduction in overall mechanical strength. Specifically, the average compressive modulus values for scaffolds with pore volumes of 30%, 40%, and 50% were 903.28 MPa, 790.27 MPa, and 560.2651 MPa, respectively. Additionally, the average compressive strength values for the same groups were 48.48304 MPa, 40.09623 MPa, and 32.401432 MPa, respectively. These values fall within the same range as those reported for human bone, indicating their suitability for application in boney load bearing applications.

These findings are in line with numerical values acquired from finite element analysis (FEA) conducted earlier, which demonstrated a decrease in scaffold's overall mechanical strength with increasing pore volume. FIGS. 26A-26B present the statistical analysis of compressive modulus and compressive strength of the different scaffold groups, respectively. Significant differences were observed among all scaffold groups, indicating a substantial enhancement in scaffold capability to withstand mechanical forces and maintain structural integrity as pore volume decreases. This improvement in mechanical properties with decreasing pore volume is attributed to the fact that as the pore volume decreases, more material occupies the scaffold volume and less empty space, enabling the scaffold to tolerate greater compression forces.

Regardless of the variation in strength among the different scaffold groups, all scaffolds demonstrated the ability to withstand loads significantly exceeding 800 N (average human weight) without failure (See appendix A for detailed data). Overall, these data indicate the suitability of all scaffold groups for the intended load-bearing applications.

### Biodegradation Assessment

The degradation test primarily aims to assess the rate of biodegradation of implants, facilitating the evaluation and selection of biomaterials for bone scaffolds. Ideally, these materials should exhibit slow degradation rates during the early healing stages (first 2-4 months) to maintain their mechanical strength and structural integrity and degrade in proportion to the rate of new bone formation.

In this study, the biodegradation rate of all scaffold groups under physiological conditions was investigated (37° C. and gentle agitation) to determine if scaffold pore volume affects the biodegradation rate. This involved measuring the initial and final weights of the samples and calculating the weight loss percentage using Equation 1 (See appendix A for detailed data). The results revealed that after two months, the weight loss percentage for all scaffold groups was approximately 0.54%, demonstrating the suitability of the selected material for the intended application (FIG. 27). Furthermore, no statistically significant differences were observed between the groups, suggesting that pore volume does not influence the scaffold biodegradation rate.

Within just two months of implanting the scaffolds, regardless of pore volume, the degradation rate remained relatively stable since they were composed of the same material. Moreover, the observed degradation rate over this period is advantageous, as substantial bone growth has not yet occurred. Significant weight loss from the scaffolds during this timeframe could compromise their mechanical strength, potentially leading to implant failure.

Typically, the degradation rate of PLA-fabricated bone scaffolds during the initial six months is relatively slow. However, as the scaffolds approach their breakdown point, the biodegradation process accelerates due to the increased ease of breaking bonds and polymers. By this time, a reliable amount of bone should have grown, reinforcing the mechanical integrity of the defect site along with the scaffold remnants until complete bone formation occurs.

### Biocompatibility Evaluations

Scaffold biocompatibility is an important consideration in bone tissue engineering, predominantly influenced by material selection and scaffold design. In this study, PLA was used as the primary material across all scaffold groups due to its established biocompatibility. However, the key difference among these groups was the variance in scaffold pore volume. Generally, scaffolds with larger pore volumes support enhanced cell growth and infiltration into their cores, whereas those with smaller pore volumes often exhibit limited cell infiltration and growth. The investigation involved fabricating scaffolds with varying pore volumes of 30%, 40%, and 50%, aiming to examine the impact of pore volume in the scaffold design on cell viability, infiltration, and growth. For biological evaluations, human Bone marrow-derived stem cells (hBMSCs) were used due to their relevance and widespread use in bone tissue engineering research, given their role in bone tissue formation.

Cell viability and growth assessments were conducted by culturing hBMSCs on all scaffold groups for 7 days. The viability assessment primarily focused on assessing the infiltration of cells into the scaffold cores using the live/dead assay. Notably, greater cell infiltration was observed in the 40% and 50% scaffold groups compared to the 30% group (FIG. 28A-28C). In the 30% scaffold group, minimal cell presence was noted at the core, accompanied by some dead cells, likely attributed to inadequate oxygen and nutrient delivery due to smaller pore size, as indicated by SEM analysis (FIGS. 25A-25G). Conversely, by day 7, cells had successfully infiltrated the cores of the 40% and 50% scaffold groups, maintaining viability possibly due to larger pore sizes facilitating sufficient oxygen and nutrient delivery, as indicated by SEM analysis. These results also further validate the biocompatibility of the chosen material.

In addition to viability evaluations, growth analysis using an MTS assay revealed cell growth was the highest in the 50% group, followed by the 40% and then the 30% groups (FIG. 28D) (See appendix A for detailed data). Statistically, the cell growth was significantly higher in the 50% scaffold groups compared to the 30% scaffold group. However, no statistically significant difference was observed between the 40% and 50% groups, indicating similar support for cell growth.

While the 50% scaffold group demonstrated the highest cell growth, the lack of significant difference between it and the 40% scaffold group suggests that the 40% scaffold group may be more suitable for load-bearing bone tissue engineering applications. This preference stems from the 40% group exhibiting significantly higher mechanical strength compared to the 50% group, highlighting its potential superiority in supporting the demands of load-bearing scenarios.

### Bone Plates Characterizations

The outcomes of the in vitro experiments conducted to characterize the fabricated plates with 5 mm, 6 mm, and 7 mm thicknesses are presented below. The analysis encompasses mechanical testing and biodegradation assessment.

### Four-Points Bending Mechanical Testing

The mechanical analysis of bone plates was conducted using the four-point bending method, a standard approach mandatory by ASTM F382 for assessing the mechanical properties of bone plates. This method involves subjecting the specimen to bending forces at two distinct points, creating a central region where deformation occurs while maintaining support at both ends (FIG. 22B). This setup allows for a more controlled assessment of mechanical behavior compared to other bending tests, making it particularly suitable for evaluating the strength and stiffness of materials, including bone plates.

Following the four-point bending mechanical test, data on compressive strength and compressive modulus were recorded for plates with varying thicknesses of 5 mm, 6 mm, and 7 mm. Statistical analysis was then performed to interpret the results.

In terms of compressive strength at maximum load, thicker plates exhibited higher mean values. Specifically, the 7 mm plate thickness showed the highest mean compressive strength at a maximum load of 85.39 MPa, followed by the 6 mm plate thickness at 70.19 MPa, with the 5 mm plate thickness displaying the lowest mean value of 52.89 MPa. This trend suggests that increasing plate thickness enhances the plates' ability to resist deformation and failure under compressive forces.

Similarly, the compressive modulus demonstrated a parallel trend, with thicker plates showing higher mean values, indicating increased stiffness and resistance to elastic deformation. The 7 mm plate thickness recorded the highest mean compressive modulus of 2577.97 MPa, followed by the 6 mm plate thickness at 1941.85 MPa, and the 5 mm plate thickness at the lowest mean value of 1331.08 MPa. These results imply that augmenting plate thickness enhances their load-bearing capacity and structural integrity.

FIGS. 29A-29B illustrate the statistical analysis of compressive stress and compressive modulus for different plate groups, respectively, revealing significant differences between all plate groups (5 mm vs. 6 mm, 6 mm vs. 7 mm, and 5 mm vs. 7 mm thickness). This highlights the importance of increasing these parameters to strengthen plates' ability to withstand mechanical forces and loads.

Augmenting bone plate thickness positively impacts their mechanical strength. Thicker plates exhibit higher compressive stress and compressive modulus (See appendix C for detailed data), suggesting improved resistance to deformation and increased load-bearing capacity. Additionally, these findings are in line with numerical values acquired from finite element analysis (FEA) conducted earlier in section 3.4, which demonstrated an increase in plate's overall mechanical strength with increasing the plate thickness.

### Biodegradation Assessment

The biodegradation test was conducted on the plate samples following the same protocol as the scaffold assessment. The results shown in FIG. 30 indicate that the average degradation rates for plates with thicknesses of 5 mm, 6 mm, and 7 mm are 0.45%, 0.47%, and 0.43%, respectively. Notably, there were no statistically significant differences observed among the plate groups, as illustrated in FIG. 30.

These findings indicate that the bone plate maintains its mechanical integrity during the initial stages of implantation—a crucial requirement for ensuring successful fracture healing, stability, and functional recovery of the affected bone during the critical healing period. They also indicate that the plate thickness does not influence the plate biodegradation rate (See appendix A for detailed data).

### Bone Screw Characterization

The outcomes of the in vitro experiments conducted to characterize the fabricated screws with 6 mm, 7 mm, and 8 mm diameters are presented below. The analysis encompasses mechanical testing and biodegradation assessment.

### Torsional Mechanical Testing

The torsional mechanical analysis of bone screws, as mandated by ASTM F543, provides a comprehensive assessment of their ability to withstand torsion loads, crucial in orthopedic applications. This method was employed to evaluate the mechanical properties of the designed bone screws, utilizing different diameters (6 mm, 7 mm, and 8 mm).

During the analysis, values of threshold torque (TT), peak failure torque (PFT), and peak clamping torque (PCT) were measured and recorded, offering significant insights into the screws' mechanical behavior (FIGS. 31A-31C). TT represents the minimum torque required for proper fixation and stability of the bone screw, while PFT is the maximum torque a screw can withstand under torsion before it starts failing. Typically, the PFT value must surpass the TT value to prevent failure.

In the torsion test results (FIG. 31A), TT values were determined to be 368.6224 N·mm, 368.4719 N·mm, and 377.3779 N·mm for 6 mm, 7 mm, and 8 mm diameter screws, respectively. Although no statistically significant difference was observed between screw groups, it's essential to note the relevance of TT in ensuring screw stability.

Moving to PFT values (FIG. 31B), screws with diameters of 6 mm, 7 mm, and 8 mm exhibited PFT values of 338.9238 N·mm, 359.6666 N·mm, and 570.6356 N·mm, respectively. Significant differences were noted between 6 mm and 8 mm screws, and between 7 mm and 8 mm screws. Only the 8 mm screw could withstand applied torsion loads without failure, evidenced by its PFT exceeding the TT value.

Earlier finite element analysis (FEA) simulations corroborated these findings, indicating that only 8 mm diameter screws could resist torsion without failure. The experimental results aligned well with the FEA predictions.

Furthermore, peak clamping torque (PCT) values (FIG. 31C) were assessed to determine the maximum torque required for optimal compression between bone and screws, essential for promoting stability and healing. Screws with 6 mm, 7 mm, and 8 mm diameters recorded PCT values of 353.7731 N·mm, 381.9641 N·mm, and 474.0068 N·mm, respectively. Again, the 8 mm screw achieved optimal compression without failure, supported by its PCT value being lower than the PFT value.

The torsional mechanical analysis provided valuable insights into the performance of bone screws. Only the 8 mm diameter screw demonstrated sufficient mechanical strength to withstand torsion loads without failure, consistent with FEA predictions (See appendix A for detailed data). These findings highlight the importance of selecting appropriate screw dimensions to ensure optimal stability and fracture healing in orthopedic applications.

### Biodegradation Assessment

Ensuring the durability and stability of screws, coupled with controlled degradation, is crucial for maintaining stable fixation and load-bearing capacity during the critical initial 6-month period post-implantation. Minimizing degradation rates is paramount to sustain the screws' support and structural integrity, vital for successful healing and sustained implant effectiveness.

In FIG. 32, the results reveal that over a two-month period, a 6 mm diameter screw experienced a weight loss of 0.36%, a 7 mm diameter screw saw a loss of 0.53%, and an 8 mm diameter screw exhibited a loss of 0.45%. These findings are promising, indicating that the screws maintained their mechanical strength and structural integrity during this early duration, ensuring proper fixation and stabilization of the bone plate (See appendix A for detailed data).

Statistical analysis conducted on the 6 mm, 7 mm, and 8 mm diameter bone screws reveals notable variations in the evaluated parameter (weight loss), as illustrated in FIG. 32. These variations suggest potential changes in performance or efficacy associated with the diverse measurements, highlighting the need for further investigation into their implications on long-term implant stability and efficacy.

Furthermore, the biodegradation data indicate that the screw diameter does not significantly influence its biodegradation rate, as all screws displayed low degradation rates over the tested duration.

### Complete System

A final prototype of this complete, patient-specific treatment scheme is illustrated in FIGS. 34-41 and is composed of the patient's fractured bone along with the fabricated patient-specific 40% pore volume bone scaffold, 7 mm thick bone plate, and 8 mm diameter bone screws. This prototype was fabricated using biodegradable polylactic acid (PLA) through 3D printing technology, using a fused deposition modeling (FDM) technique, ultimately resulting in a patient-specific and completely biodegradable system for critical size segmental bone fractures treatment.

The patient-specific 40% pore volume fabricated bone scaffold is shown in FIG. 35. This scaffold serves as a framework to support the formation of functional bone tissue by maintaining mechanical strength while offering an adequate pore volume for promoting sufficient cellular infiltration, vascularization, and bone regeneration.

The patient-specific 7 mm thick fabricated bone plate is illustrated in FIG. 36. This plate is used during surgical procedures to stabilize fractured bones after an injury, preserving the alignment and functionality of the bone and allowing for optimal healing.

The patient-specific 8 mm diameter fabricated bone screws are shown in FIG. 37. These screws are used during surgical procedures to secure or fixate bone plates to the skeleton structure. Additionally, the length of screws was adjusted to match the diameter of the patient's bone to prevent bone tissue irritation and ensure mechanical stability (see FIG. 36).

Addressing critical-sized segmental bone fractures remains a persistent challenge in clinical practice, prompting exploration beyond conventional treatments. A promising discipline for the treatment of bone fractures is regenerative engineering, which utilizes the most cutting-edge technologies seen in various fields, such as material science and engineering, to produce favorable adaption technologies for injured or lost tissue regeneration. This work focused on the development of patient-specific, biodegradable 3D-printed bone scaffolds, plates, and screws, aiming to overcome limitations of the existing methods. Through thorough design, fabrication, and in vitro analyses, this study demonstrated the potential of these customized and completely biodegradable constructs in reshaping fracture treatment paradigms.

The findings underscored the efficacy of a patient-specific bone scaffold featuring a 40% pore volume, a 7 mm thick bone plate, and 8 mm diameter bone screws. These optimized parameters align with structural, mechanical, and degradative requirements for addressing critical-sized segmental bone defects, offering promising avenues for clinical translation. Moreover, the utilization of biodegradable materials ensures compatibility with natural tissue regeneration processes while preventing the need for secondary removal surgeries. Furthermore, each component within the system closely matches the patient's unique anatomy, ensuring an optimal fit and reducing the risk of complications associated with non-customized implants.

The biological properties of the scaffold can be improved through a surface dip coating with natural material, such as collagen, to enhance cell adhesion and growth. Moreover, coating the scaffold with hydroxyapatite may result in improving its overall mechanical properties by supporting the bone formation process, as it contains calcium. Which in turn interacts with stem cells to facilitate their differentiation into osteoblasts and start forming strong bones.

### APPENDIX A: RAW DATA

### 1. Raw Data of the Bone Scaffolds

TABLE 22
Raw Data of microstructural analysis of the scaffold groups.

| Scaffold Pore | | Pore Size | | Standard |
|---|---|---|---|---|
| volume | Trials | [mm] | Mean | Deviation |
| 30% | 1 | 0.533 | 0.530 | 0.016 |
| | 2 | 0.538 | | |
| | 3 | 0.525 | | |
| | 4 | 0.503 | | |
| | 5 | 0.552 | | |
| 40% | 1 | 1.015 | 0.956 | 0.106 |
| | 2 | 1.016 | | |
| | 3 | 0.992 | | |
| | 4 | 0.744 | | |
| | 5 | 1.013 | | |
| 50% | 1 | 1.049 | 1.030 | 0.022 |
| | 2 | 1.029 | | |
| | 3 | 1.015 | | |
| | 4 | 0.998 | | |
| | 5 | 1.060 | | |

TABLE 23
Raw Data of the mechanical properties of the 30% pore volume scaffold.

| | Compressive | | Compressive | Extension | | |
|---|---|---|---|---|---|---|
| | Strength at | | strain at | at | Load | Energy |
| | Maximum | Compressive | Maximum | Maximum | at | at |
| | Load | Modulus | Load | Load | Break | Break |
| Specimen | [MPa] | [MPa] | [mm/mm] | [mm] | [N] | [J] |
| 1 | 46.60519 | 926.5665 | 0.12954 | 1.29771 | 3812.878 | 0.17675 |
| 2 | 44.53454 | 863.523 | 0.1294 | 1.30021 | 3643.475 | 0.1674 |
| 3 | 47.56378 | 927.2706 | 0.12779 | 1.29271 | 3889.499 | 0.18118 |
| 4 | 51.96281 | 925.5054 | 0.13004 | 1.30021 | 4251.197 | 0.20073 |
| 5 | 52.53202 | 915.5102 | 0.12967 | 1.29812 | 4297.765 | 0.18873 |
| 6 | 47.69993 | 861.3064 | 0.12988 | 1.29917 | 3902.441 | 0.16769 |
| Mean | 48.48304 | 903.2804 | 0.129387 | 1.298022 | 3966.209 | 0.180413 |
| Standard | 2.860019 | 29.16549 | 0.000744 | 0.002557 | 234.082 | 0.011741 |
| Deviation | | | | | | |

TABLE 24
Raw Data of the mechanical properties of the 40% pore volume scaffold.

| | Compressive | | Compressive | Extension | | |
|---|---|---|---|---|---|---|
| | Strength at | | strain at | at | Load | Energy |
| | Maximum | Compressive | Maximum | Maximum | at | at |
| | Load | Modulus | Load | Load | Break | Break |
| Specimen | [MPa] | [MPa] | [mm/mm] | [mm] | [N] | [J] |
| 1 | 40.4208 | 816.2369 | 0.12996 | 1.30021 | 3306.92 | 0.1695 |
| 2 | 38.06894 | 722.0519 | 0.13004 | 1.30021 | 3114.507 | 0.13437 |
| 3 | 36.5165 | 833.8281 | 0.13004 | 1.30021 | 2987.5 | 0.14593 |
| 4 | 39.35993 | 836.8454 | 0.13004 | 1.30021 | 3220.127 | 0.15513 |
| 5 | 41.94967 | 748.7975 | 0.13008 | 1.30021 | 3432 | 0.16032 |
| 6 | 44.26152 | 783.8644 | 0.12683 | 1.28417 | 3620.472 | 0.17139 |
| Mean | 40.09623 | 790.2707 | 0.129498 | 1.297537 | 3280.255 | 0.156107 |
| Standard | 2.528395 | 43.11915 | 0.001194 | 0.005978 | 206.6714 | 0.01296 |
| Deviation | | | | | | |

TABLE 25
Raw Data of the mechanical properties of the 50% pore volume scaffold.

| | Compressive | | Compressive | Extension | | |
|---|---|---|---|---|---|---|
| | Strength at | | strain at | at | Load | Energy |
| | Maximum | Compressive | Maximum | Maximum | at | at |
| | Load | Modulus | Load | Load | Break | Break |
| Specimen | [MPa] | [MPa] | [mm/mm] | [mm] | [N] | [J] |
| 1 | 34.57358 | 523.9476 | 0.12975 | 1.29875 | 2828.545 | 0.13417 |
| 2 | 34.68439 | 609.9996 | 0.13004 | 1.30021 | 2837.609 | 0.14305 |
| 3 | 31.4323 | 535.7506 | 0.13004 | 1.30021 | 2571.549 | 0.11999 |
| 4 | 33.81756 | 584.6861 | 0.13004 | 1.30021 | 2766.693 | 0.1299 |
| 5 | 31.53667 | 590.7095 | 0.13004 | 1.30021 | 2580.088 | 0.12736 |
| 6 | 28.36409 | 516.4971 | 0.13004 | 1.30021 | 2320.532 | 0.10566 |
| Mean | 32.40143 | 560.2651 | 0.129992 | 1.299967 | 2650.836 | 0.126688 |
| Standard | 2.233187 | 36.13012 | 0.000108 | 0.000544 | 182.7019 | 0.011701 |
| Deviation | | | | | | |

TABLE 26
Raw Data of the Biodegradation test of the scaffold groups.

| | | Initial | Final | | |
|---|---|---|---|---|---|
| Scaffold Pore | | Weight | Weight | Weight | Average |
| volume | Specimen | [mg] | [mg] | Loss % | % |
| 30% | 1 | 855.7 | 851.9 | 0.444 | 0.544 |
| | 2 | 856.99 | 851.22 | 0.673 | |
| | 3 | 856.32 | 851.92 | 0.514 | |
| 40% | 1 | 760.86 | 756.93 | 0.517 | 0.531 |
| | 2 | 751.03 | 747 | 0.537 | |
| | 3 | 757.28 | 753.19 | 0.540 | |
| 50% | 1 | 644.39 | 641.05 | 0.518 | 0.543 |
| | 2 | 653.64 | 650.07 | 0.546 | |
| | 3 | 644.22 | 640.59 | 0.563 | |

TABLE 27
Raw Data of the cell growth of the scaffold groups.

| Scaffold Pore | | | Average |
|---|---|---|---|
| volume | Specimen | Cell Growth | % |
| 30% | 1 | 0.346 | 0.446667 |
| | 2 | 0.46 | |
| | 3 | 0.534 | |
| 40% | 1 | 0.73 | 0.722667 |
| | 2 | 0.586 | |
| | 3 | 0.852 | |
| 50% | 1 | 0.639 | 0.822 |
| | 2 | 0.869 | |
| | 3 | 0.958 | |

### 2. Raw Data of the Bone Plates

TABLE 28
Raw Data of the mechanical properties of the 5 mm thick plates.

| | | Compressive | |
|---|---|---|---|
| | Compressive | Strength at | Load at |
| | Modulus | Maximum | Break |
| Specimen | [MPa] | Load [MPa] | [N] |
| 1 | 1407.583 | 55.93416 | 2796.708 |
| 2 | 1361.684 | 54.11022 | 2705.511 |
| 3 | 1514.682 | 60.19002 | 3009.501 |
| 4 | 1198.486 | 47.6251 | 2381.255 |
| 5 | 1305.584 | 51.88096 | 2594.048 |
| 6 | 1198.486 | 47.6251 | 2381.255 |
| Mean | 1331.084 | 52.89426 | 2644.713 |
| Standard | 112.7767 | 4.48149 | 224.075 |
| Deviation | | | |

TABLE 29
Raw Data of the mechanical properties of the 6 mm thick plates.

| | | Compressive | |
|---|---|---|---|
| | Compressive | Strength at | Load at |
| | Modulus | Maximum | Break |
| Specimen | [MPa] | Load [MPa] | [N] |
| 1 | 1822.24 | 65.8645 | 3293.225 |
| 2 | 1939.985 | 70.12036 | 3506.018 |
| 3 | 1996.054 | 72.14696 | 3607.348 |
| 4 | 1945.592 | 70.32302 | 3516.151 |
| 5 | 1833.454 | 66.26982 | 3313.491 |
| 6 | 2113.799 | 76.40282 | 3820.141 |
| Mean | 1941.854 | 70.18791 | 3509.396 |
| Standard | 98.80796 | 3.571393 | 178.570 |
| Deviation | | | |

TABLE 30
Raw Data of the mechanical properties of the 7 mm thick plates.

| | | Compressive | |
|---|---|---|---|
| | Compressive | Strength at | Load at |
| | Modulus | Maximum | Break |
| Specimen | [MPa] | Load [MPa] | [N] |
| 1 | 2453.562 | 81.26666 | 4063.333 |
| 2 | 2520.867 | 83.49592 | 4174.796 |
| 3 | 2435.207 | 80.65868 | 4032.934 |
| 4 | 2643.239 | 87.54912 | 4377.456 |
| 5 | 2912.458 | 96.46616 | 4823.308 |
| 6 | 2502.511 | 82.88794 | 4144.397 |
| Mean | 2577.974 | 85.38741 | 4269.371 |
| Standard | 163.7863 | 5.424915 | 271.246 |
| Deviation | | | |

TABLE 31
Raw Data of the Biodegradation test of the plate groups.

| Plate | | Initial Weight | Final Weight | Weight | Average |
|---|---|---|---|---|---|
| Thickness | Specimen | [mg] | [mg] | Loss % | % |
| 5 mm | 1 | 3139.52 | 3126.49 | 0.415 | 0.451 |
| | 2 | 3002.86 | 2989.01 | 0.46123 | |
| | 3 | 3084.96 | 3070.25 | 0.4768 | |
| 6 mm | 1 | 3751.97 | 3734.52 | 0.465 | 0.472 |
| | 2 | 3781.18 | 3762.63 | 0.491 | |
| | 3 | 3892.29 | 3874.43 | 0.459 | |
| 7 mm | 1 | 4693.23 | 4672.44 | 0.443 | 0.432 |
| | 2 | 4714.72 | 4694.14 | 0.437 | |
| | 3 | 4710.36 | 4690.79 | 0.415 | |

### 3. Raw Data of the Bone Screws

TABLE 32
Raw Data of the mechanical properties
of the 6 mm diameter screws.

| | | | Peak |
|---|---|---|---|
| | Threshold | Peak Failure | Clamping |
| | Torque | Torque | Torque |
| Specimen | [N · mm] | [N · mm] | [N · mm] |
| 1 | 346.6726 | 322.8564 | 334.765 |
| 2 | 361.7675 | 348.0094 | 354.888 |
| 3 | 386.3365 | 387.9254 | 387.131 |
| 4 | 377.5446 | 328.9032 | 353.224 |
| 5 | 351.953 | 332.2945 | 342.124 |
| 6 | 387.46 | 313.5536 | 350.507 |
| Mean | 368.6224 | 338.9238 | 353.773 |
| Standard | 16.09815 | 24.25515 | 16.442 |
| Deviation | | | |

TABLE 33
Raw Data of the mechanical properties
of the 7 mm diameter screws.

| | | | Peak |
|---|---|---|---|
| | Threshold | Peak Failure | Clamping |
| | Torque | Torque | Torque |
| Specimen | [N · mm] | [N · mm] | [N · mm] |
| 1 | 374.9736 | 338.0924 | 356.533 |
| 2 | 355.6687 | 398.2454 | 376.957 |
| 3 | 387.0297 | 340.2851 | 363.658 |
| 4 | 376.5632 | 389.385 | 382.974 |
| 5 | 321.7122 | 322.9476 | 322.330 |
| 6 | 394.8839 | 369.0443 | 381.964 |
| Mean | 368.4719 | 359.6666 | 364.0693 |
| Standard | 24.1527 | 27.8353 | 20.988 |
| Deviation | | | |

TABLE 34
Raw Data of the mechanical properties
of the 8 mm diameter screws.

| | | | Peak |
|---|---|---|---|
| | Threshold | Peak Failure | Clamping |
| | Torque | Torque | Torque |
| Specimen | [N · mm] | [N · mm] | [N · mm] |
| 1 | 387.8562 | 592.2696 | 490.063 |
| 2 | 357.7693 | 601.3874 | 479.578 |
| 3 | 390.2845 | 593.5871 | 491.936 |
| 4 | 375.6593 | 499.3852 | 437.522 |
| 5 | 389.0039 | 558.2856 | 473.645 |
| 6 | 363.6942 | 578.8987 | 471.297 |
| Mean | 377.3779 | 570.6356 | 474.007 |
| Standard | 12.82077 | 34.73206 | 18.023 |
| Deviation | | | |

TABLE 35
Raw Data of the Biodegradation test of the screw groups.

| Screw | | Initial Weight | Final Weight | Weight | Average |
|---|---|---|---|---|---|
| Diameter | Specimen | [mg] | [mg] | Loss % | % |
| 6 mm | 1 | 274.28 | 273.24 | 0.379 | 0.363 |
| | 2 | 278.31 | 277.39 | 0.331 | |
| | 3 | 269.31 | 268.29 | 0.379 | |
| 7 mm | 1 | 326.6 | 324.82 | 0.545 | 0.532 |
| | 2 | 326.25 | 324.49 | 0.539 | |
| | 3 | 329.59 | 327.9 | 0.5128 | |
| 8 mm | 1 | 474.56 | 472.54 | 0.426 | 0.447 |
| | 2 | 477.18 | 474.99 | 0.459 | |
| | 3 | 474.85 | 472.68 | 0.457 | |

The above-described hardware description is a non-limiting example of corresponding structure for performing the functionality described herein.

Numerous modifications and variations of the present disclosure are possible in light of the above teachings. It is therefore to be understood that the invention may be practiced otherwise than as specifically described herein.

1. A method of repairing a bone fracture in a patient, the method comprising:
imaging the bone fracture in need of repair;
generating a fracture model of the bone fracture based on the imaging;
forming a model of a bone repair implant based on the fracture model;
manufacturing the bone repair implant using an additive manufacturing method based on the model; and
surgically implanting the bone repair implant adjacent to the bone fracture in the patient,
wherein
the bone repair implant comprises a bone scaffold, a support plate, and a screw;
the bone scaffold comprises a network of scaffold members defining an interconnected pore network and has a shape configured to replace a missing bone portion from the bone fracture;
the support plate has an inner surface profile configured to match an outer surface profile of the bone fracture in need of repair in an area covered by the support plate and an outer surface profile which smoothly transitions to match an outer surface profile of the bone fracture in need of repair in an area not covered by the support plate such that the bone repair implant is devoid of sharp edges;
the bone repair implant is constructed of a biodegradable material which is at least one selected from the group consisting of polylactic acid, polyglycolic acid, and poly lactic acid glycolic acid.

2. The method of claim 1, wherein
the bone fracture in need of repair includes a proximal bone portion, the missing bone portion, and a distal bone portion.

3. The method of claim 2, wherein
the support plate is configured to secure the bone scaffold, a proximal bone portion, and a distal bone portion in a repair configuration.

4. The method of claim 3, wherein
the support plate is configured to be attached to at least one selected from the group consisting of the proximal bone portion and the distal bone portion using the screw.

5. The method of claim 3, wherein
the support plate is configured to be attached to both the proximal bone portion using a first screw and the distal bone portion using a second screw.

6. The method of claim 4, wherein
the support plate is configured to be further attached to the bone scaffold using a screw.

7. The method of claim 1, wherein
the biodegradable material comprises polylactic acid.

8. The method of claim 1, wherein
the bone scaffold has a pore volume of 25 to 60% of a total volume of the bone scaffold.

9. The method of claim 1, wherein
the support plate has a thickness of 5 to 7 mm.

10. The method of claim 1, wherein
the screw has a shank diameter of at least 8 mm.

11. The method of claim 1, wherein
the screw has a head diameter of at least 9 mm.

12. The method of claim 1, further comprising
calculating a mechanical stress response of the model of a bone repair implant to an expected mechanical stress.

13. The method of claim 12, wherein
the expected mechanical stress is based on a location of the bone fracture in need of repair.

14. The method of claim 12, wherein
the expected mechanical stress comprises at least one selected from the group consisting of a compressive stress, a torsional stress, and a bending stress.

15. The method of claim 12, further comprising
when the mechanical stress response of the model of the bone repair implant to the expected mechanical stress is calculated to be unacceptable, adjusting the model of the bone repair implant to produce an acceptable mechanical stress response.

16. The method of claim 15, wherein
the bone repair implant comprises a bone scaffold, a support plate, and a screw; and
the adjusting of the model of the bone repair implant includes at least one selected from the group consisting of adjusting a pore volume of the bone scaffold, adjusting a thickness of the support plate, adjusting a length of the support plate, adjusting width profile of the support plate, adjusting a screw placement location, and adjusting a screw number of the bone repair implant.

17. The method of claim 1, wherein the imaging is at least one selected from the group consisting of computerized tomography, magnetic resonance imaging, and nuclear bone scanning.

18. The method of claim 5, wherein the bone repair implant further comprises a radiopaque marker.

19. The method of claim 1, wherein the additive manufacturing method is at least one selected from the group consisting of fused deposition modeling, selective laser sintering, and stereolithography.

20. The method of claim 1, wherein
the patient does not require an additional surgery to remove the bone repair implant.
